(19) United States
(12) Patent Application Publication
(10) Pub. No.: US 2017/0362508 A1
(43) Pub. Date: Dec. 21, 2017

(54) POLYMERIZABLE COMPOUND AND OPTICALLY ANISOTROPIC BODY

(71) Applicant: DIC Corporation, Tokyo (JP)

(72) Inventors: Masahiro Horiguchi, Kita-adachi-gun (JP); Akihiro Koiso, Kita-adachi-gun (JP)

(73) Assignee: DIC Corporation, Tokyo (JP)

(21) Appl. No.: 15/542,537

(22) PCT Filed: Jan. 14, 2016
(86) PCT No.: PCT/JP2016/050983

§ 371 Date: Jul. 10, 2017

(30) Foreign Application Priority Data
Jan. 16, 2015 (JP) 2015-006294

(51) Int. Cl. C09K 19/34; C08F 20/38; C09K 19/38; C09K 19/32; C07C 323/12; C07D 285/135; C07D 417/12; C07D 417/06; C07D 409/12; C07D 409/04; C07D 305/06; G02B 5/30; C08F 20/36

(52) U.S. Cl. CPC C09K 19/3497; G02B 5/3016; C08F 20/38; C09K 19/3823; C09K 19/3809; C09K 19/3491; C09K 19/3402; C09K 19/322; C07C 323/12; C08F 20/36; C07D 417/12; C07D 417/06; C07D 409/12; C07D 409/04; C07D 305/06; C07D 285/135; C09K 2019/3416

(57) ABSTRACT

A problem of the present invention is to provide a polymerizable compound and a polymerizable composition which cause little decrease in retardation and discoloration when a film-shaped polymer produced by polymerization is irradiated with ultraviolet/visible light for a long time at high temperature. A further problem is to provide a polymer produced by polymerizing the polymerizable composition and an optically anisotropic body using the polymer. As a result, a compound useful as a component of a polymerizable composition was obtained. An optically anisotropic body using a polymerizable liquid crystal composition containing the compound of the present invention is useful for application to an optical film and the like.

### TECHNICAL FIELD

The present invention relates to a compound having a polymerizable group, a polymerizable composition containing the compound, a polymerizable liquid crystal composition, and an optically anisotropic body using the polymerizable liquid crystal composition.

### BACKGROUND ART

Compounds (polymerizable compounds) having polymerizable groups are used as various optical materials. For example, a polymer having uniform alignment can be produced by aligning, in a liquid crystal state, a polymerizable composition containing a polymerizable compound, and then polymerizing the composition. Such a polymer can be used for a polarization plate, a retardation plate, and the like necessary for displays. In many cases, a polymerizable composition containing two or more types of polymerizable compounds is used for satisfying the required optical characteristics, polymerization rate, solubility, melting point, glass transition temperature, polymer transparency, mechanical strength, surface hardness, heat resistance, and light resistance. In this case, the polymerizable compounds used are required to impart good physical properties to the polymerizable composition without adversely affecting other characteristics.

Liquid crystal displays used outdoors or in a place exposed to a high temperature are required to have higher-reliability than usual liquid crystal displays. For example, in the field of mobile products, in-vehicle products, and the like, particularly high heat resistance and light resistance are required. Various polymerizable compounds have been reported for applications to retardation films for improving viewing angles of liquid crystal displays. However, when irradiated with ultraviolet/visible light for a long time at high temperature, the films produced by using the polymerizable compounds may be decreased in retardation or may be discolored (Patent Literature 1 and 2). When the films which may be decreased in retardation or may be discolored are used in, for example, liquid crystal displays of a mobile product, an in-vehicle product, and the like, brightness unevenness in screens or unnatural colors occur due to long-term use, thereby causing the problem of significantly decreasing the quality of the products. Therefore, there has been demand for development of a polymerizable compound capable of solving the problem.

### CITATION LIST

### Patent Literature

PTL 1: Japanese Unexamined Patent Application Publication (Translation of PCT Application) No. 2010-537955

PTL 2: Japanese Unexamined Patent Application Publication No. 2008-291218

### SUMMARY OF INVENTION

### Technical Problem

A problem to be solved by the present invention is to provide a polymerizable compound and a polymerizable composition which cause little decrease in retardation and discoloration when a film-shaped polymer produced by polymerization is irradiated with ultraviolet/visible light for a long time at high temperature. A further problems is to provide a polymer produced by polymerizing the polymerizable composition and an optically anisotropic body using the polymer.

### Solution to Problem

As a result of earnest investigation for solving the problems, the inventors led to the development of a compound described below. That is, the present invention provides a polymerizable liquid crystal compound having an absorption maximum wavelength λomax at 320 nm to 420 nm in an in-plane direction perpendicular to an alignment direction when aligned on a horizontally-aligned substrate, and also provides a polymerizable composition containing the compound, a polymerizable liquid crystal composition, a polymer produced by polymerizing the polymerizable liquid crystal composition, and an optically anisotropic body using the polymer.

### Advantageous Effects of Invention

A compound of the present invention causes little decrease in adhesion and little discoloration when a film-shaped polymer produced by adding to a polymerizable composition and polymerizing the composition is irradiated with ultraviolet/visible light for a long time at high temperature, and thus the compound is useful as a component of a polymerizable composition. Also, an optically anisotropic body using a polymerizable liquid crystal composition containing the compound of the present invention is useful for applications to optical materials such as a retardation film and the like.

### DESCRIPTION OF EMBODIMENTS

The best mode of a polymerizable liquid crystal compound according to the present invention is described below and, in the present invention, the “liquid crystalline compound” is intended to represent a compound having a mesogenic skeleton and the compound by itself may not have liquid crystallinity.

The present invention provides a polymerizable liquid crystal compound in which when aligned on a horizontally-aligned substrate, an absorption maximum wavelength λomax in an in-plane direction perpendicular to an alignment direction lies at 320 nm to 420 nm, and also provides a polymerizable composition containing the compound, a polymerizable liquid crystal composition, a polymer produced by polymerizing the polymerizable liquid crystal composition, and an optically anisotropic body using the polymer.

The absorption maximum wavelength λomax in an in-plane direction perpendicular to an alignment direction can be measured as follows. Measurement is performed by using a spectrophotometer, and an absorption spectrum is measured by disposing a film to be evaluated and a polarization plate on the detector-side surface of the film so that the alignment direction of the film is perpendicular to the polarization direction of the polarization plate (refer to a drawing). A compound to be evaluated may be applied alone on a substrate, may be diluted with a solvent and applied, or may be mixed with another component and applied. When a plurality of absorption maximums are present in 320 nm to 420 nm, the wavelength showing the maximum value among the plurality of absorption maximums is defined as λomax.

From the viewpoint that the resultant film causes little decrease in retardation and little discoloration, when the film is aligned on a horizontally-aligned substrate, at the wavelength λomax, absorbance Ae in the direction parallel to the alignment direction and absorbance Ao in the in-plane direction perpendicular to the alignment direction preferably satisfy a formula (Formula I) below.

Ao/Ae>1 (Formula I)

The absorbance Ao at the wavelength λomax in the in-plane direction perpendicular to the alignment direction is determined by the method for measuring λomax described above. The absorbance Ae in the direction parallel to the alignment direction can be determined by measuring an absorption spectrum by disposing a film to be evaluated and a polarization plate on the detector-side surface of the film so that the alignment direction of the film is parallel to the polarization direction of the polarization plate (refer to the drawing).

Also, from the viewpoint that the resultant film causes little decrease in retardation and little discoloration, it is more preferred that the formula (Formula I) is satisfied and λomax is 330 nm to 370 nm.

Even more preferred is a compound represented by general formula (I) below,

[Chem. 1]

P¹S¹—X¹ₖA¹¹-Z¹¹ₘ₁M¹Z¹²-A¹²ₘ₂R¹ (I)

(in the formula, P¹ represents a polymerizable group; S¹ represents a spacer group or a single bond, and when a plurality of S¹ are present, they may be the same or different; X¹ represents —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —N═N—, —CH═N—N═CH—, —CF═CF—, —C≡C—, or a single bond, and when a plurality of X¹ are present, they may be the same or different (wherein P—(S—X)ₖ— does not contain an —O—O— bond); A¹¹ and A¹² each independently represent a 1,4-phenylene group, a 1,4-cyclohexylene group, a pyridine-2,5-diyl group, a pyrimidine-2,5-diyl group, a naphthalene-2,6-diyl group, a naphthalene-1,4-diyl group, a tetrahydronaphthalene-2,6-diyl group, a decahydronaphthalene-2,6-diyl group, or a 1,3-dioxane-2,5-diyl group, which may be unsubstituted or substituted by one or more L, and when a plurality of A¹¹ and/or A¹² are present, they may be the same or different; Z¹¹ and Z¹² each independently represent —O—, —S—, —OCH₂—, —CH₂O—, —CH₂CH₂—, —CO—, —COO—, —OCO—, —CO—S—, —S—CO—, —O—CO—O—, —CO—NH—, —NH—CO—, —OCO—NH—, —NH—COO—, —NH—CO—NH—, —NH—O—, —O—NH—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —N═N—, —CH═N—, —N═CH—, —CH═N—N═CH—, —CF═CF—, —C≡C—, or a single bond, and when a plurality of Z¹¹ and/or Z¹² are present are present, they may be the same or different; R¹ represents a hydrogen atom, a fluorine atom, a chlorine atom, a bromine atom, an iodine atom, a pentafluorosulfuranyl group, a cyano group, a nitro group, an isocyano group, a thioisocyano group, or a linear or branched alkyl group having 1 to 20 carbon atoms in which one —CH₂— or two or more unadjacent —CH₂— may be each independently substituted by —O—, —S—, —CO—, —COO—, —OCO—, —CO—S—, —S—CO—, —O—CO—O—, —CO—NH—, —NH—CO—, or —C≡C—, and any hydrogen atom in the alkyl group may be substituted by a fluorine atom, or R¹ may represent a group represented by —(X^{R}—S^{R})_{kR}—P^{R} (wherein P^{R} represents a polymerizable group; S^{R} represents a spacer group or a single bond, and when a plurality of S^{R} are present, they may be the same or different; X^{R} represents —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —N═N—, —CH═N—N═CH—, —CF═CF—, —C≡C—, or a single bond, and when a plurality of X^{R} are present, they may be the same or different (wherein —(X^{R}—S^{R})_{kR}—P^{R} does not contain an —O—O— bond); and kR represents an integer of 0 to 8); M¹ represents a divalent hydrocarbon group containing a conjugated system; L represents a fluorine atom, a chlorine atom, a bromine atom, an iodine atom, a pentafluorosulfuranyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a linear or branched alkyl group having 1 to 20 carbon atoms in which one —CH₂— or two or more unadjacent —CH₂— may be each independently substituted by —O—, —S—, —CO—, —COO—, —OCO—, —CO —S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, or —C≡C—, and when a plurality of L are present, they may be the same or different, and any hydrogen atom in the alkyl group may be substituted by a fluorine atom, or L may represent a group represented by —(X^{L}—S^{L})_{kL}—P^{L} (wherein P^{L} represents a polymerizable group; S^{L} represents a spacer group or a single bond, and when a plurality of S^{L} are present, they may be the same or different; X^{L} represents —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —N═N—, —CH═N—N═CH—, —CF═CF—, —C≡C—, or a single bond, and when a plurality of X^{L} are present, they may be the same or different (wherein —(X^{L}—S^{L})_{kL}—P^{L} does not contain an —O—O— bond); and kL represents an integer of 0 to 8); k represents an integer of 0 to 8; m1 and m2 each independently represent an integer of 0 to 5, and m1+m2 represents an integer of 1 to 5).

In the general formula (I), P¹ represents a polymerizable group and preferably represents a group selected from formula (P-1) to formula (P-20) below.

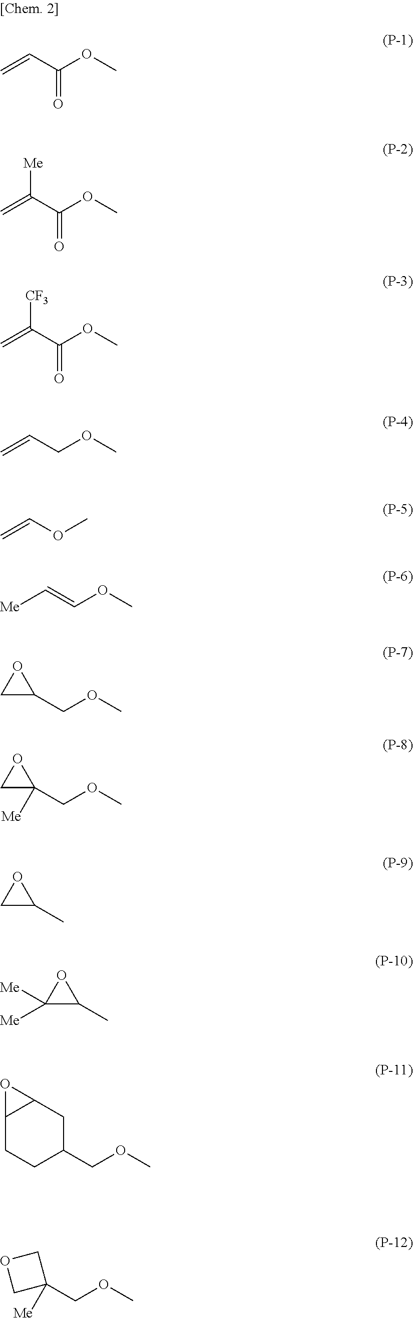

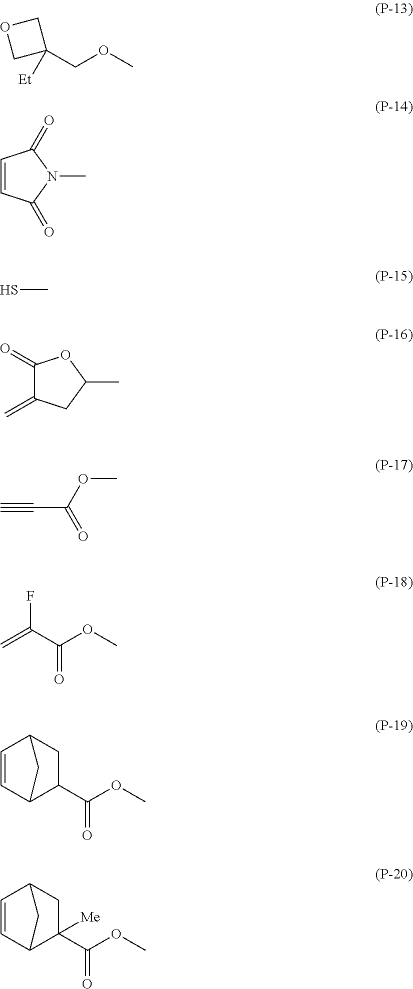

These polymerizable groups are polymerized by radical polymerization, radical addition polymerization, cationic polymerization, and anionic polymerization. In particular, when ultraviolet polymerization is performed as a polymerization method, the formula (P-1), the formula (P-2), the formula (P-3), the formula (P-4), the formula (P-5), the formula (P-7), the formula (P-11), the formula (P-13), the formula (P-15) or the formula (P-18) is preferred, the formula (P-1), the formula (P-2), the formula (P-7), the formula (P-11), or the formula (P-13) is more preferred, the formula (P-1), the formula (P-2), or the formula (P-3) is even more preferred, and the formula (P-1) or the formula (P-2) is particularly preferred.

S¹ represents a spacer group or a single bond, and when a plurality of S¹ are present, they may be the same or different. Also, S¹ preferably represents as the spacer group an alkylene group having 1 to 20 carbon atoms in which one —CH₂— or two or more unadjacent —CH₂— may be each independently substituted by —O—, —COO—, —OCO—, —OCO—O—, —CO—NH—, —NH—CO—, —CH═CH—, or —C≡C—. When a plurality of S¹ are present from the viewpoint of easy availability of raw materials and easy synthesis, they may be the same or different and more preferably each independently represent an alkylene group having 1 to 10 carbon atoms, in which one —CH₂— or two or more unadjacent —CH₂— may be each independently substituted by —O—, —COO—, or —OCO—, or a single bond, and even more preferably represent an alkylene group having 1 to 10 carbon atoms or a single bond. When a plurality of S¹ are present, they may be the same or different and particularly preferably each independently represent an alkylene group having 1 to 8 carbon atoms.

From the viewpoint of easy availability of raw materials and easy synthesis, when a plurality of X¹ are present, they may be the same or different and preferably each independently represent —O—, —S—, —OCH₂—, —CH₂O—, —COO—, —OCO—, —CO—S—, —S—CO—, —O—CO—O—, —CO—NH—, —NH—CO—, —COO—CH₂CH₂—, —OCO—CH₂CH₂—, —CH₂CH₂—COO—, —CH₂CH₂—OCO—, or a single bond, and even more preferably represent —O—, —OCH₂—, —CH₂O—, —COO—, —OCO—, —COO—CH₂CH₂—, —OCO—CH₂CH₂—, —CH₂CH₂—COO—, —CH₂CH₂—OCO—, or a single bond. When a plurality of X¹ are present, they may be the same or different and particularly preferably each independently represent —O—, —COO—, —OCO—, or a single bond.

In addition, k represents an integer of 0 to 8, and from the viewpoint of easy availability of raw materials and easy synthesis, k preferably represents an integer of 0 to 4, more preferably represents an integer of 0 to 3, even more preferably represents an integer of 0 to 2, and particularly preferably represents 1.

From the viewpoint of easy availability of raw materials and easy synthesis, A¹¹ and A¹² preferably each independently represent a 1,4-phenylene group, a 1,4-cyclohexylene group, or a naphthalene-2,6-diyl group, which may be unsubstituted or substituted by one or more L, more preferably each independently represent a group selected from formula (A-1) to formula (A-11) below,

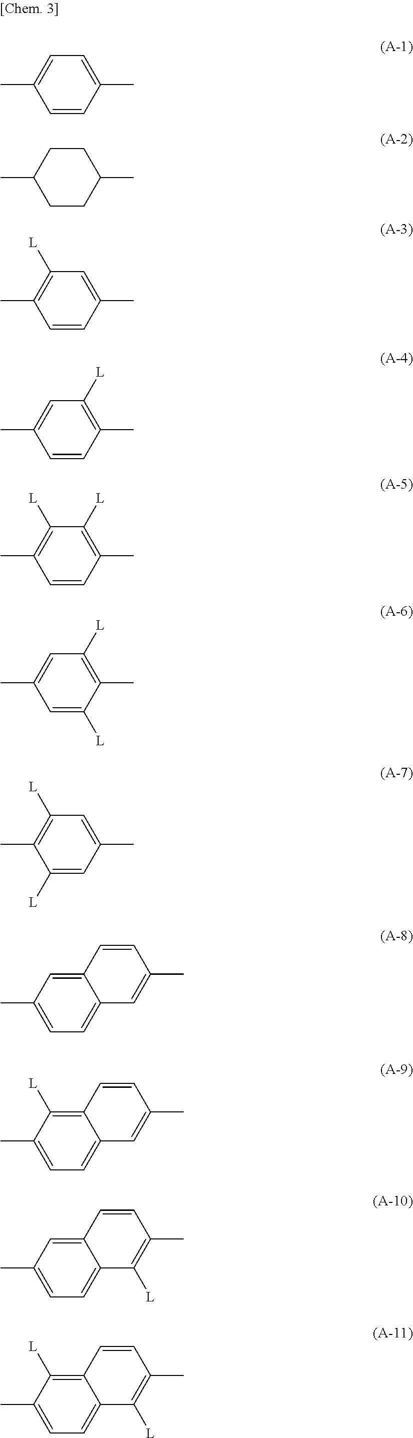

even more preferably each independently represent a group selected from the formula (A-1) to the formula (A-8), and particularly preferably each independently represent a group selected from the formula (A-1) to the formula (A-4).

From the viewpoint of liquid crystallinity of the compound, easy availability of raw materials, and easy synthesis, Z¹¹ and Z¹² preferably each independently represent a single bond, —OCH₂—, —CH₂O—, —COO—, —OCO—, —CF₂O—, —OCF₂—, —CH₂CH₂—, —CF₂CF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —CH═CH—, —CF═CF—, —C≡C—, or a single bond, more preferably each independently represent —OCH₂—, —CH₂O—, —CH₂CH₂—, —COO—, —OCO—, —COO—CH₂CH₂—, —OCO—CH₂CH₂—, —CH₂CH₂—COO—, —CH₂CH₂—OCO—, —CH═CH—, —C≡C—, or a single bond, even more preferably each independently represent —OCH₂—, —CH₂O—, —CH₂CH₂—, —COO—, —OCO—, —COO—CH₂CH₂—, —OCO—CH₂CH₂—, —CH₂CH₂—COO—, —CH₂CH₂—OCO—, or a single bond, and particularly preferably each independently represent —OCH₂—, —CH₂O—, —COO—, —OCO—, or a single bond.

From the viewpoint of liquid crystallinity and easy synthesis, R¹ preferably represents a hydrogen atom, a fluorine atom, a chlorine atom, a cyano group, a linear or branched alkyl group having 1 to 12 carbon atoms in which one —CH₂— or two or more unadjacent —CH₂— may be each independently substituted by —O—, —COO—, —OCO—, or —O—CO—O—, or a group represented by —(X^{R}—S^{R})_{kR}—P^{R}, more preferably represents a hydrogen atom, a fluorine atom, a chlorine atom, a cyano group, a linear alkyl group or linear alkoxy group having 1 to 12 carbon atoms, or a group represented by —(X^{R}—S^{R})_{kR}—P^{R}, and particularly preferably represents a group represented by —(X^{R}—S^{R})_{kR}—P^{R}. P^{R} represents a polymerizable group; S^{R} represents a spacer group or a single bond, and when a plurality of S^{R} are present, they may be the same or different; X^{R} represents —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —N═N—, —CH═N—N═CH—, —CF═CF—, —C≡C—, or a single bond, and when a plurality of X are present, they may be the same or different (wherein —(X^{R}—S^{R})_{kR}—P^{R} does not contain an —O—O— bond); and kR represents an integer of 0 to 8. Preferred structures of P^{R}, S^{R}, X^{R}, and kR are the same as the preferred structures of P¹, S¹, X¹, and k, respectively.

M¹ represents a divalent hydrocarbon group containing a conjugated system, and from the viewpoint of difficulty in occurrence of retardation decrease and discoloration, the total number of π electrons contained in M¹ is preferably 4to 50 and more preferably 4 to 24. From the viewpoint of liquid crystallinity, easy availability of raw materials, and easy synthesis, M¹ preferably represents a group represented by formula (I-M) below,

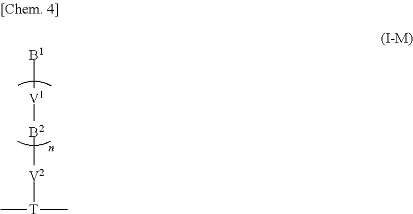

(in the formula, T represents a trivalent hydrocarbon group; B¹ represents a hydrogen atom, a methyl group, a methylidene group, or a cyclic hydrocarbon group, which may be unsubstituted or substituted by one or more L^{B}; B² represents a single bond, a double bond, or a divalent cyclic hydrocarbon group, which may be unsubstituted or substituted by one or more L^{B}; L^{B} represents a fluorine atom, a chlorine atom, a bromine atom, an iodine atom, a pentafluorosulfuranyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a linear or branched alkyl group having 1 to 20 carbon atoms in which one —CH₂— or two or more unadjacent —CH₂— may be each independently substituted by —O—, —S—, —CO—, —COO—, —OCO—, —CO—S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, or —C≡C—, any hydrogen atom in the alkyl group may be substituted by a fluorine atom, and when a plurality of L^{B} are present, they may be the same or different; V¹ and V² each represent a single bond, a double bond, or a divalent bonding group; n represents an integer of 0 to 10; and bonding groups which connect B¹—V¹, V¹—B², B²—V¹, B²—V², and V²-T may be each a single bond or a double bond).

From the viewpoint of liquid crystallinity, easy availability of raw materials, and easy synthesis, T preferably represents a group selected from formula (T-1) to formula (T-22) below,

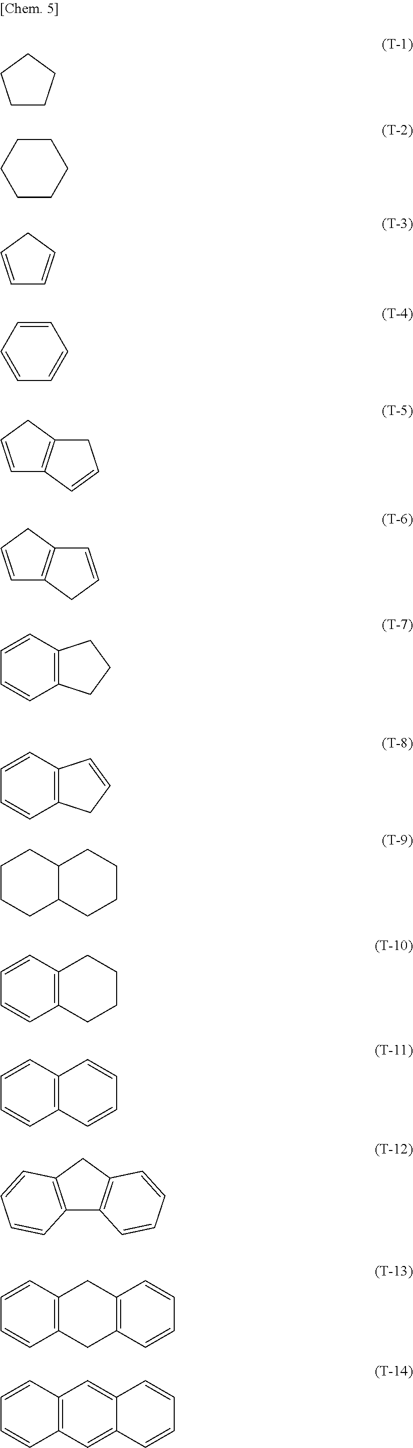

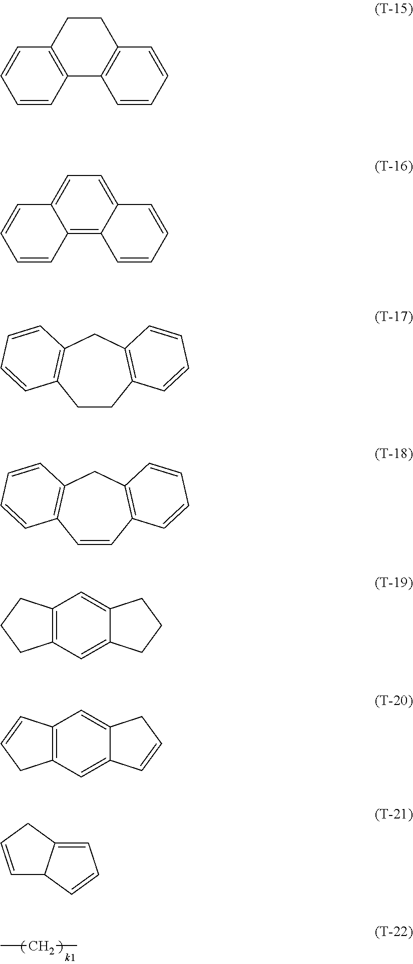

(in the formulae, a bond may be present at any desired position, any desired —CH═ may be each independently replaced by —N═, —CH₂— may be each independently substituted by —O—, —S—, —NR⁰— (wherein R⁰ represents a hydrogen atom or an alkyl group having 1 to 20 carbon atoms), —CS—, or —CO— without containing a —O—O— bond; the expression “a bond may be present at any desired position” represents that, for example, when T is a trivalent group, three bonds are present at any desired positions (this is true for the later expression “a bond may be present at any desired position” in the present invention), and these groups may be unsubstituted or substituted by one or more L^{T}; L^{T} represents a fluorine atom, a chlorine atom, a bromine atom, an iodine atom, a pentafluorosulfuranyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a linear or branched alkyl group having 1 to 20 carbon atoms in which one —CH₂— or two or more unadjacent —CH₂— may be each independently substituted by —O—, —S—, —CO—, —COO—, —OCO—, —CO—S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, or —C≡C—, any hydrogen atom in the alkyl group may be substituted by a fluorine atom, and when a plurality of L^{T} are present, they may be the same or different; and k1 represents an integer of 1 to 20 carbon atoms). T more preferably represents a group selected from the formula (T-4), the formula (T-7), the formula (T-8), and the formula (T-11), and even more preferably represents a group selected from the formula (T-4) and the formula (T-11).

When T represents a group selected from the formula (T-4), more specifically, T preferably represents a group represented by formula (T-4-1) or formula (T-4-2) below,

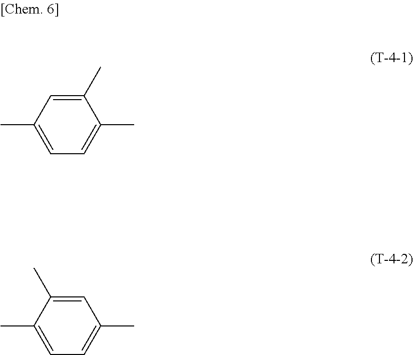

when T represents a group selected from the formula (T-7), more specifically, T preferably represents a group selected from formula (T-7-1) to formula (T-7-21) below,

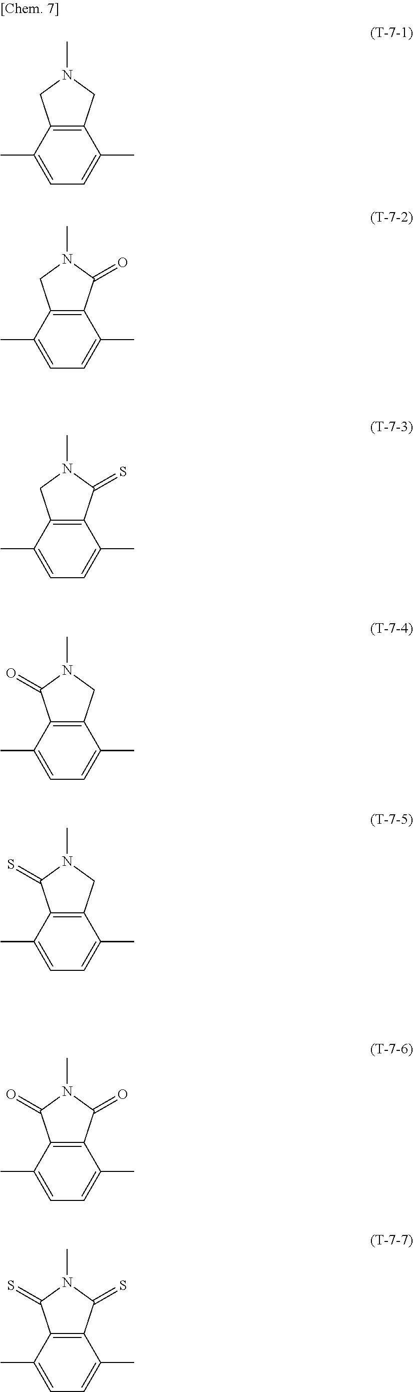

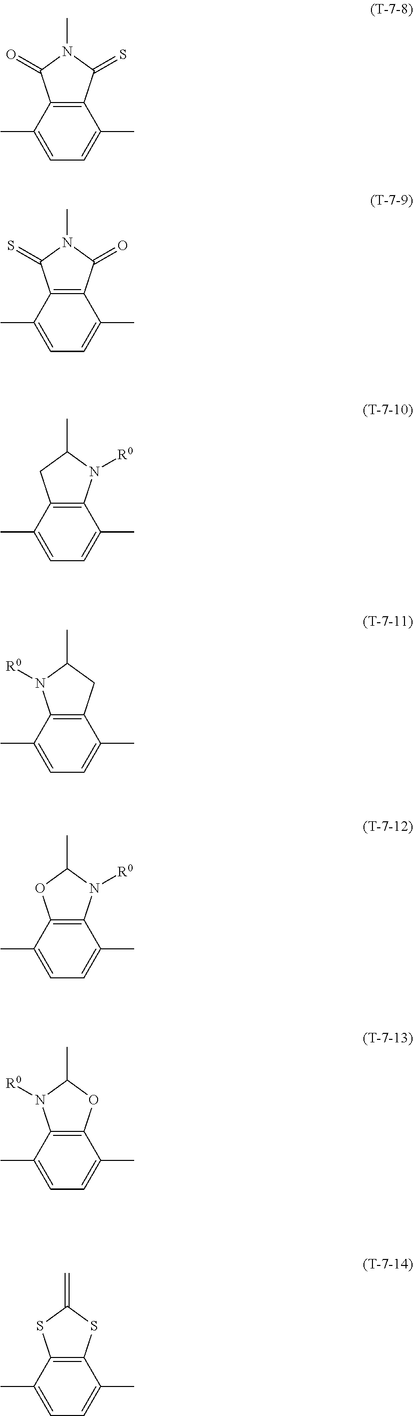

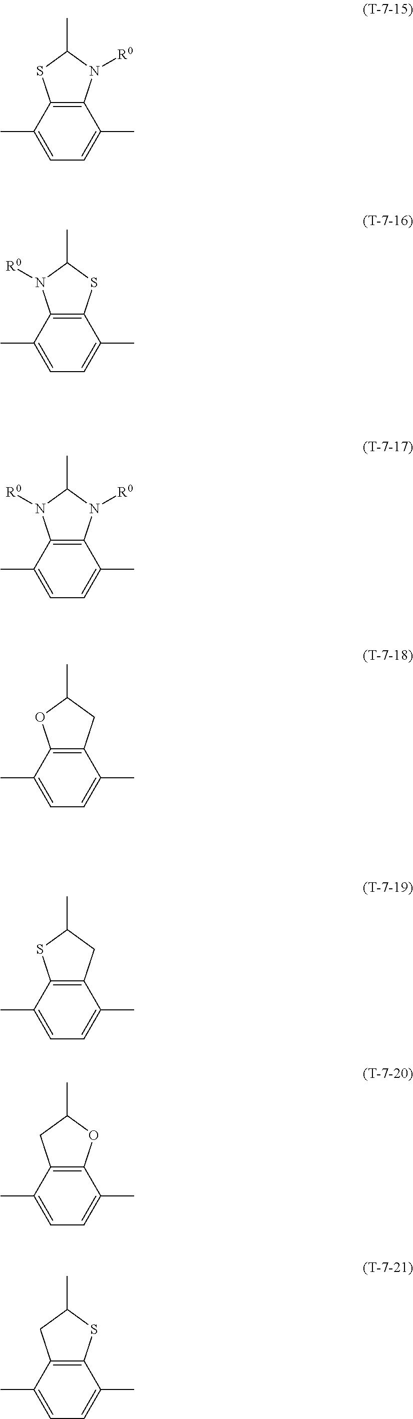

(in the formulae, R⁰ represents a hydrogen atom or an alkyl group having 1 to 20 carbon atoms); when T represents a group selected from the formula (T-8), more specifically, T preferably represents a group selected from formula (T-8-1) to formula (T-8-16) below,

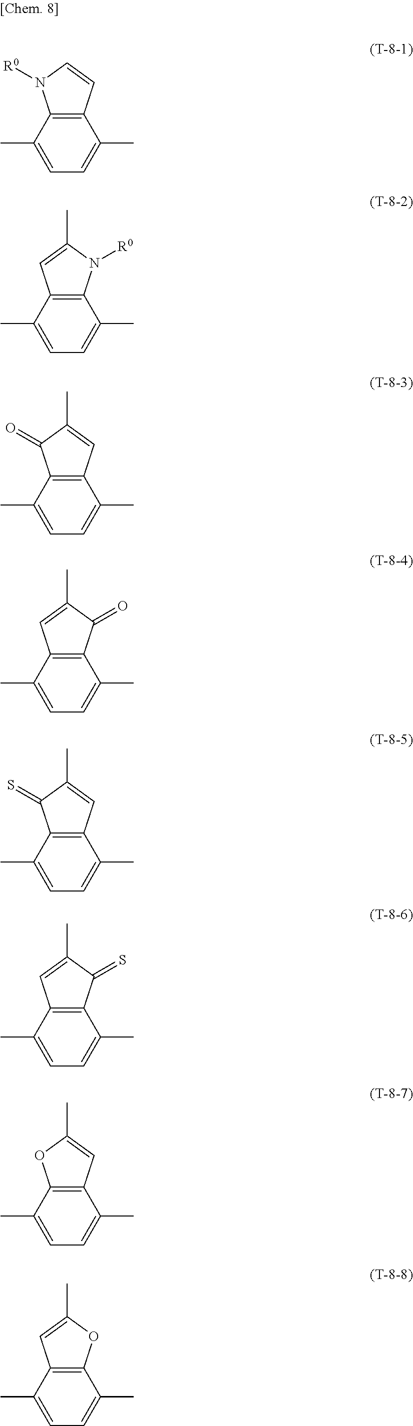

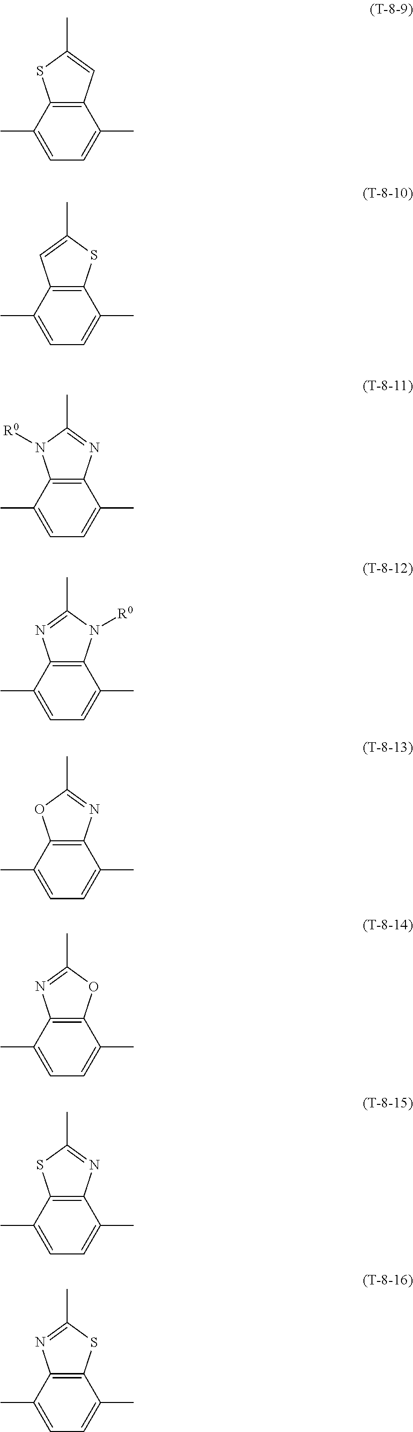

(in the formulae, R⁰ represents a hydrogen atom or an alkyl group having 1 to 20 carbon atoms); and when T represents a group selected from the formula (T-11), more specifically, T preferably represents a group selected from formula (T-11-1) to formula (T-11-4) below,

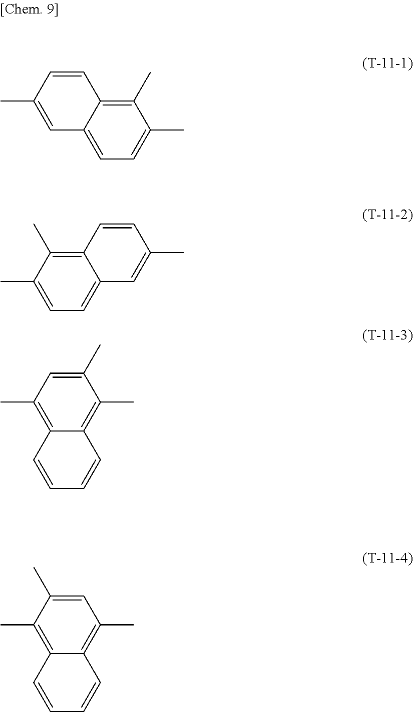

B¹ represents a hydrogen atom or a methyl group, a methylidene group, or a cyclic hydrocarbon group, which may be unsubstituted or substituted by one or more L^{B}; and from the viewpoint of liquid crystallinity, easy availability of raw materials, and easy synthesis, B¹ preferably represents a hydrogen atom or a methyl group or a methylidene group, which may be unsubstituted or substituted by one or more L^{B}, or a group selected from formula (B-1-1) to formula (B-1-21) bellow,

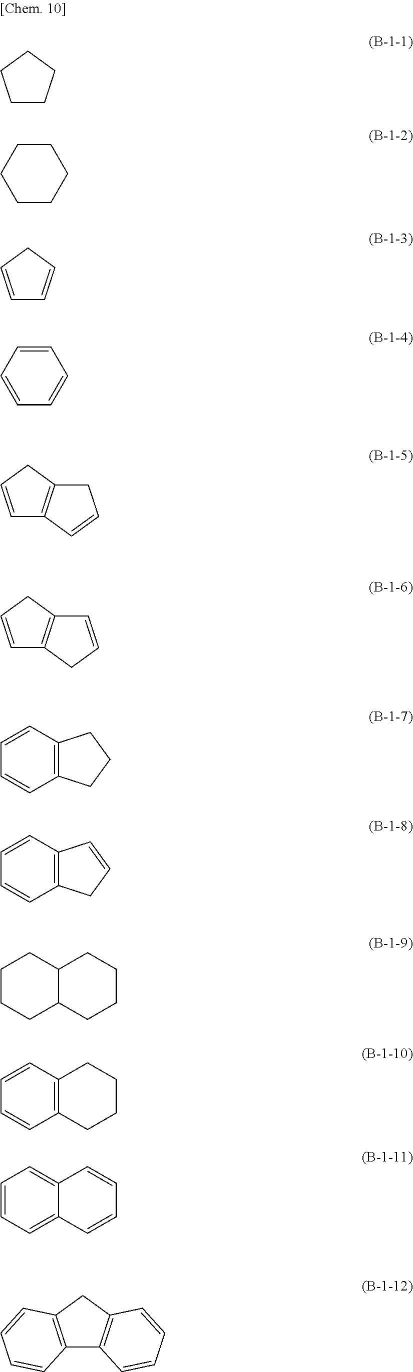

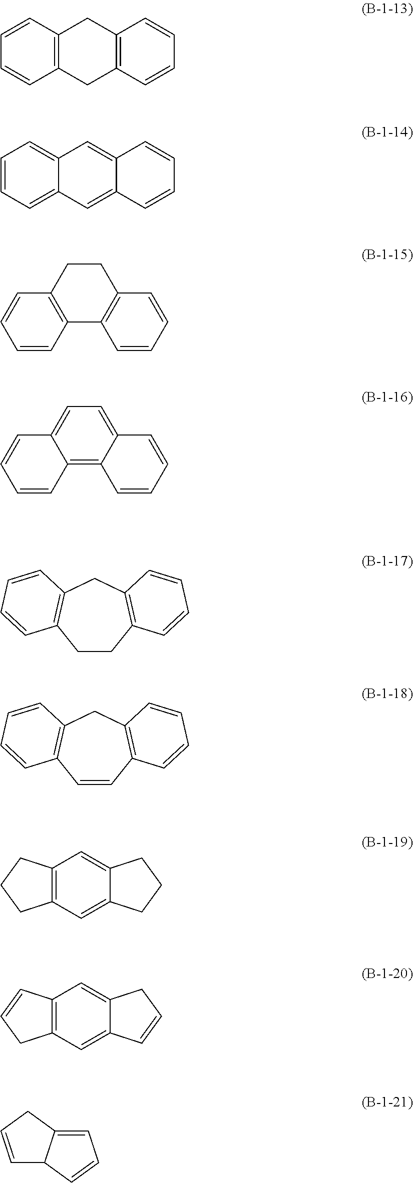

(in the formulae, a ring structure may have a bond at any desired position, any desired —CH═ may be each independently replaced by —N═, —CH₂— may be each independently substituted by —O—, —S—, —NR⁰— (wherein R⁰ represents a hydrogen atom or an alkyl group having 1 to 20 carbon atoms), —CS—, or —CO— without containing a —O—O— bond, and these groups may be unsubstituted or substituted by one or more substituents L^{B}; B¹ more preferably represents a hydrogen atom, a methyl group which may be unsubstituted or substituted by one or more L^{B}, a methylidene group which may be unsubstituted or substituted by one or more L^{B}, or a group which may be unsubstituted or substituted by one or more L^{B} and are selected from the formula (B-1-3), the formula (B-1-4), the formula (B-1-8), the formula (B-1-10), and the formula (B-1-11); and B¹ even more preferably represents a hydrogen atom, a methyl group which may be unsubstituted or substituted by one or more L^{B}, a methylidene group which may be unsubstituted or substituted by one or more L^{B}, or a group which may be unsubstituted or substituted by one or more L^{B} and are selected from the formula (B-1-8) and the formula (B-1-10).

Further, a group represented by the formula (B-1-3) preferably represents a group selected from formula (B-1-3-1) to formula (B-1-3-7) below,

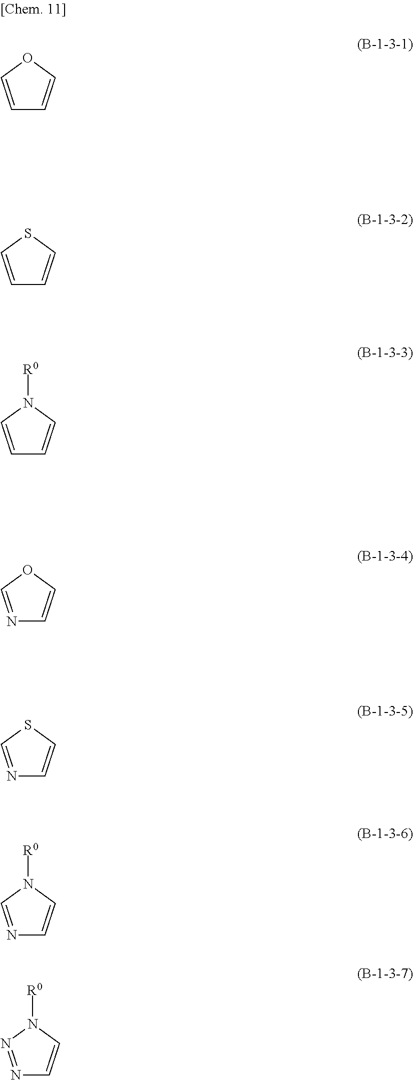

(in the formulae, a ring structure may have a bond at any position, R⁰ represents a hydrogen atom or an alkyl group having 1 to 20 carbon atoms, and these groups may be unsubstituted or substituted by one or more substituents L^{B}).

A group represented by the formula (B-1-4) preferably represents a group selected from formula (B-1-4-1) to formula (B-1-4-8) below,

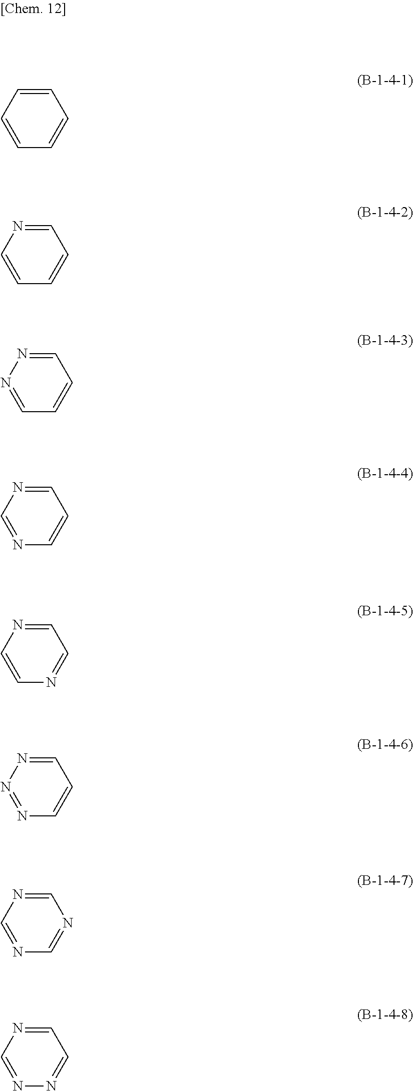

(in the formulae, a ring structure may have a bond at any position, and these groups may be unsubstituted or substituted by one or more substituents L^{B}).

A group represented by the formula (B-1-5) preferably represents a group selected from formula (B-1-5-1) to formula (B-1-5-6) below,

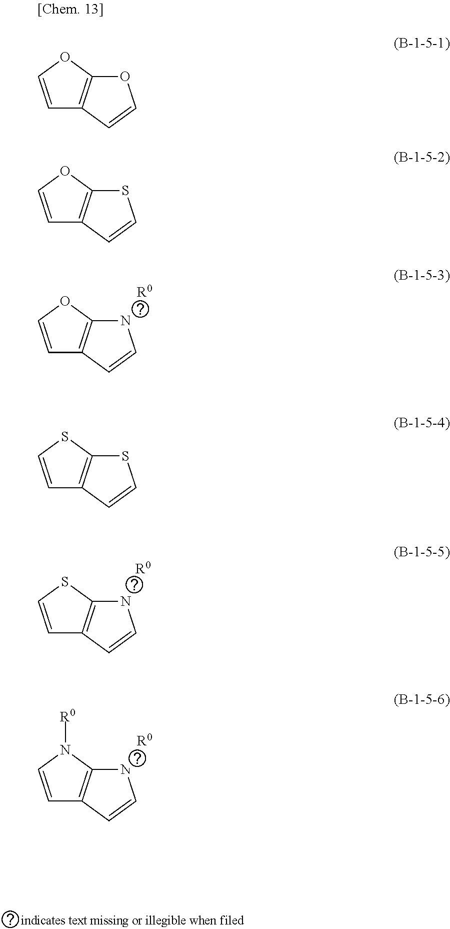

(in the formulae, a ring structure may have a bond at any position, R⁰ represents a hydrogen atom or an alkyl group having 1 to 20 carbon atoms, and these groups may be unsubstituted or substituted by one or more substituents L^{B}).

A group represented by the formula (B-1-6) preferably represents a group selected from formula (B-1-6-1) to formula (B-1-6-9) below,

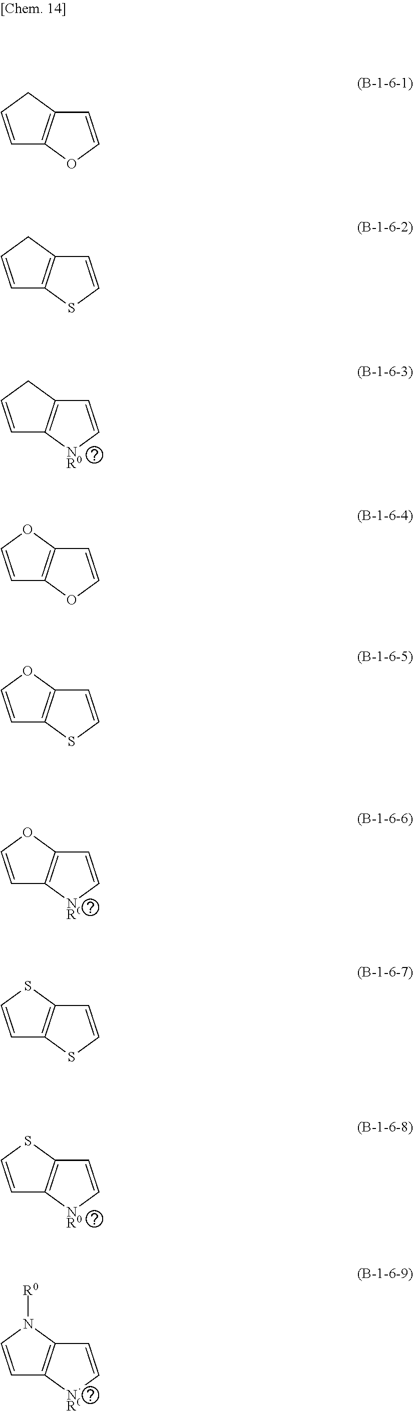

(in the formulae, a ring structure may have a bond at any position, R⁰ represents a hydrogen atom or an alkyl group having 1 to 20 carbon atoms, and these groups may be unsubstituted or substituted by one or more substituents L^{B}).

A group represented by the formula (B-1-7) preferably represents a group selected from formula (B-1-7-1) to formula (B-1-7-12) below,

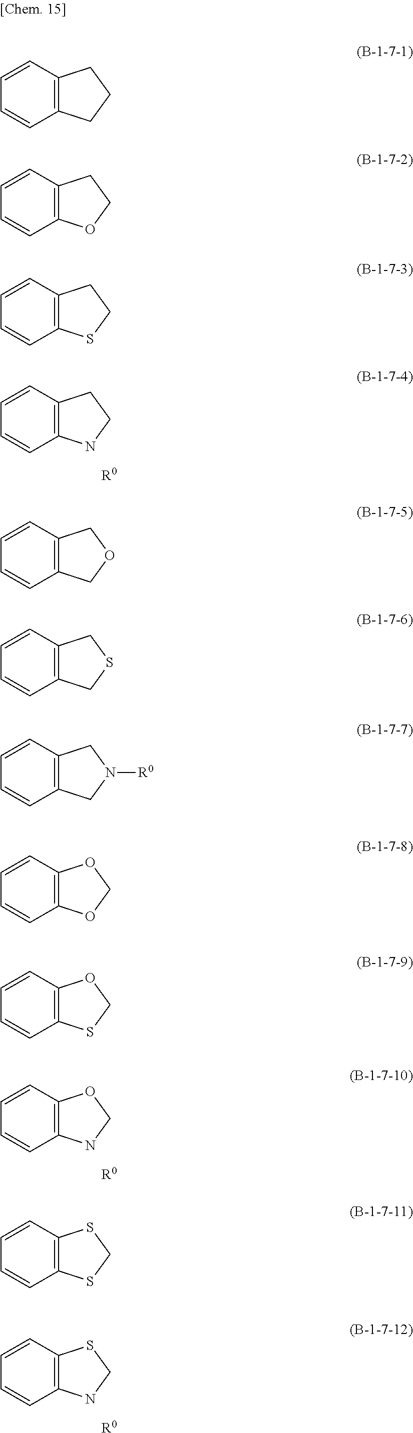

(in the formulae, a ring structure may have a bond at any position, R⁰ represents a hydrogen atom or an alkyl group having 1 to 20 carbon atoms, and these groups may be unsubstituted or substituted by one or more substituents L^{B}).

A group represented by the formula (B-1-8) preferably represents a group selected from formula (B-1-8-1) to formula (B-1-8-8) below,

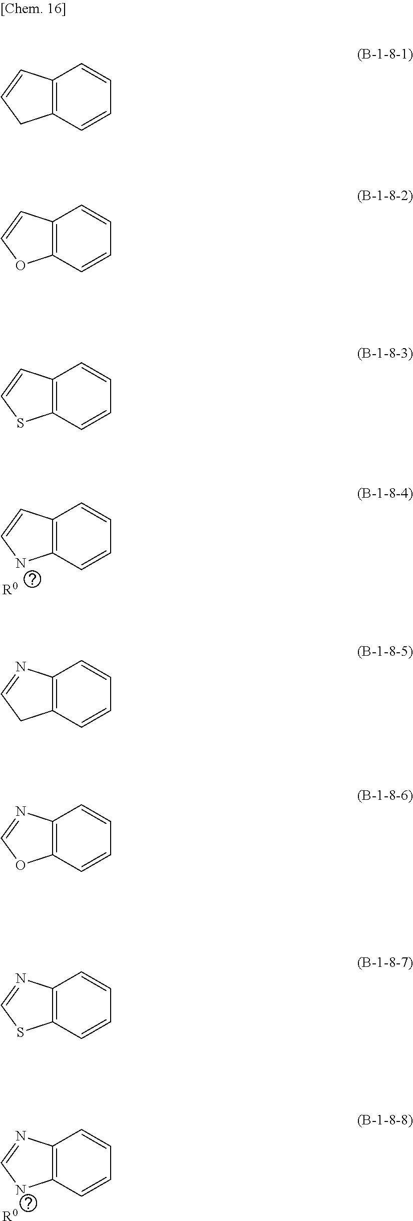

(in the formulae, a ring structure may have a bond at any position, R⁰ represents a hydrogen atom or an alkyl group having 1 to 20 carbon atoms, and these groups may be unsubstituted or substituted by one or more substituents L^{B}), and from the viewpoint of easy availability of raw materials and easy synthesis, the group more preferably represents a group selected from the formula (B-1-8-6), the formula (B-1-8-7), and the formula (B-1-8-8).

A group represented by the formula (B-1-10) preferably represents a group selected from formula (B-1-10-1) to formula (B-1-10-19) below,

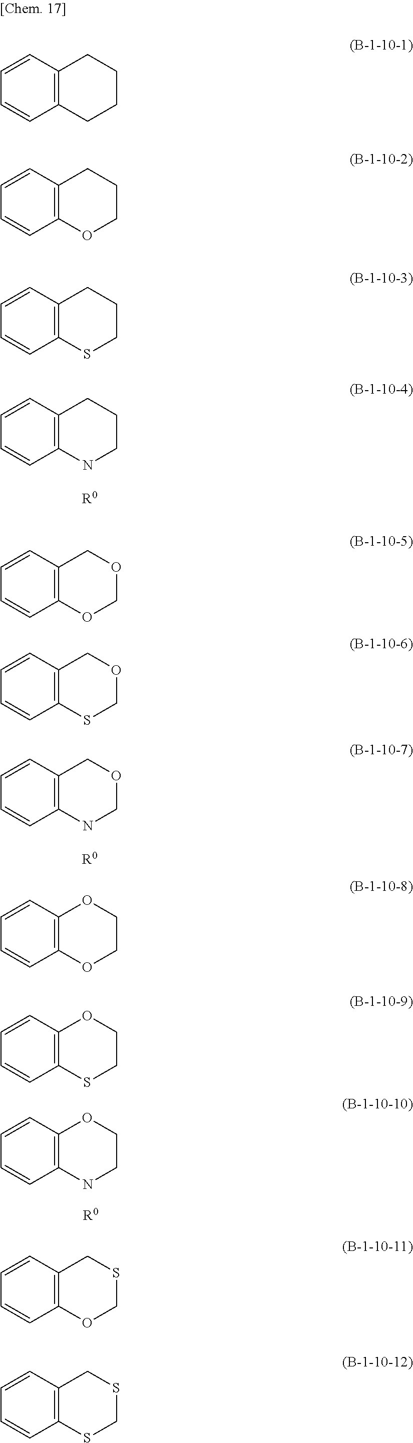

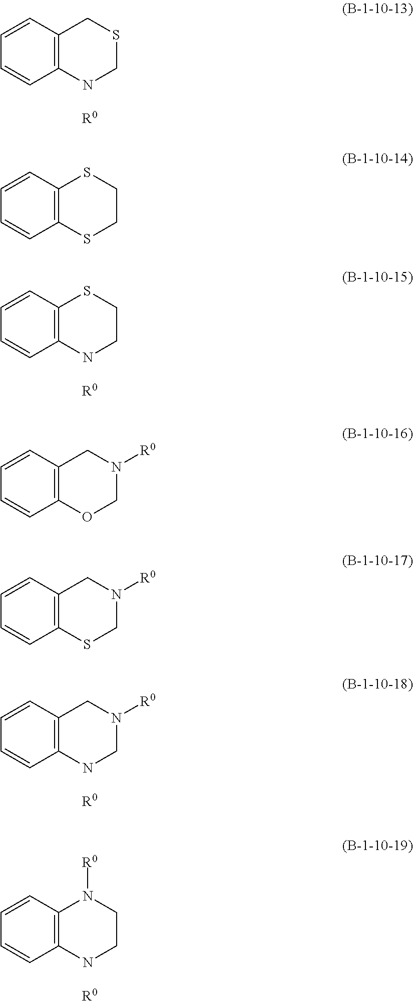

(in the formulae, a ring structure may have a bond at any position, R⁰ represents a hydrogen atom or an alkyl group having 1 to 20 carbon atoms, and these groups may be unsubstituted or substituted by one or more substituents L^{B}), and from the viewpoint of easy availability of raw materials and easy synthesis, the group more preferably represents a group selected from the formula (B-1-10-1), the formula (B-1-10-2), and the formula (B-1-10-3).

A group represented by the formula (B-1-11) preferably represents a group selected from formula (B-1-11-1) to formula (B-1-11-7) below,

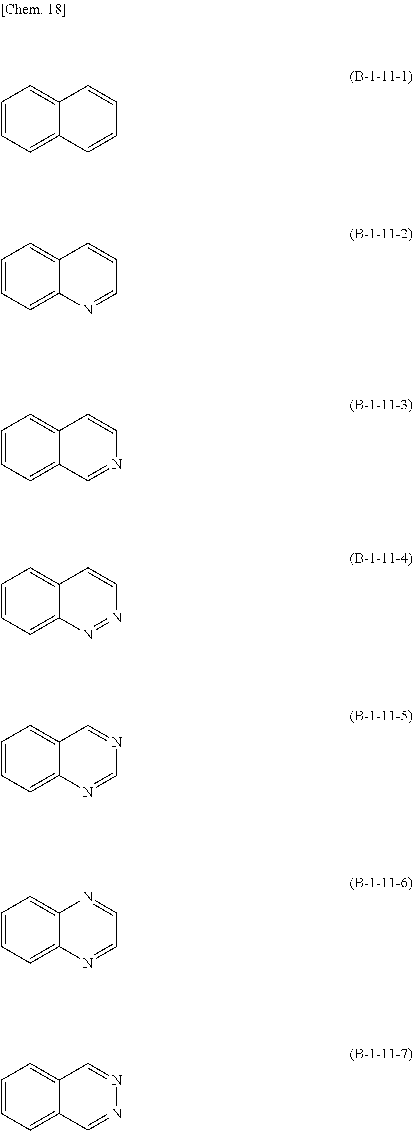

(in the formulae, a ring structure may have a bond at any position, R⁰ represents a hydrogen atom or an alkyl group having 1 to 20 carbon atoms, and these groups may be unsubstituted or substituted by one or more substituents L^{B}).

A group represented by the formula (B-1-12) preferably represents a group selected from formula (B-1-12-1) to formula (B-1-12-4) below,

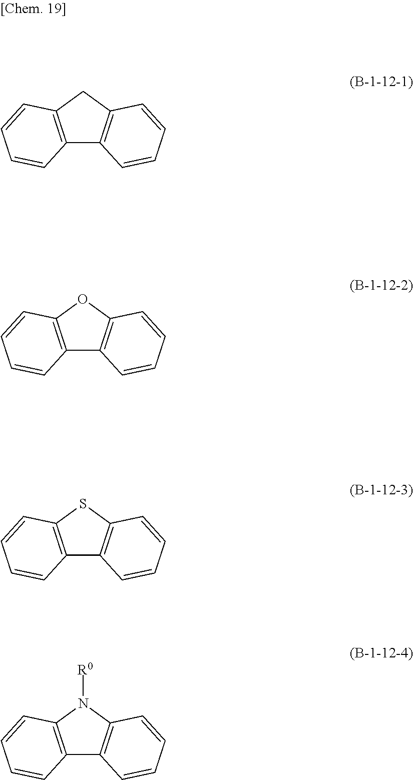

(in the formulae, a ring structure may have a bond at any position, R⁰ represents a hydrogen atom or an alkyl group having 1 to 20 carbon atoms, and these groups may be unsubstituted or substituted by one or more substituents L^{B}).

A group represented by the formula (B-1-13) preferably represents a group selected from formula (B-1-13-1) to formula (B-1-13-10) below,

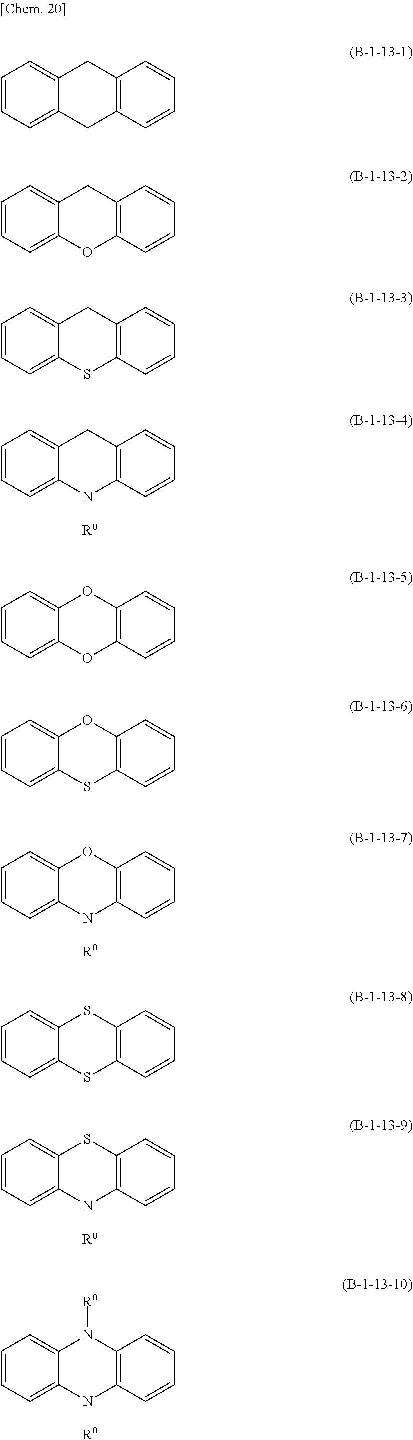

(in the formulae, a ring structure may have a bond at any position, R⁰ represents a hydrogen atom or an alkyl group having 1 to 20 carbon atoms, and these groups may be unsubstituted or substituted by one or more substituents L^{B}).

A group represented by the formula (B-1-17) preferably represents a group selected from formula (B-1-17-1) to formula (B-1-17-16) below,

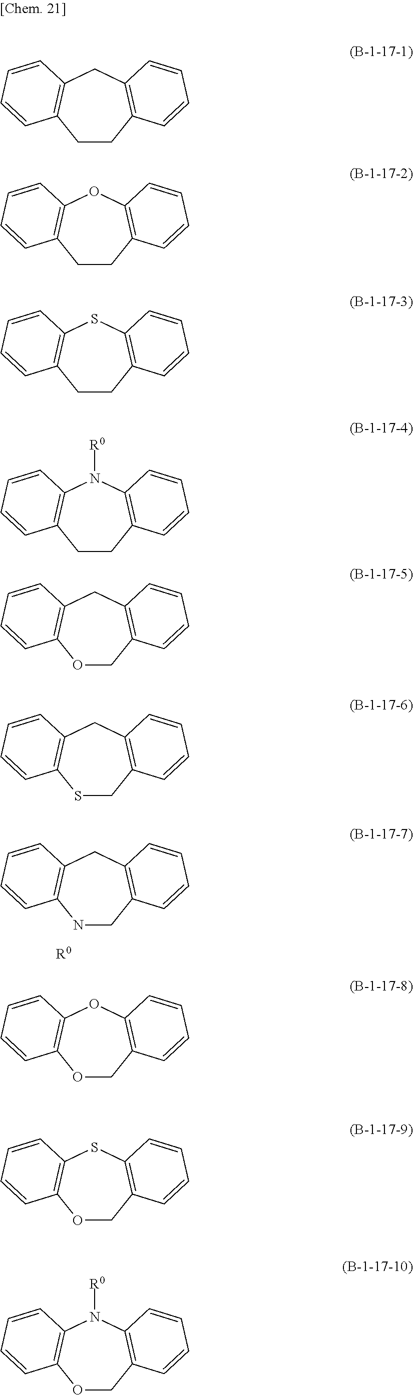

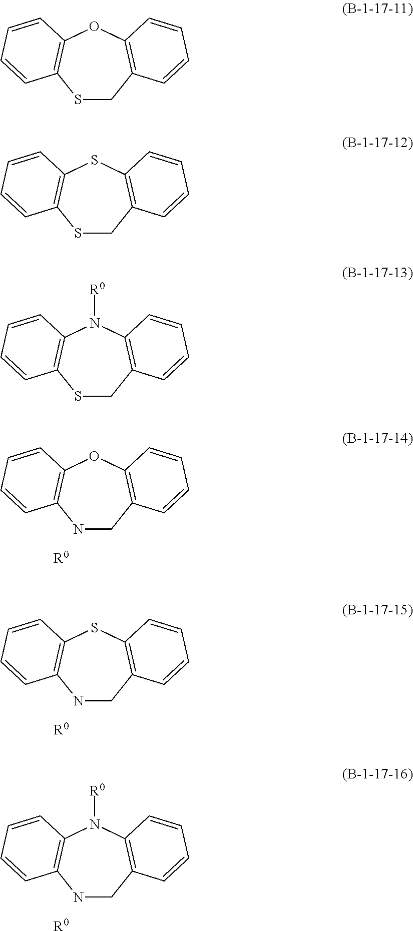

(in the formulae, a ring structure may have a bond at any position, R⁰ represents a hydrogen atom or an alkyl group having 1 to 20 carbon atoms, and these groups may be unsubstituted or substituted by one or more substituents L^{B}).

A group represented by the formula (B-1-18) preferably represents a group selected from formula (B-1-18-1) to formula (B-1-18-4) below,

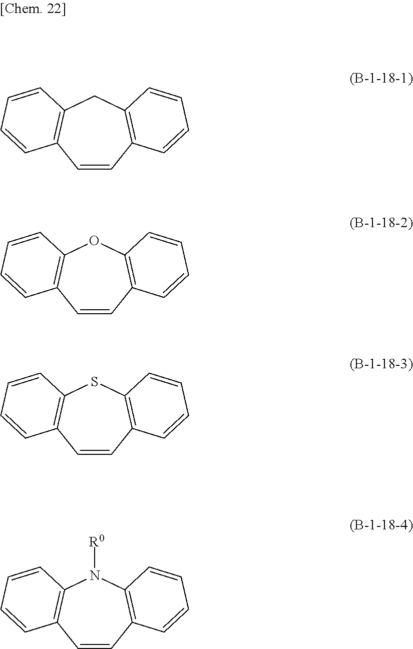

(in the formulae, a ring structure may have a bond at any position, R⁰ represents a hydrogen atom or an alkyl group having 1 to 20 carbon atoms, and these groups may be unsubstituted or substituted by one or more substituents L^{B}).

A group represented by the formula (B-1-19) preferably represents a group selected from formula (B-1-19-1) to formula (B-1-19-16) below,

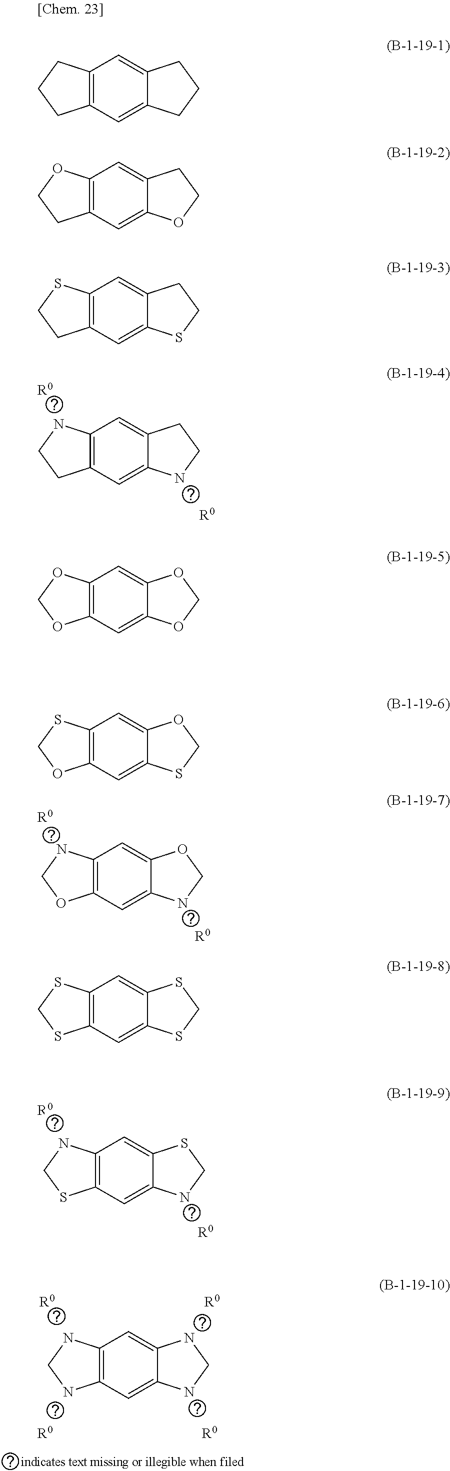

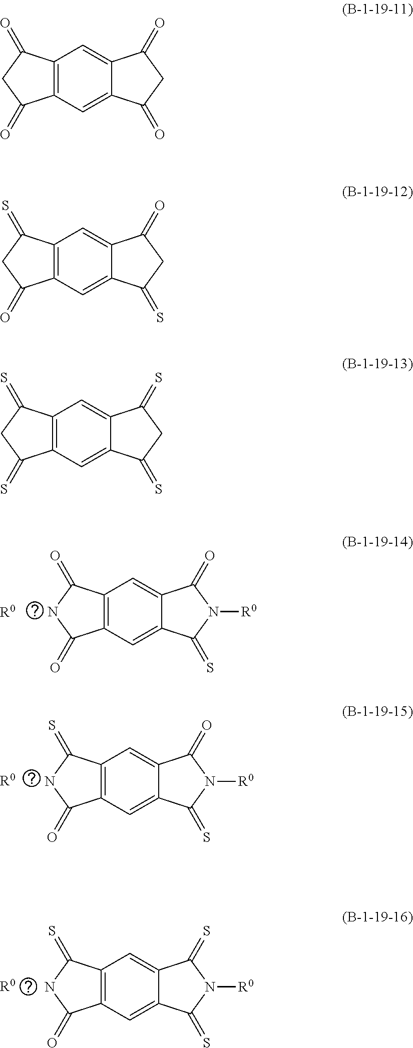

(in the formulae, a ring structure may have a bond at any position, R⁰ represents a hydrogen atom or an alkyl group having 1 to 20 carbon atoms, and these groups may be unsubstituted or substituted by one or more substituents L^{B}).

A group represented by the formula (B-1-20) preferably represents a group selected from formula (B-1-20-1) to formula (B-1-20-12) below,

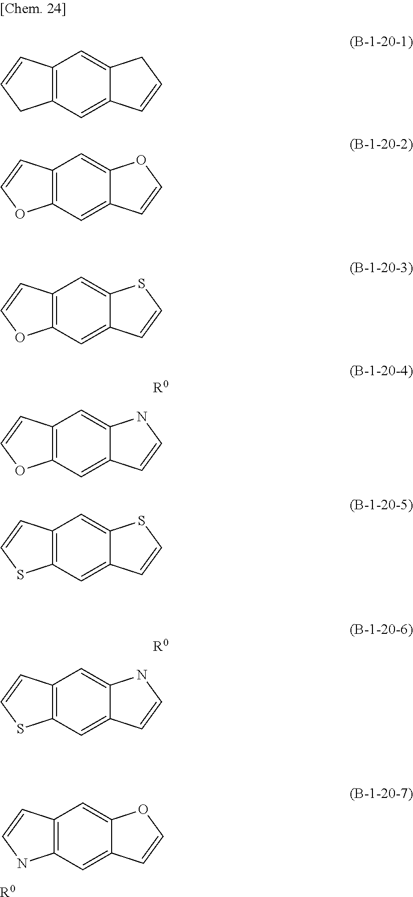

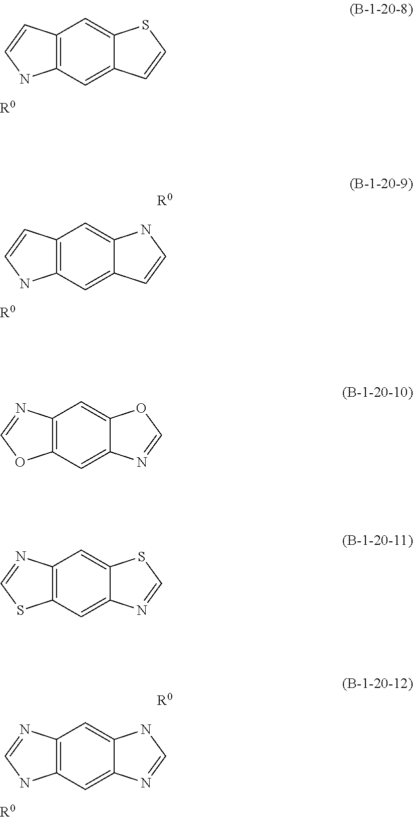

(in the formulae, a ring structure may have a bond at any position, R⁰ represents a hydrogen atom or an alkyl group having 1 to 20 carbon atoms, and these groups may be unsubstituted or substituted by one or more substituents L^{B}).

A group represented by the formula (B-1-21) preferably represents a group selected from formula (B-1-21-1) to formula (B-1-21-13) below,

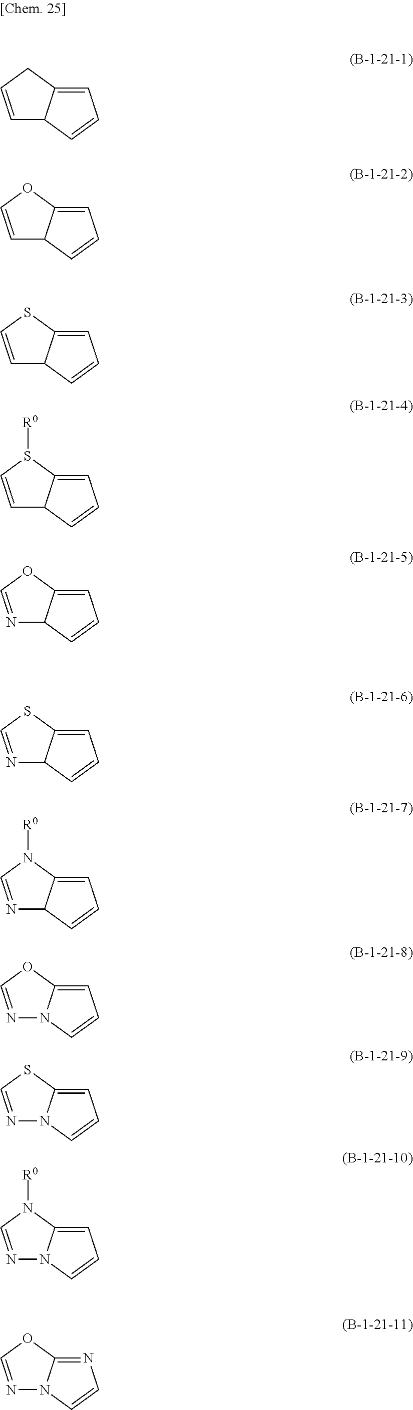

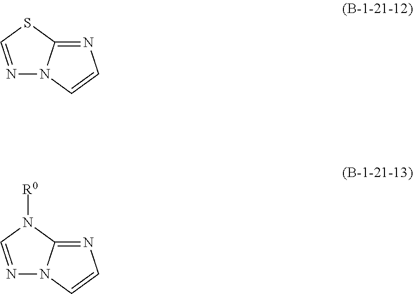

(in the formulae, a ring structure may have a bond at any position, R⁰ represents a hydrogen atom or an alkyl group having 1 to 20 carbon atoms, and these groups may be unsubstituted or substituted by one or more substituents L^{B}).

B² represents a single bond, a double bond, or a divalent cyclic hydrocarbon group which may be unsubstituted or substituted by one or more L^{B}, and from the viewpoint of liquid crystallinity, easy availability of raw materials and easy synthesis, B² preferably represents a single bond, a double bond, or a group selected from formula (B-2-1) to formula (B-2-21) below,

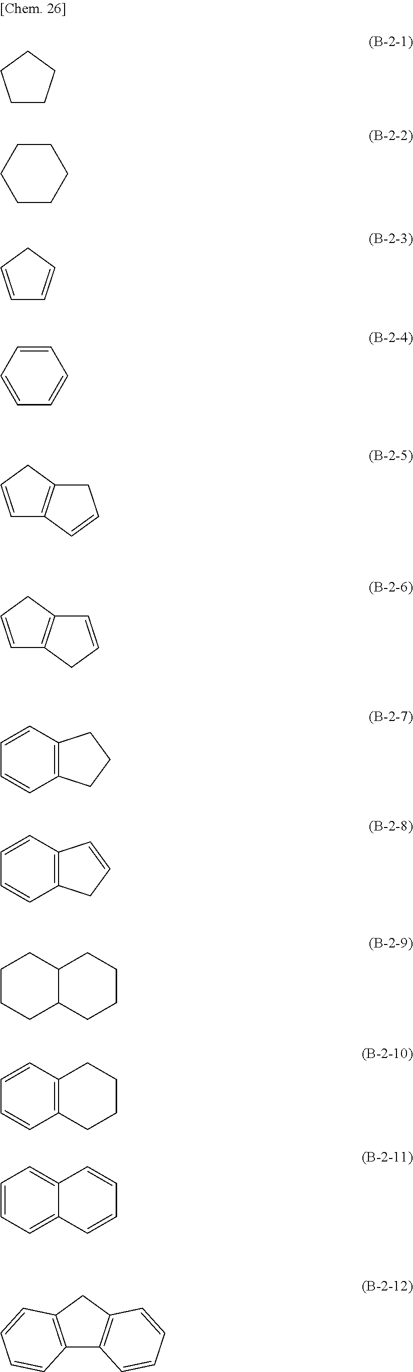

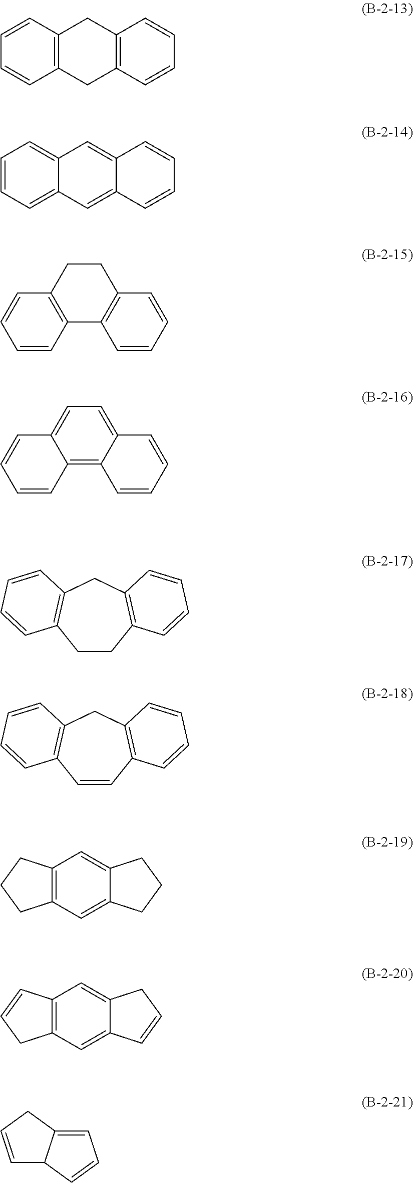

(in the formulae, a ring structure may have a bond at any desired position, any desired —CH═ may be each independently replaced by —N═, —CH₂— may be each independently substituted by —O—, —S—, —NR⁰— (wherein R⁰ represents a hydrogen atom or an alkyl group having 1 to 20 carbon atoms), —CS—, or —CO— without containing a —O—O— bond, and these groups may be unsubstituted or substituted by one or more substituents L^{B}), and B² more preferably represents a single bond, a double bond, or a group selected from formula the formula (B-2-3) and the formula (B-2-4).

V¹ and V² each represent a single bond, a double bond, or a divalent bonding group, and from the viewpoint of liquid crystallinity, easy availability of raw materials, and easy synthesis, V¹ and V² preferably each represent a single bond, a double bond, or a group selected from formula (V-1) to formula (V-15) below,

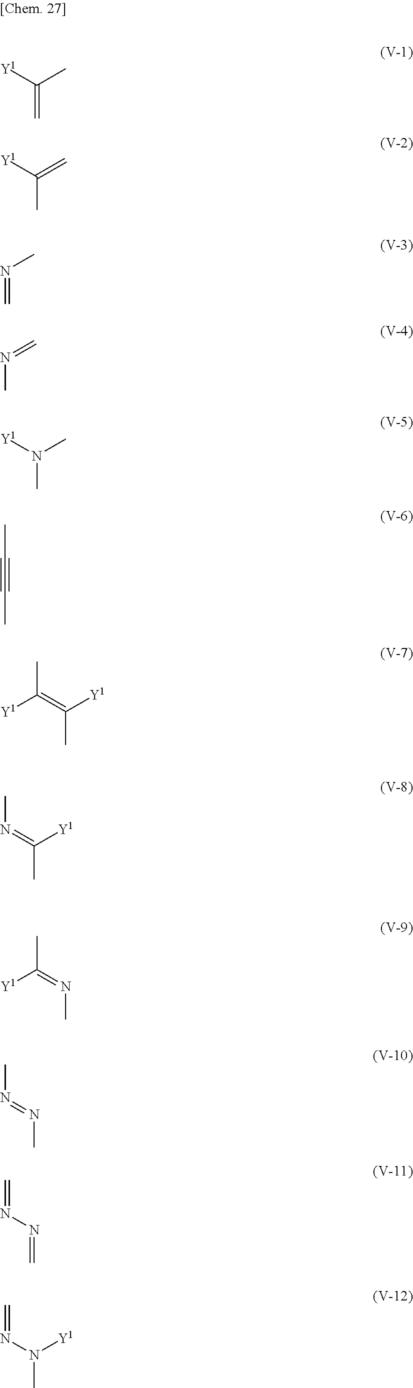

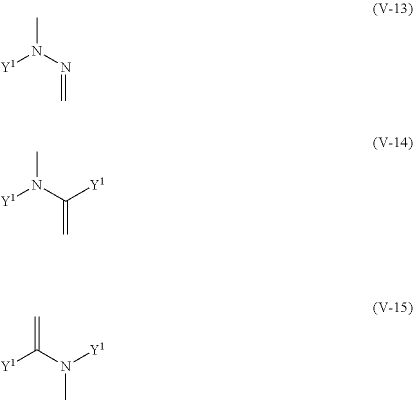

(in the formulae, Y¹ represents a hydrogen atom, a fluorine atom, a chlorine atom, a bromine atom, an iodine atom, a pentafluorosulfuranyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a linear or branched alkyl group having 1 to 20 carbon atoms in which one —CH₂— or two or more unadjacent —CH₂— may be each independently substituted by —O—, —S—, —CO—, —COO—, —OCO—, —CO—S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, or —C≡C—, any hydrogen atom in the alkyl group may be substituted by a fluorine atom, and when a plurality of Y¹ are present, they may be the same or different, or Y¹ may represent a group represented by P^{Y}—(S^{Y}—X^{Y})_{kY}—, wherein P^{Y} represents a polymerizable group; S^{Y} represents a spacer group or a single bond, and when a plurality of S^{Y} are present, they may be the same or different; X^{Y} represents —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —N═N—, —CH═N—N═CH—, —CF═CF—, —C≡C—, or a single bond, and when a plurality of X are present, they may be the same or different (wherein P^{Y}—(S^{Y}—X^{Y})_{kY}— does not contain an —O—O— bond); and kY represents an integer of 0 to 10), —O—, —S—, —OCH₂—, —CH₂O—, —CO—, —CH₂—, —COO—, —OCO—, —CO—S—, —S—CO—, —O—CO—O—, —CO—NH—, —NH—CO—, —CS—NH—, —NH—CS—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —CH₂CH₂—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, and —CH₂—OCO—; V¹ and V² more preferably each independently represent a single bond, a double bond, or a group selected from the formula (V-2), the formula (V-5) to the formula (V-15), —CS—NH—, and —NH—CS—, V¹ and V² even more preferably each independently represent a single bond or a group selected from the formula (V-2), the formula (V-5), the formula (V-6), the formula (V-7), the formula (V-8), and the formula (V-13), V¹ and V² even more preferably each independently represent a single bond or a group selected from the formula (V-5), the formula (V-6), and the formula (V-7).

Further, n represents an integer of 0 to 10, and from the viewpoint of liquid crystallinity, easy availability of raw materials, and easy synthesis, n preferably represents an integer of 0 to 5 and more preferably represents 0, 1, 2, or 3.

From the viewpoint of liquid crystallinity, easy availability of raw materials, and easy synthesis, L preferably represents a fluorine atom, a chlorine atom, a pentafluorosulfuranyl group, a nitro group, a cyano group, a methylamino group, a dimethylamino group, a diethylamino group, a diisopropylamino group, or a linear or branched alkyl group having 1 to 20 carbon atoms in which any hydrogen atom may be substituted by a fluorine atom, and one —CH₂— or two or more unadjacent —CH₂— may be each independently substituted by a group selected from —O—, —S—, —CO—, —COO—, —OCO—, —O—CO—O—, —CH═CH—, —CF═CF—, and —C≡C—, and more preferably represents a fluorine atom, a cyano group, or a linear or branched alkyl group having 1 to 12 carbon atoms in which any hydrogen atom may be substituted by a fluorine atom, and one —CH₂— or two or more unadjacent —CH₂— may be each independently substituted by a group selected from —O—, —COO—, and —OCO—.

From the viewpoint of easy synthesis, easy availability of raw materials, and liquid crystallinity, L^{B} preferably represents a fluorine atom, a chlorine atom, a nitro group, a cyano group, a methylamino group, a dimethylamino group, a diethylamino group, a diisopropylamino group, or a linear or branched alkyl group having 1 to 20 carbon atoms in which any hydrogen atom may be substituted by a fluorine atom, and one —CH₂— or two or more unadjacent —CH₂— may be each independently substituted by —O—, —S—, —CO—, —COO—, —OCO—, —O—CO—O—, —CH═CH—, —CF═CF—, or —C≡C—, preferably represents a fluorine atom, a chlorine atom, a nitro group, a cyano group, a methylamino group, a dimethylamino group, or a linear or branched alkyl group having 1 to 20 carbon atoms in which any hydrogen atom may be substituted by a fluorine atom, and one —CH₂— or two or more unadjacent —CH₂— may be each independently substituted by —O—, —S—, or —CO—, more preferably represents a fluorine atom, a chlorine atom, a nitro group, a cyano group, a methylamino group, a dimethylamino group, or a linear alkyl group having 1 to 10 carbon atoms in which any hydrogen atom may be substituted by a fluorine atom, and one —CH₂— or two or more unadjacent —CH₂— may be each independently substituted by —O—, and even more preferably represents a fluorine atom, a chlorine atom, a nitro group, a cyano group, a dimethylamino group, or a linear alkyl group having 1 or 2 carbon atoms in which any hydrogen atom may be substituted by a fluorine atom, and one —CH₂— or two or more unadjacent —CH₂— may be each independently substituted by —O—.

L^{T} represents a fluorine atom, a chlorine atom, a bromine atom, an iodine atom, a pentafluorosulfuranyl group, a nitro group, a cyano group, an isocyano group, an amino group, a hydroxyl group, a mercapto group, a methylamino group, a dimethylamino group, a diethylamino group, a diisopropylamino group, a trimethylsilyl group, a dimethylsilyl group, a thioisocyano group, or a linear or branched alkyl group having 1 to 20 carbon atoms in which one —CH₂— or two or more unadjacent —CH₂— may be each independently substituted by —O—, —S—, —CO—, —COO—, —OCO—, —CO—S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, or —C≡C—, any hydrogen atom in the alkyl group may be substituted by a fluorine atom, and when a plurality of L^{T} are present, they may be the same or different. From the viewpoint of easy synthesis, easy availability of raw materials, and liquid crystallinity, L^{T} preferably represents a fluorine atom, a chlorine atom, a nitro group, a cyano group, a methylamino group, a dimethylamino group, or a linear or branched alkyl group having 1 to 20 carbon atoms in which any hydrogen atom in the group may be substituted by a fluorine atom, and one —CH₂— or two or more unadjacent —CH₂— may be each independently substituted by —O—, —S—, —CO—, —COO—, —OCO—, —O—CO—O—, —CH═CH—, —CF═CF—, or —C≡C—, preferably represents a fluorine atom, a chlorine atom, a nitro group, a cyano group, a methylamino group, a dimethylamino group, a diethylamino group, a diisopropylamino group, or a linear or branched alkyl group having 1 to 20 carbon atoms in which any hydrogen atom in the group may be substituted by a fluorine atom, and one —CH₂— or two or more unadjacent —CH₂— may be each independently substituted by —O—, —S—, or —CO—, more preferably represents a fluorine atom, a chlorine atom, a nitro group, a cyano group, a methylamino group, a dimethylamino group, or a linear alkyl group having 1 to 10 carbon atoms in which any hydrogen atom in the group may be substituted by a fluorine atom, and one —CH₂— or two or more unadjacent —CH₂— may be each independently substituted by —O—, and even more preferably represents a fluorine atom, a chlorine atom, a nitro group, a cyano group, a dimethylamino group, or a linear alkyl group having 1 or 2 carbon atoms in which any hydrogen atom in the group may be substituted by a fluorine atom, and one —CH₂— or two or more unadjacent —CH₂— may be each independently substituted by —O—.

Further, k represents an integer of 0 to 8, and from the viewpoint of liquid crystallinity, easy availability of raw materials, and easy synthesis, k preferably represents an integer of 0 to 4, more preferably represents an integer of 0 to 2, even more preferably represents 0 or 1, and particularly preferably represents 1.

In addition, m1 and m2 each independently represent an integer of 0 to 5, and m1+m2 represents an integer of 1 to 5. From the viewpoint of liquid crystallinity, easy availability of raw materials, and solubility in solvents, m1 and m2 preferably each independently represent an integer of 1 to 4, more preferably represent an integer of 1 to 3, and particularly preferably represent 1 or 2. m1+m2 preferably represents an integer of 1 to 4, more preferably represents 2, 3, or 4, and particularly preferably represent 2 or 4.

Preferred examples of the compound represented by the general formula (I) include compounds represented by formula (I-1) to formula (I-10) below.

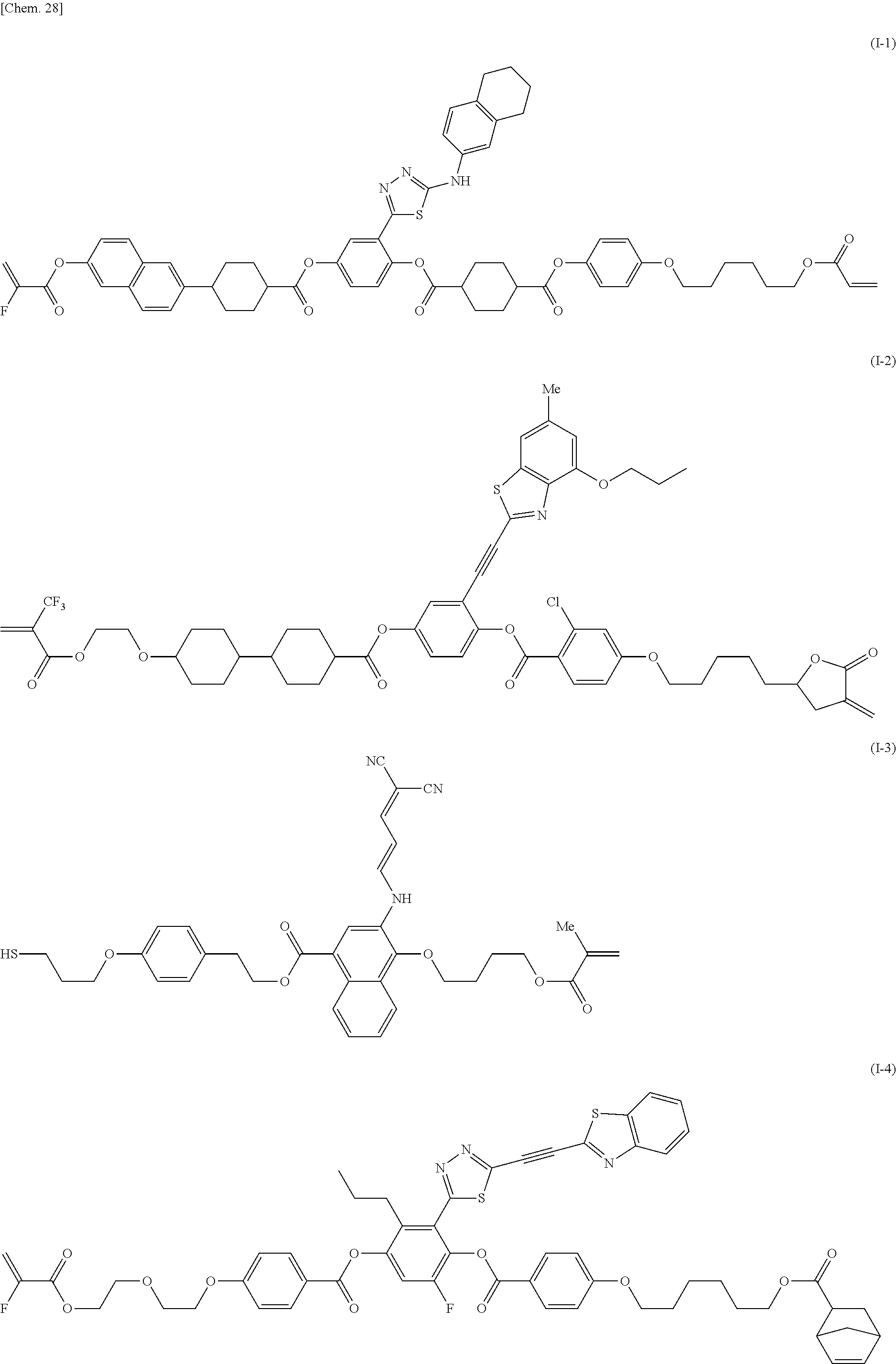

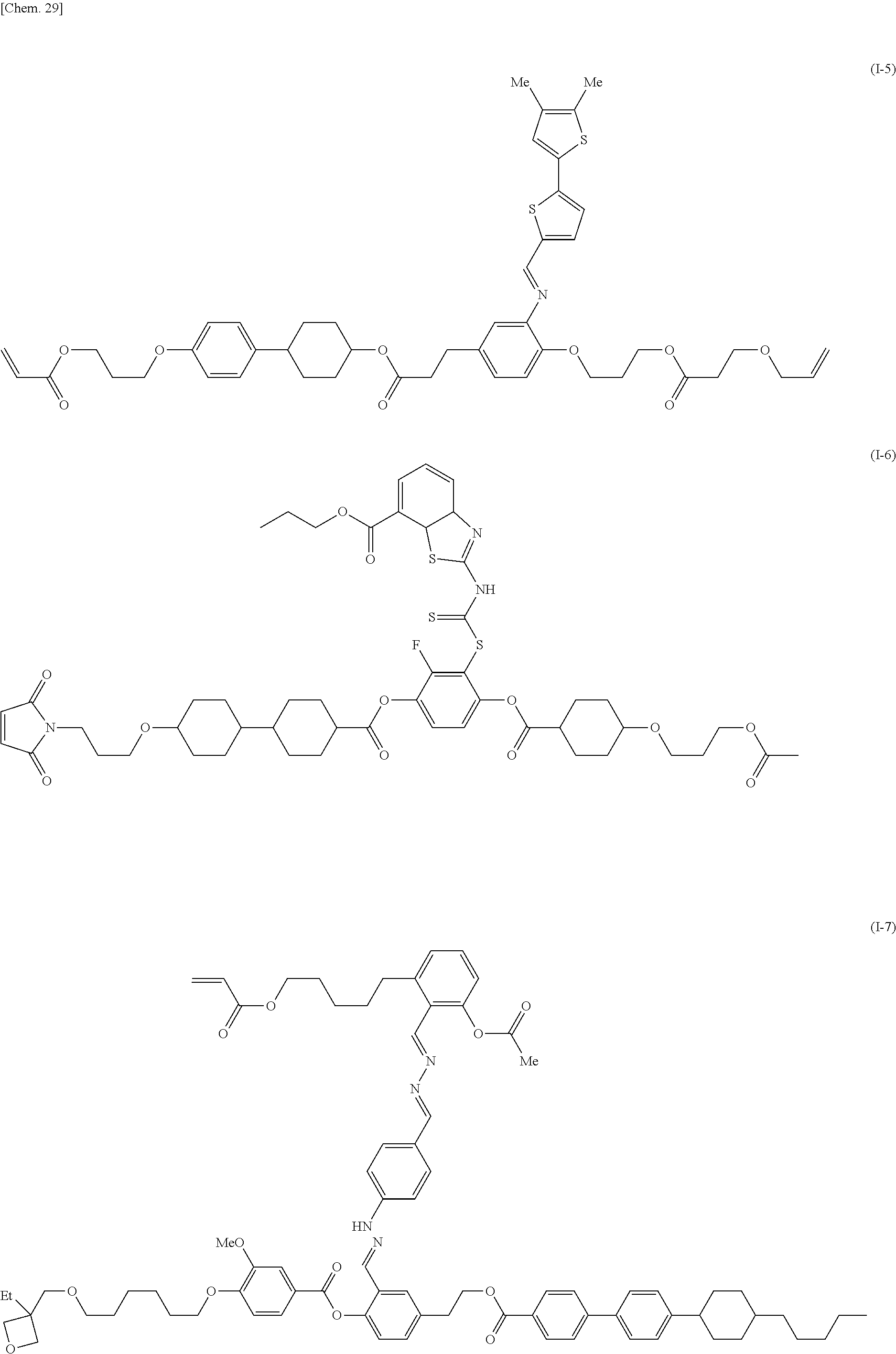

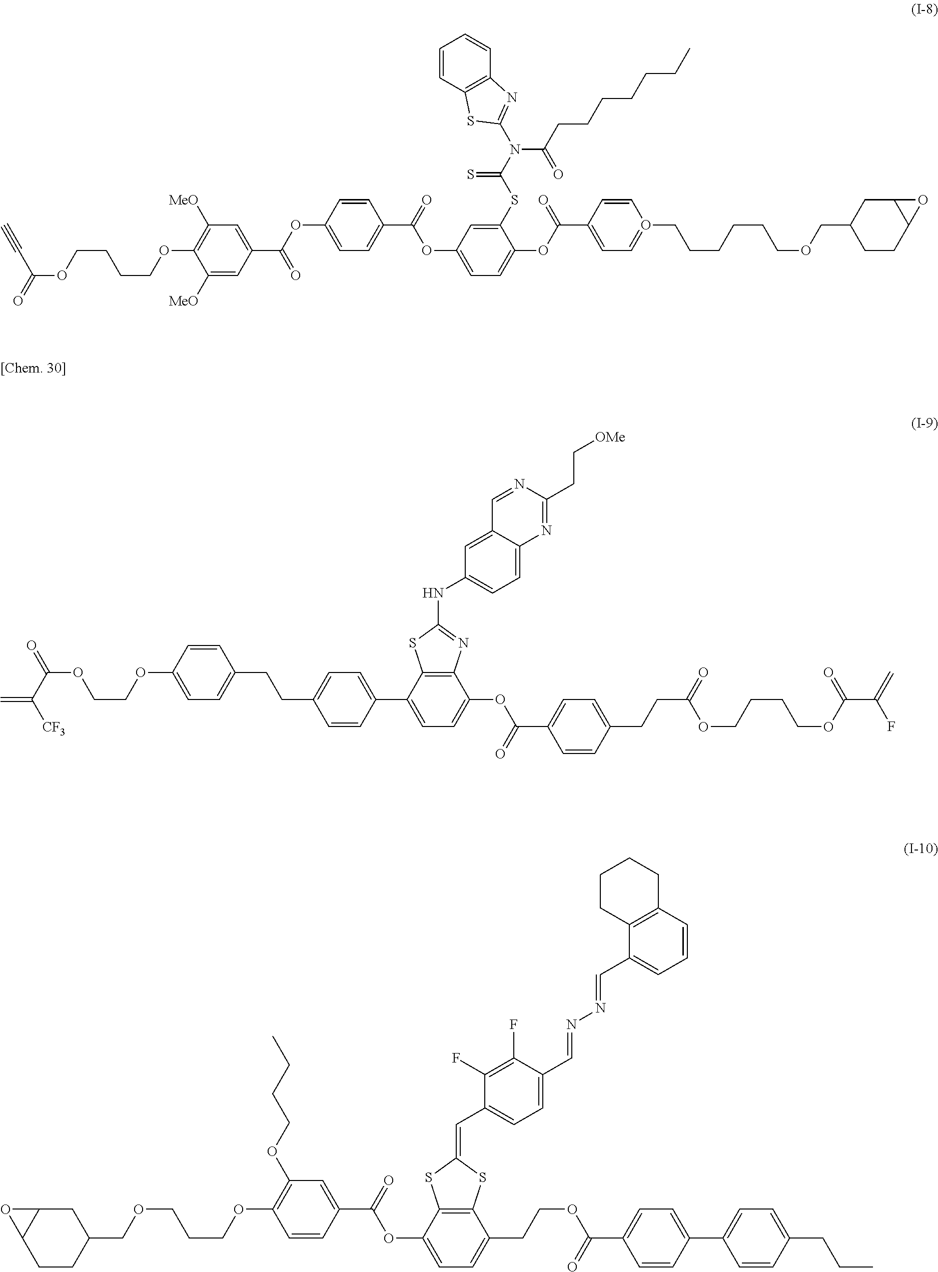

The compound, of the present invention is preferably used for a nematic liquid crystal composition, a smectic liquid crystal composition, a chiral smectic liquid crystal composition, and a cholesteric liquid crystal composition. A compound other than the compound of the present invention may be added to a liquid crystal composition using the reactive compound of the present invention.

Preferred examples of the other polymerizable compound used by being mixed with the polymerizable compound of the present invention include compounds represented by general formula (X-11) and/or

[Chem. 31]

P¹¹—S¹¹—X¹¹A¹¹-Z¹¹546 ₘ₁₁A¹²—X¹²—S¹²—P¹² (X-11)

general formula (X-12)

[Chem. 32]

P¹³—S¹³—X¹³A¹³-Z¹²ₘ₁₂A¹⁴—R¹¹ (X-12)

(in the formulae, P¹¹, P¹², and P¹³ each independently represent a polymerizable group; S¹¹, S¹², and S¹³ each independently represent a single bond or an alkylene group having 1 to 20 carbon atoms, in which one —CH₂— or two or more unadjacent —CH₂— may be each independently substituted by —O—, —COO—, —OCO—, or —OCOO—; X¹, X², and X³ each independently represent —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —CF═CF—, —C≡C—, or a single bond; Z¹¹ and Z¹² each independently represent —O—, —S—, —OCH₂—, —CH₂O—, —COO—, —OCO—, —CO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH₂CH₂—, —CH₂CF₂—, —CF₂CH₂—, —CF₂CF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —CF═CF—, —C≡C—, or a single bond; A¹¹, A¹², A¹³, and A¹⁴ each independently represent a 1,4-phenylene group, a 1,4-cyclohexylene group, a pyridine-2,5-diyl group, a pyrimidine-2,5-diyl group, a naphthalene-2,6-diyl group, a naphthalene-1,4-diyl group, a tetrahydronaphthalene-2,6-diyl group, or a 1,3-dioxane-2,5-diyl group, A¹¹, A¹², A¹³, and A¹⁴ may be each independently unsubstituted or substituted by an alkyl group, a halogenated alkyl group, an alkoxy group, a halogenated alkoxy group, a halogen atom, a cyano group, or a nitro group; R¹¹ represents a hydrogen atom, a fluorine atom, a chlorine atom, a bromine atom, an iodine atom, a pentafluorosulfuranyl group, a cyano group, a nitro group, an isocyano group, a thioisocyano group, or a linear or branched alkyl group having 1 to 20 carbon atoms in which one —CH₂— or two or more unadjacent —CH₂— may be each independently substituted by —O—, —S—, —CO—, —COO—, —OCO—, —CO—S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, or —C≡C—; and m11 and m12 each independently represent 0, 1, 2, or 3, when m11 and/or m12 represents 2 or 3, two or three of each of A¹¹, A¹³, Z¹¹ and/or Z¹² may be the same or different), and P¹¹, P¹², and P¹³ are each particularly preferably an acryl group or methacryl group. Specifically, the compound represented by the general formula (X-11) is preferably a compound represented by general formula (X-11a),

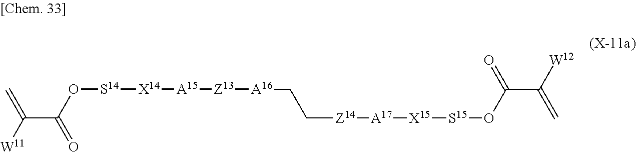

(in the formula, W¹¹ and W¹² each independently represent a hydrogen atom or a methyl group, S¹⁴ and S¹⁵ each independently represent an alkylene group having 2 to 18 carbon atoms; X¹⁴ and X¹⁵ each independently represent —O—, —COO—, —OCO—, or a single bond; Z¹³ and Z¹⁴ each independently represent —COO— or —OCO—; and A¹⁵, A¹⁶, and A¹⁷ each independently represent a 1,4-phenylene group which may be unsubstituted or substituted by a fluorine atom, a chlorine atom, a linear or branched alkyl group having 1 to 4 carbon atoms, or a linear or branched alkoxy group having 1 to 4 carbon atoms), and particularly preferred are compounds represented by formula (X-11a-1) to formula (X-11a-4) below,

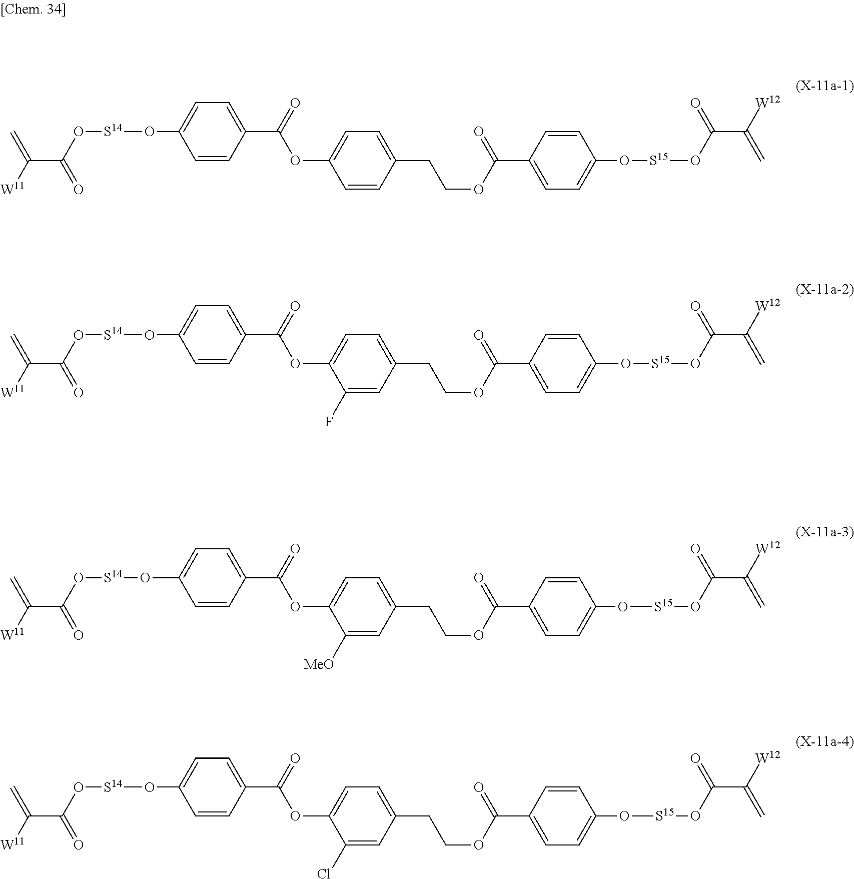

(in the formulae, W¹¹, W¹², S¹⁴ and S¹⁵ represents the same meanings as in the general formula (X-11a)). In the compounds represented by the formula (X-11a-1) to the formula (X-11a-4), compounds in which S¹⁴ and S¹⁵ each independently represent an alkylene group having 2 to 8 carbon atoms are particularly preferred.

Other preferred examples of the difunctional polymerizable compound include compounds represented by general formula (X-11b-1) to formula (X-11b-3) below

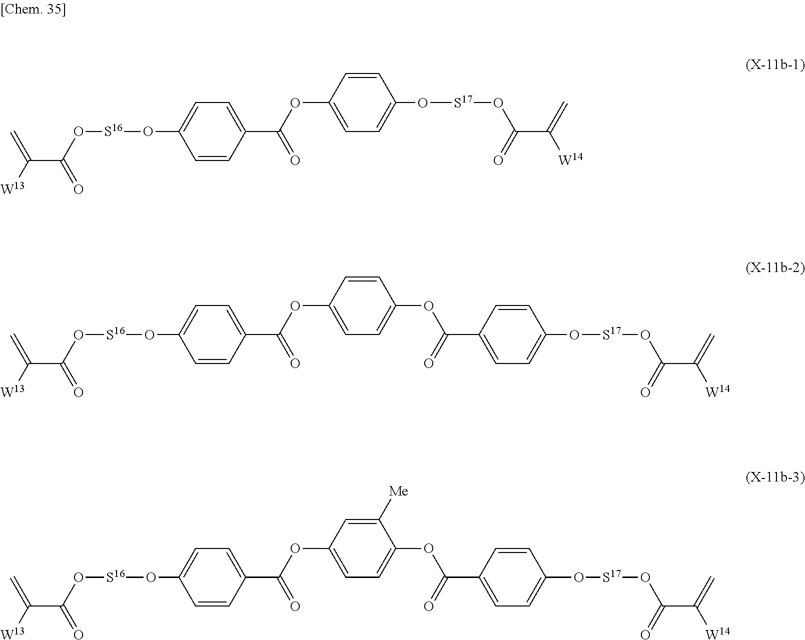

(in the formula, W¹³ and W¹⁴ each independently represent a hydrogen atom or a methyl group, and S¹⁶ and S¹⁷ each independently represent an alkylene group having 2 to 18 carbon atoms). In the formula (X-11b-1) to the formula (X-11b-3), compounds in which S¹⁶ and S¹⁷ each independently represent an alkylene group having 2 to 8 carbon atoms are particularly preferred.

Examples of a compound represented by the general formula (X-12) include compounds represented by general formula (X-12-1) to formula (X-12-7) below,

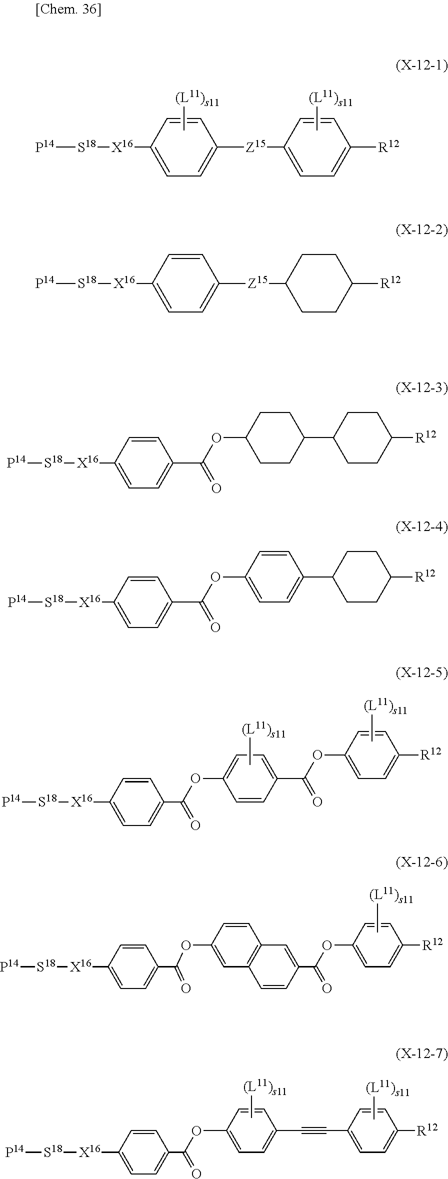

(in the formulae, P¹⁴ represents a polymerizable group; S¹⁸ represents a single bond or an alkylene group having 1 to 20 carbon atoms, in which one —CH₂— or two or more unadjacent —CH₂— may be each independently substituted by —O—, —COO—, —OCO—, or —O—CO—O—; X¹⁶ represents a single bond, —O—, —COO—, or —OCO—; Z¹⁵ represents a single bond, —COO—, or —OCO—; L¹¹ represents a fluorine atom, a chlorine atom, or a linear or branched alkyl group having 1 to 10 carbon atoms in which one —CH₂— or two or more unadjacent —CH₂— may be each independently substituted by —O—, —COO—, or —OCO—;s11 represents an integer of 0 to 4; and R¹² represents a hydrogen atom, a fluorine atom, a chlorine atom, a cyano group, a nitro group, or a linear or branched alkyl group having 1 to 20 carbon atoms in which one —CH₂— or two or more unadjacent —CH₂— may be each independently substituted by —O—, —S—, —CO—, —COO—, —OCO—, —CO—S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, or —C≡C—).

A polymerizable compound without showing liquid crystallinity can be added to the polymerizable liquid crystal composition containing the compound of the present invention in a degree that does not significantly impair the liquid crystallinity of the composition. Specifically, any compounds recognized as polymer-forming monomers or polymer-forming oligomers in this technical field can be used without particular limitation. Examples thereof include compounds described in “Photocuring Technology Data Book, Materials (monomers, oligomers, and photopolymerization initiators)” (supervised by Kunihiro Ichimura and Kiyomi Kato, Technonet).

In addition, the compound of the present invention can be polymerized without using a photopolymerization initiator, but a photopolymerization initiator may be added according to purpose. In this case, the concentration of the photopolymerization initiator is preferably 0.1% to 15% by mass, more preferably 0.2% to 10% by mass, and even more preferably 0.4% to 8% by mass relative to the compound of the present invention. Examples of the photopolymerization initiator include benzoin ethers, benzophenones, acetophenones, benzyl ketals, acylphosphine oxides, and the like. Specific examples of the photopolymerization initiator include 2-methyl-1-(4-methylthiophenyl)-2-morpholinopropan-1-one (IRGACURE 907), benzoic acid [1-[4-(phenylthio)benzoyl]heptylidene]amino ester (IRGACURE OXE 01), and the like. Examples of a thermopolymerization initiator include azo compounds, peroxides, and the like. Specific examples of the thermopolymerization initiator include 2,2′-azobis(4-methoxy-2,4-dimethylvaleronitrile), 2,2′-azobis(isobutyronitrile), and the like. In addition, one polymerization initiator may be used, or two or more polymerization initiators may be used in combination.

Also, in order to improve storage stability, a stabilizer can be added to the liquid crystal composition of the present invention. Examples of the stability which can be used include hydroquinones, hydroquinone monoalkyl ethers, tertiary butylcatechols, pyrogallols, thiophenols, nitro compounds, β-naphthylamines, β-naphthols, nitroso compounds, and the like. When the stabilizer is used, the adding amount is preferably within a range of 0.005% by mass to 1% by mass, more preferably 0.02% by mass to 0.8% by mass, and even more preferably 0.03% by mass to 0.5% by mass relative to the composition. In addition, one stabilizer may be used or two or more stabilizers may be used in combination. Preferred examples of the stabilizer include compounds represented by formula (X-13-1) to formula (X-13-35),

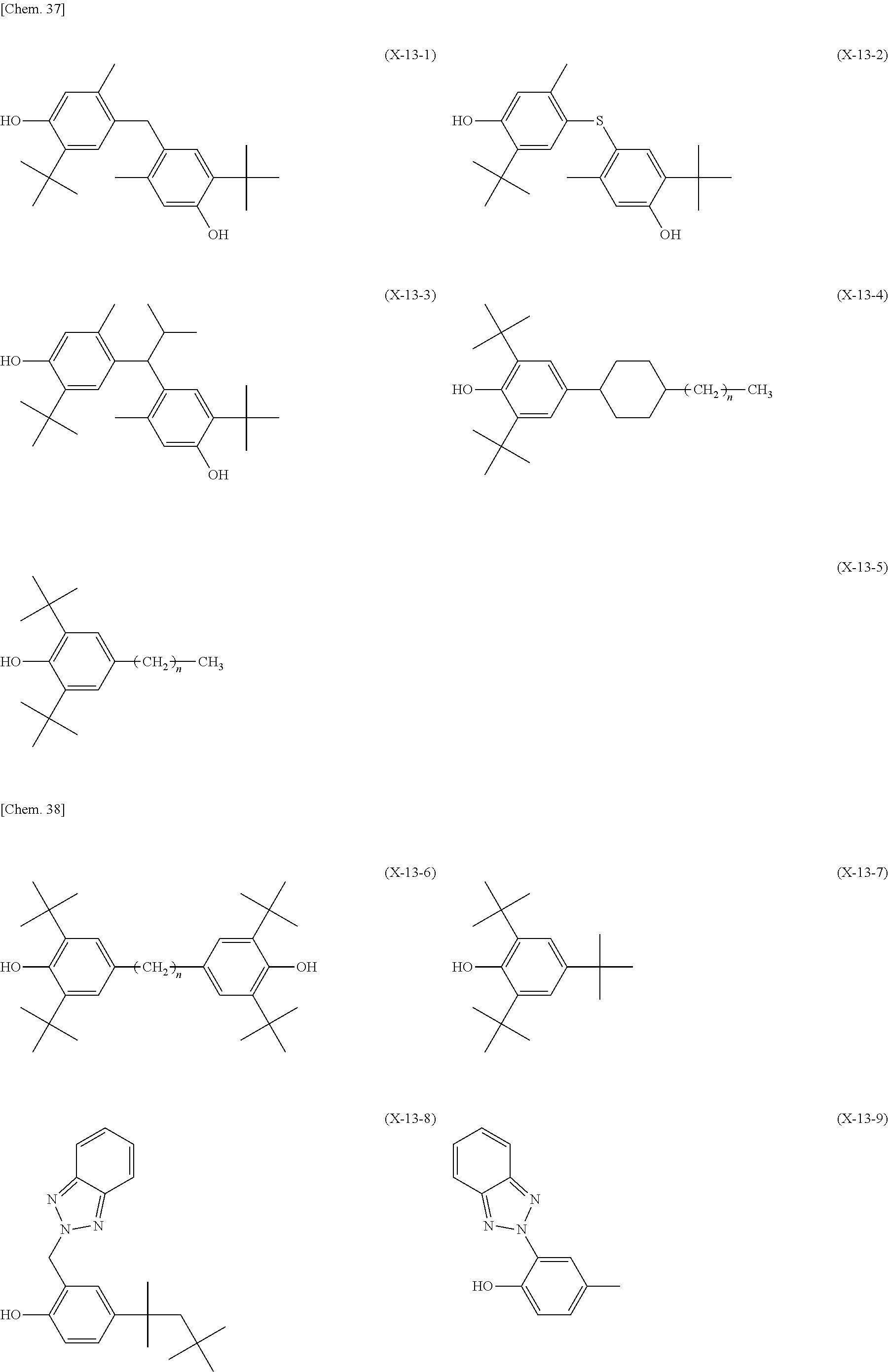

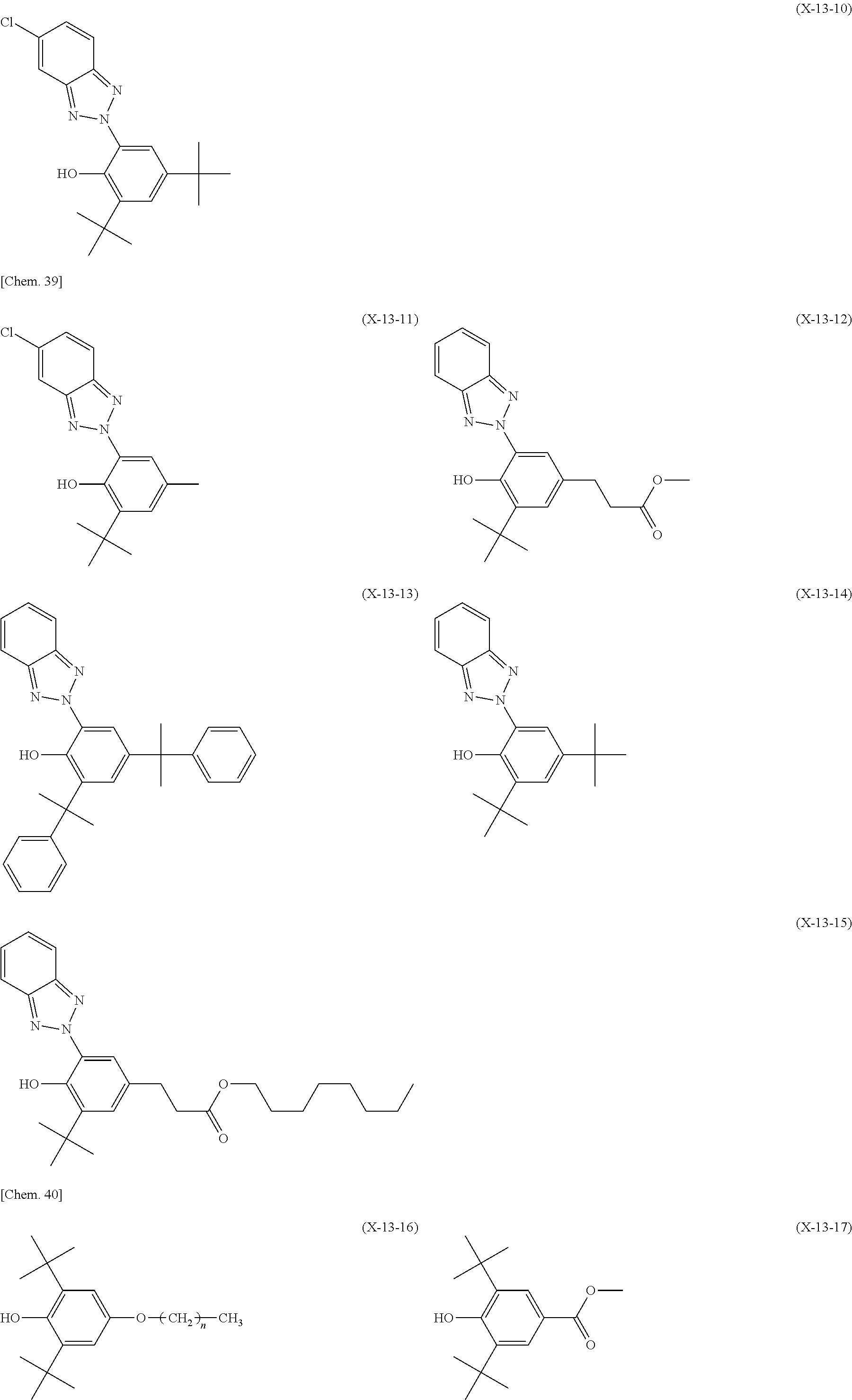

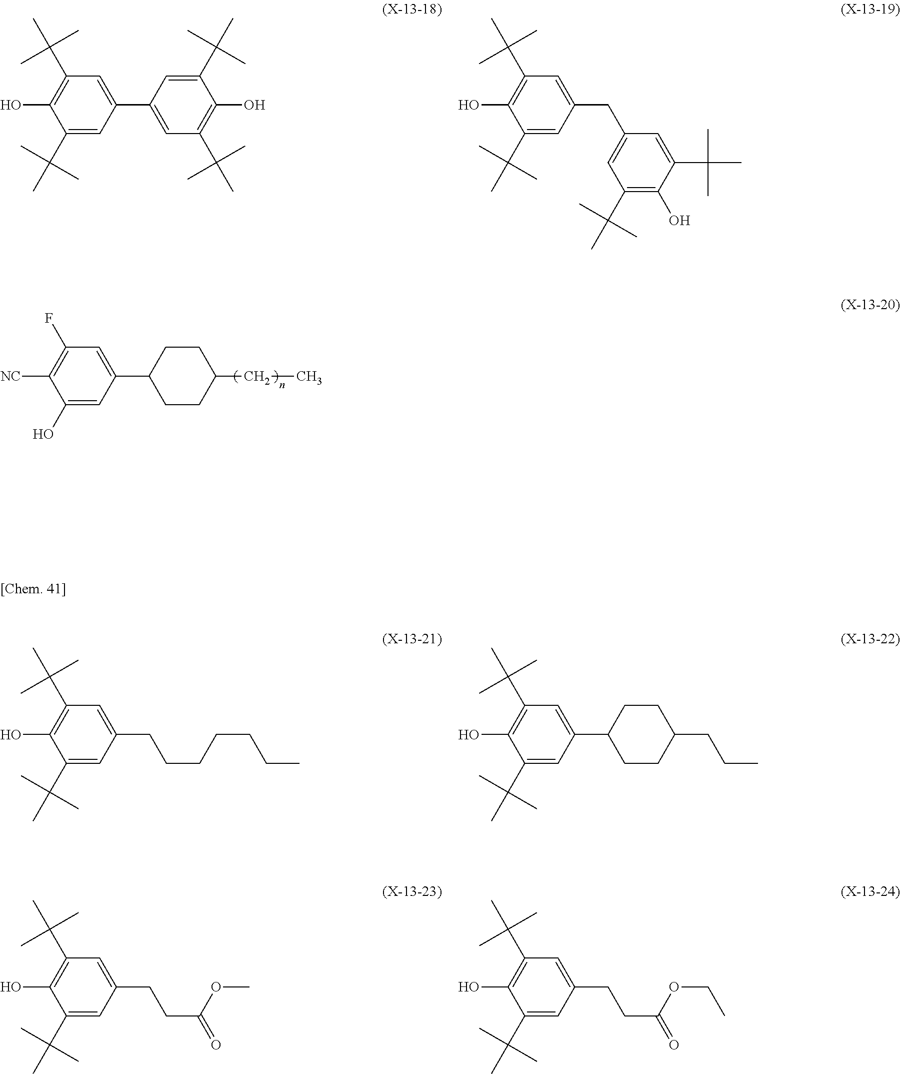

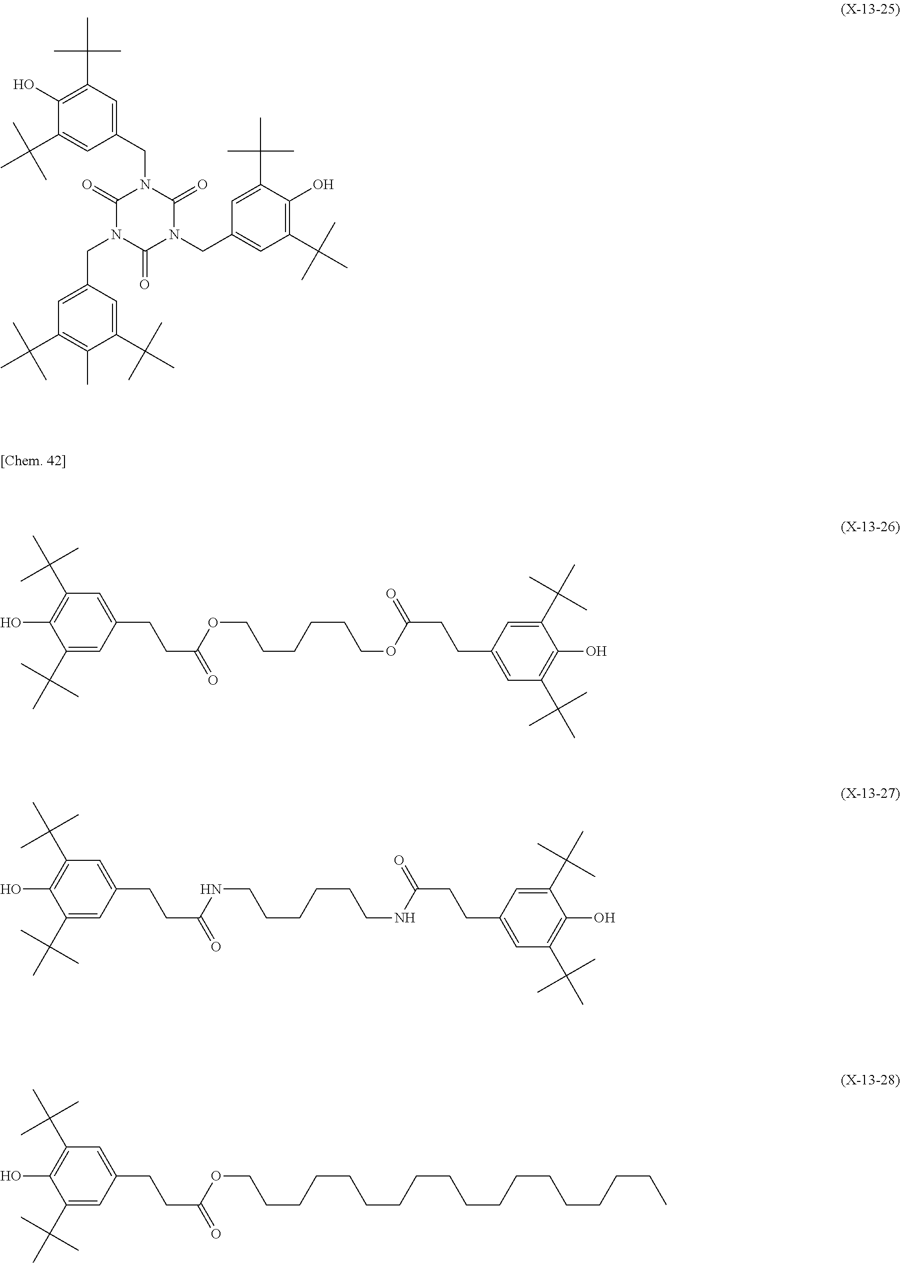 [SMILES: Cc1c(C(C)(C)C)cc(Cn2c(=O)n(Cc3cc(C(C)(C)C)c(O)c(C(C)(C)C)c3)c(=O)n(Cc3cc(C(C)(C)C)c(O)c(C(C)(C)C)c3)c2=O)cc1C(C)(C)C ; CC(C)(C)c1cc(CCC(=O)OCCCCCCOC(=O)CCc2cc(C(C)(C)C)c(O)c(C(C)(C)C)c2)cc(C(C)(C)C)c1O ; CC(C)(C)c1cc(CCC(=O)NCCCCCCNC(=O)CCc2cc(C(C)(C)C)c(O)c(C(C)(C)C)c2)cc(C(C)(C)C)c1O ; CCCCCCCCCCCCCCCCCCOC(=O)CCc1cc(C(C)(C)C)c(O)c(C(C)(C)C)c1]

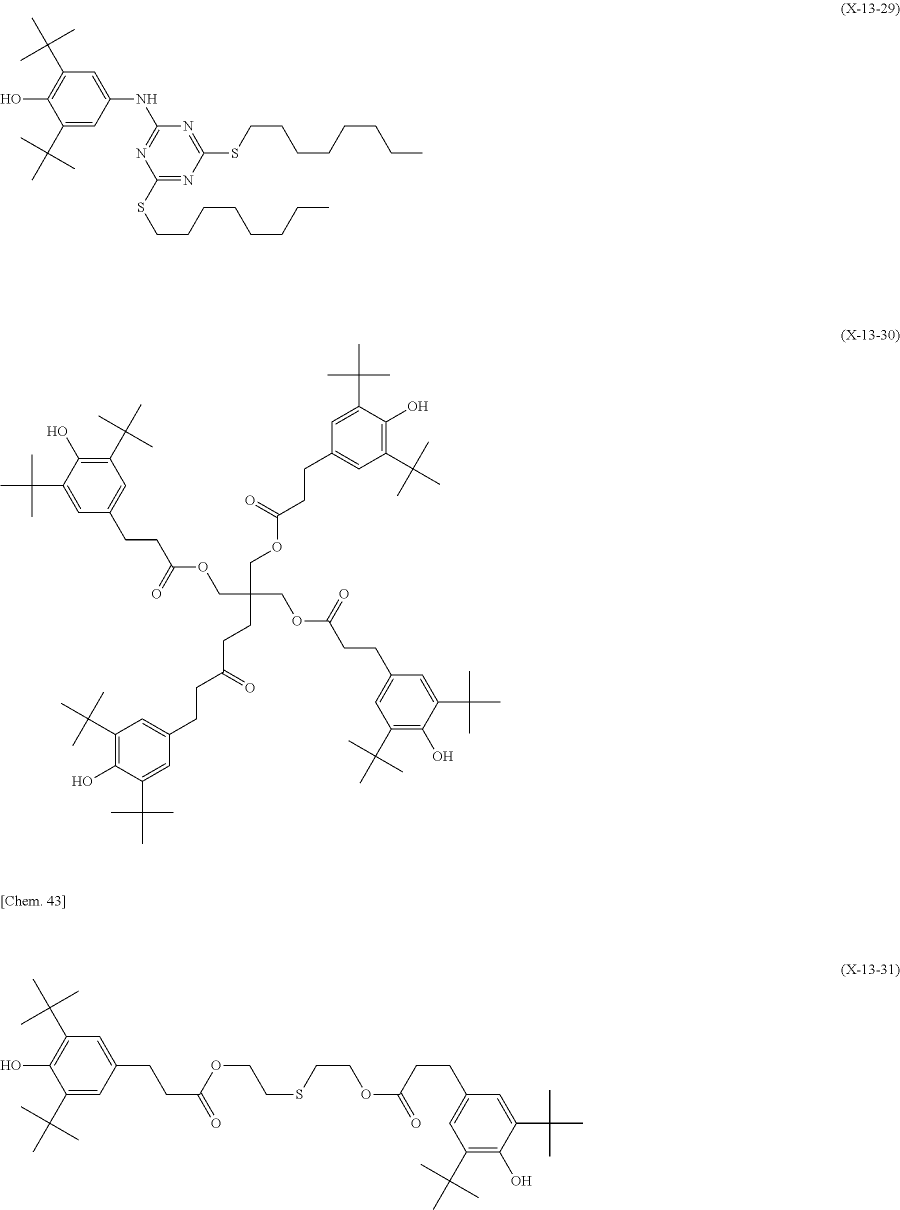

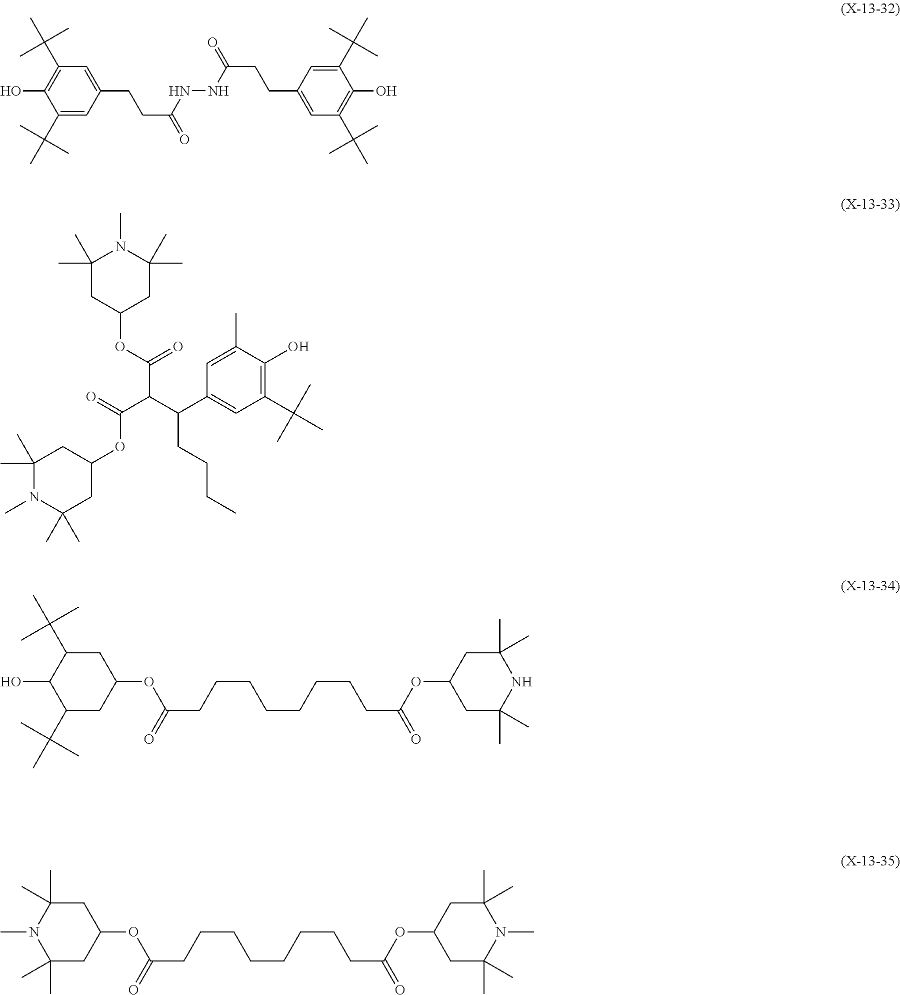

(in the formulae, n represents an integer of 0 to 20).

In addition, when the polymerizable liquid crystal composition containing the compound of the present invention is used for applications such as films, optical elements, functional pigments, medical products, cosmetics, coating agents, synthetic resins, etc., a metal, a metal complex, a dye, a pigment, a colorant, a fluorescent material, a phosphorescent material, a surfactant, a leveling agent, a thixotropic agent, a gelation agent, a polysaccharide, an ultraviolet absorber, an infrared absorber, an antioxidant, an ion exchange resin, a metal oxide such as titanium oxide or the like, etc. can be added according to purposes.

A polymer produced by polymerizing the polymerizable liquid crystal composition containing the compound of the present invention can be used for various applications. For example, a polymer produced by polymerizing, without alignment, the polymerizable liquid crystal composition containing the compound of the present invention can be used as a light scattering plate, a depolarizing plate, or a moire fringe prevention plate. Also, a polymer produced by polymerization after alignment has optical anisotropy and is thus useful. An optically anisotropic body can be produced by, for example, supporting the polymerizable liquid crystal composition containing the compound of the present invention on a substrate rubbed with a cloth or the like, a substrate having an organic thin film formed thereon, or a substrate having an alignment film formed by oblique vapor deposition of SiO₂ or holding the polymerizable liquid crystal composition between substrates and then polymerizing the polymerizable liquid crystal composition.

Examples of a method for supporting the polymerizable liquid crystal composition on the substrate include spin coating, die coating, extrusion coating, roll coating, wire bar coating, gravure coating, spray coating, dipping, printing, and the like. During coating, an organic solvent may be added to the polymerizable liquid crystal composition. Usable examples of the organic solvent include a hydrocarbon solvent, a halogenated hydrocarbon solvent, an ether solvent, an alcohol solvent, a ketone solvent, an ester solvent, an aprotic solvent, and the like. Examples of the hydrocarbon solvent include toluene and hexane; examples of the halogenated hydrocarbon solvent include methylene chloride; examples of the ether solvent include tetrahydrofuran, acetoxy-2-ethoxyethane propylene glycol monomethyl ether acetate; examples of the alcohol solvent include methanol, ethanol, and isopropyl alcohol; examples of the ketone solvent include acetone, methyl ethyl ketone, cyclohexanone, γ-butyrolactone, N-methyl pyrrolidinone; examples of the ester solvent include ethyl acetate and cellosolve; and examples of the aprotic solvent include dimethyl formamide and acetonitrile. These may be used alone or in combination, and the organic solvent may be properly selected in consideration of vapor pressure and solubility of the polymerizable liquid crystal composition. In addition, natural drying, heat drying, reduced-pressure drying, or reduced-pressure heat drying can be used as a method for evaporating the organic solvent added. In order to further improve coatability of a polymerizable liquid crystal material, it is effective to provide an intermediate layer such as a polyimide thin film or the like on the substrate or to add a leveling agent to the polymerizable liquid crystal material. The method of providing an intermediate layer such as a polyimide thin film or the like on the substrate is effective for improving adhesion between the substrate and the polymer produced by polymerizing the polymerizable liquid crystal material.

Besides the alignment treatment described above, fluid alignment of the liquid crystal material or an electric field or magnetic field can also be used. These alignment methods may be used alone or used in combination. Further, an optical alignment method can be used as the alignment treatment method in place of rubbing. Besides a plate shape, the substrate may have a shape having a curved surface as a constituent part. Both an organic material and an inorganic material can be used as the material constituting the substrate. Examples of the organic material used as the material of the substrate include polyethylene terephthalate, polycarbonate, polyimide, polyamide, polymethyl methacrylate, polystyrene, polyvinyl chloride, polytetrafluoroethylene, polychlorotrifluoroethylene, polyarylate, polysulfone, triacetyl cellulose, cellulose, polyether ether ketone, and the like, and examples of the inorganic material include silicon, glass, calcite, and the like.

In polymerizing the polymerizable liquid crystal composition containing the compound of the present invention, a method of polymerization by irradiation with active energy rays such as ultraviolet light or electron beams, or the like is preferred because the polymerization is desired to rapidly proceed. When ultraviolet light is used, either a polarized light source or an unpolarized light source may be used. Also, when the liquid crystal composition is polymerized in a state of being held between two substrates, at least the substrate on the irradiation surface side is required to have proper transparency to active energy rays. Another method may also be used, which includes, after polymerization of only a specified portion using a mask during irradiation, changing the alignment state of an unpolymerized portion by changing a condition such as an electric field, a magnetic field, a temperature, or the like and further performing polymerization by irradiation with active energy rays. The temperature during irradiation is preferably within a temperature range in which the liquid crystal state of the polymerizable liquid crystal composition of the present invention is maintained. In particular, when the optically anisotropic body is produced by photopolymerization, the polymerization is preferably performed at a temperature as close to room temperature as possible, that is, typically a temperature of 25° C., in view of avoiding induction of undesired therraopolymerization. The intensity of active energy rays is preferably 0.1 mW/cm² to 2 W/cm². The intensity of 0.1 mW/cm² or less requires much time for completing photopolymerization and thus causes deterioration in productivity, while the intensity of 2 W/cm² or more causes the danger of degrading the polymerizable liquid crystal compound or the polymerizable liquid crystal composition.

The optically anisotropic body produced by polymerization can be subjected to heat treatment for the purpose of decreasing changes in the initial characteristics and attempting to exhibit stable characteristics. The temperature of heat treatment is preferably within a range of 50° C. to 250° C., and time of heat treatment is preferably within 30 seconds to 12 hours.

The optically anisotropic body produced by the method described above may be used as a single body separated from the substrate or used without being separated from the substrate. Also, the resultant optically anisotropic body may be used by stacking or by laminating on another substrate.

### EXAMPLES

The present invention is further described below by giving examples, but the present invention is not limited to these examples. In addition, in compositions of examples and comparative examples below, “%” represents “% by mass”. When a substance unstable for oxygen and/or moisture is handled in each of the steps, an operation is preferably performed in inert gas such as nitrogen gas, argon gas, or the like. Usual post-treatment is an operation for obtaining an intended compound from a reaction solution and represents an operation generally performed by a person skilled in the art, such as separation, extraction, neutralization, washing, drying, concentration, or the like.

### Example 1

### Production of Compound Represented by Formula (I-1)

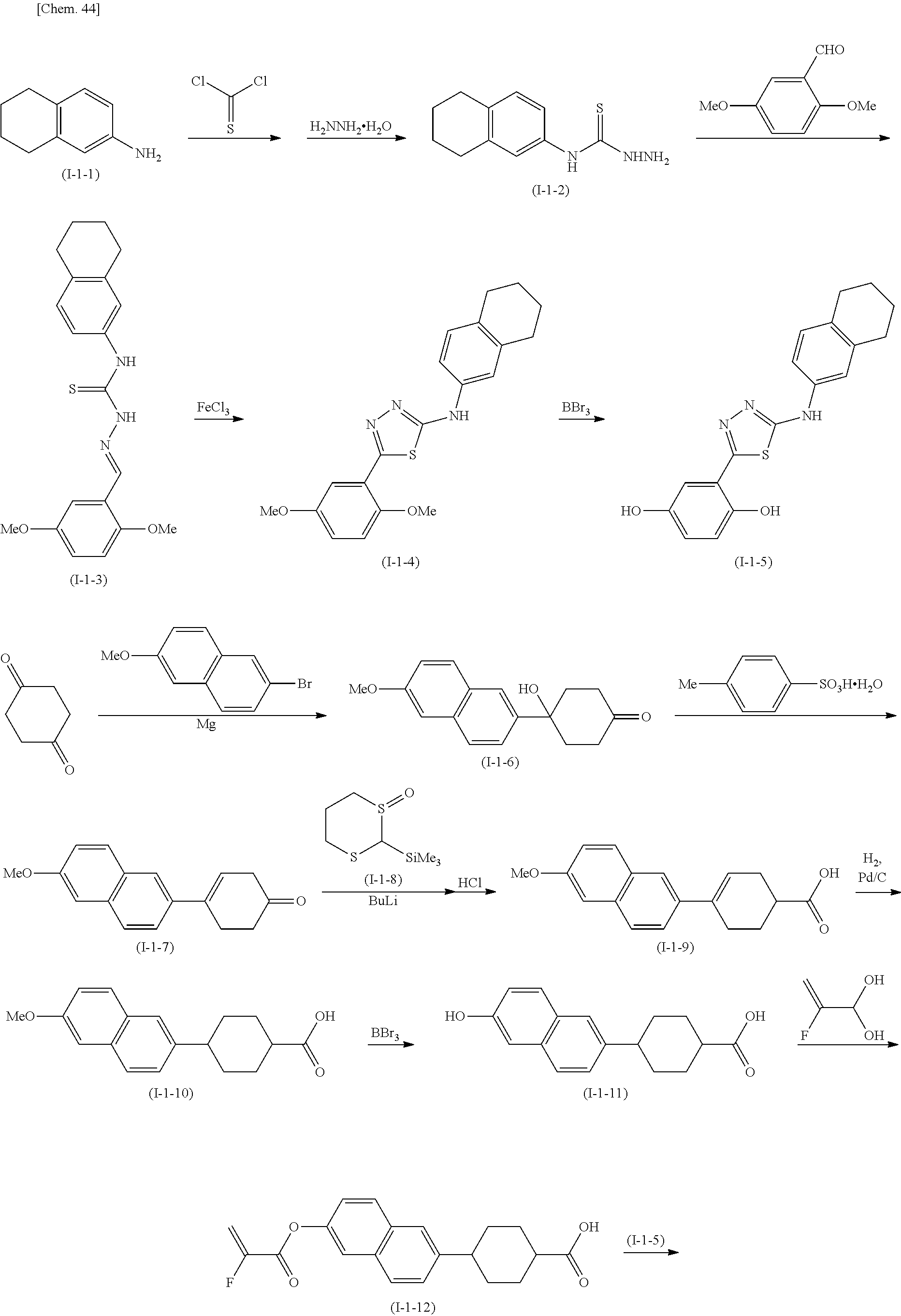

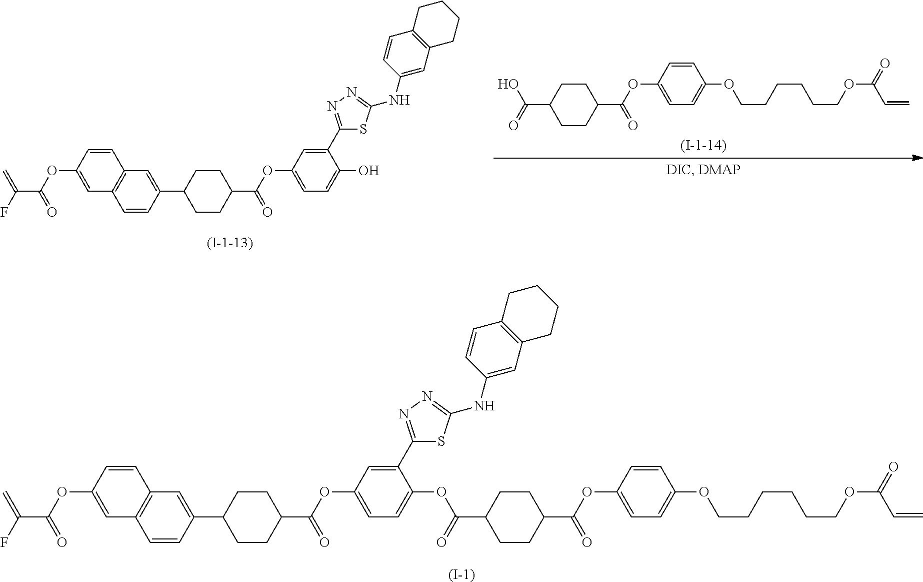

A compound represented by the formula (I-1-1) was produced by the method described in Journal of Medicinal Chemistry, 2009, Vol. 52, No. 9, pp. 2989-3000. The compound represented by the formula (I-1-1), triethylamine, and ethyl acetate were added to a reactor. Then, an ethyl acetate solution of thiophosgene was added dropwise and stirred under ice cooling. The reaction solution was poured into water, and purification was performed by column chromatography after usual post-treatment. The resultant compound was dissolved in 2-propanol, and the resultant solution was added dropwise to a reactor to which hydrazine monohydrate and 2-propanol had been added, followed by stirring. The precipitate was filtered off and dried to produce a compound represented by the formula (I-1-2).

The compound represented by the formula (I-1-2), 2,5-dimethoxybenzaldehyde, and ethanol were added to a reactor. The precipitate was filtered off and dried to produce a compound represented by the formula (I-1-3).

The compound represented by the formula (I-1-3), tetrahydrofuran, ethanol, and water were added to a reactor. Then, iron(III) chloride was added and stirred. The precipitate was filtered off and dried to produce a compound represented by the formula (I-1-4).

The compound represented by the formula (I-1-4) and dichloromethane were added to a reactor. Then, the resultant mixture was cooled to −78° C. and boron tribromide was added and stirred. The reaction solution was poured into water, and purification was performed by column chromatography and recrystallization after usual post-treatment, thereby producing a compound represented by the formula (I-1-5).

Magnesium and tetrahydrofuran were added to a reactor. Then, a tetrahydrofuran solution of 6-bromo-2-methoxynaphthalene was added to prepare a Grignard reagent. Then, a tetrahydrofuran solution of 1,4-dichlorohexanedione was added dropwise and stirred. Then, hydrochloric acid was added dropwise, and purification was performed by column chromatography after usual post-treatment, thereby producing a compound represented by the formula (I-1-6).

The compound represented by the formula (I-1-6), p-toluenesulfonic acid monohydrate, and toluene were added to a reactor, and heated under reflux while water was removed. Then, purification was performed by column chromatography after usual post-treatment, thereby producing a compound represented by the formula (I-1-7).

A compound represented by the formula (I-1-8) was produced by the method described in Synthesis, 2010, No. 15, pp. 2616-2620. The compound represented by the formula (I-1-8) and tetrahydrofuran were added to a reactor. Then, a hexane solution of butyllithium was added dropwise and stirred under cooling to −78° C. A tetrahydrofuran solution of the compound represented by the formula (I-1-7) was added dropwise and then the resultant mixture was stirred at room temperature. The reaction solution was poured into an aqueous ammonium chloride solution, and purification was performed by column chromatography after usual post-treatment. The resultant compound, acetonitrile, and 6M hydrochloric acid were added to a reactor and heated under stirring. The reaction solution was poured into water, and purification was performed by column chromatography after usual post-treatment, thereby producing a compound represented by the formula (I-1-9).

The compound represented by the formula (I-1-9), 5% palladium carbon, and ethanol were added to a reactor and stirred under a hydrogen pressure of 0.5 MPa. The palladium carbon was filtered off, and the solvent was distilled off to produce a compound represented by the formula (I-1-10).

The compound represented by the formula (I-1-10) and dichloromethane were added to a reactor. Then, the resultant mixture was cooled to −78° C. and boron tribromide was added and stirred. The reaction solution was poured into water, and purification was performed by column chromatography and recrystallization after usual post-treatment, thereby producing a compound represented by the formula (I-1-11).

The compound represented by the formula (I-1-11), 2-fluoroacrylic acid, N,N-dimethylaminopyridine, and dichloromethane were added to a reactor. Then, diisopropyl carbodiimide was added dropwise and stirred under ice cooling. Then, purification was performed by column chromatography after usual post-treatment, thereby producing a compound represented by the formula (I-1-12).

The compound represented by the formula (I-1-12), the compound represented by the formula (I-1-5), N,N-dimethylaminopyridine, and dichloromethane were added to a reactor. Then, diisopropyl carbodiimide was added dropwise and stirred under ice cooling. Then, purification was performed by column chromatography after usual post-treatment, thereby producing a compound represented by the formula (I-1-13).

A compound represented by the formula (I-1-14) was produced by the method described in Publication No. WO2009-116657A1. The compound represented by the formula (I-1-13), the compound represented by the formula (I-1-14), N,N-dimethylaminopyridine, and dichloromethane were added to a reactor. Then, diisopropyl carbodiimide was added dropwise and stirred under ice cooling. Then, purification was performed by column chromatography and recrystallization after usual post-treatment, thereby producing a compound represented by the formula (I-1).

MS (m/z): 1064 [M⁺+1]

### Example 2

### Production of Compound Represented by Formula (I-2)

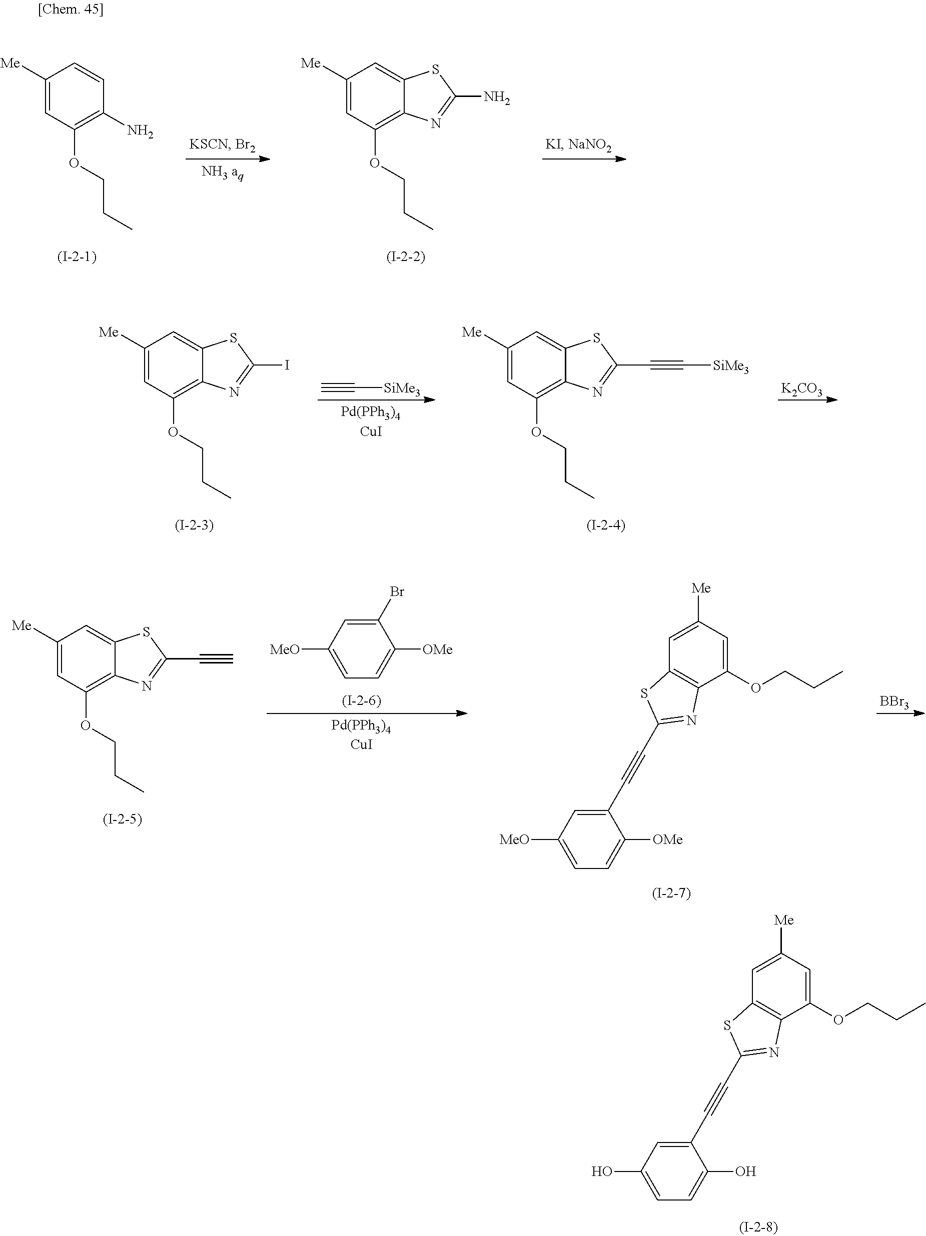

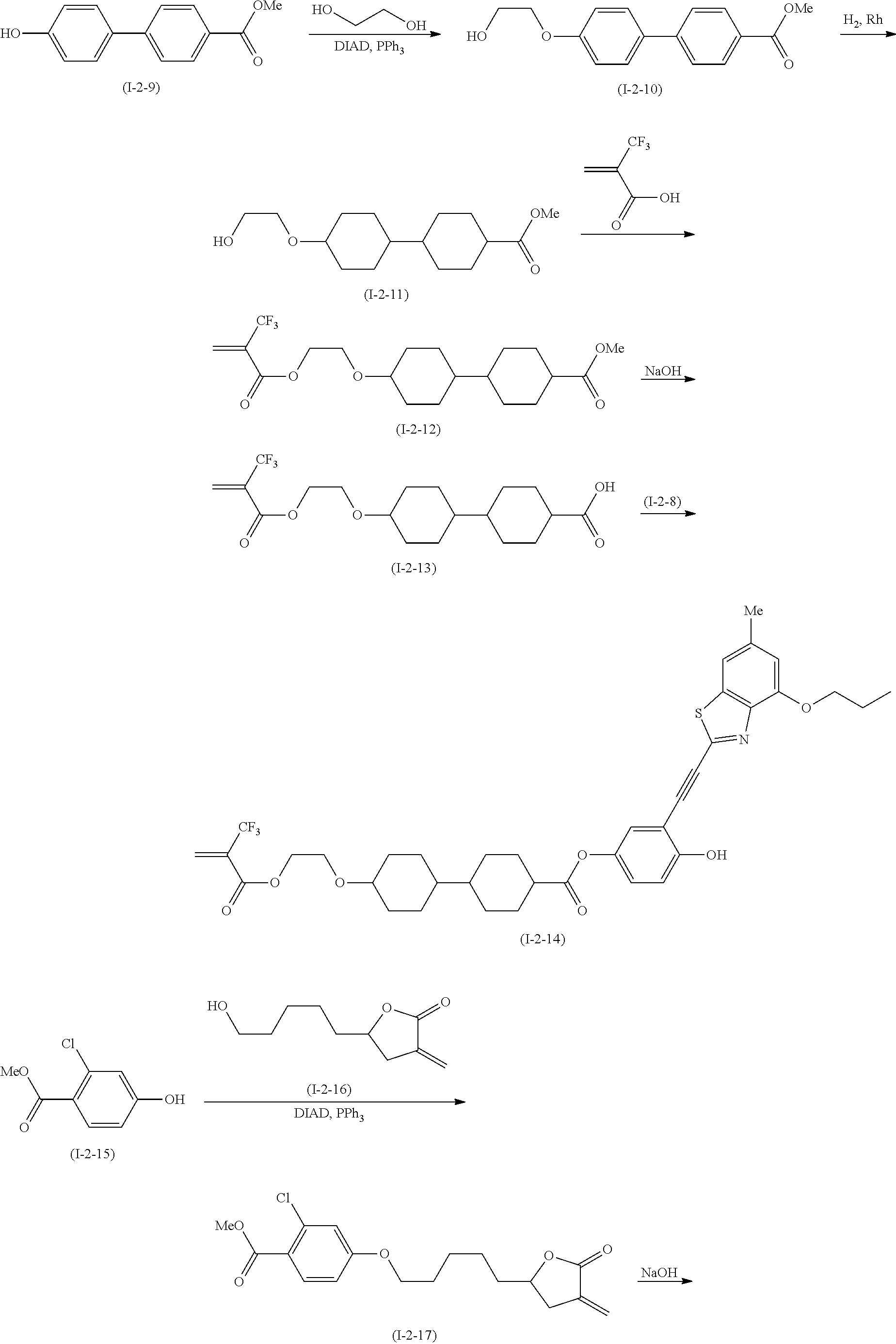

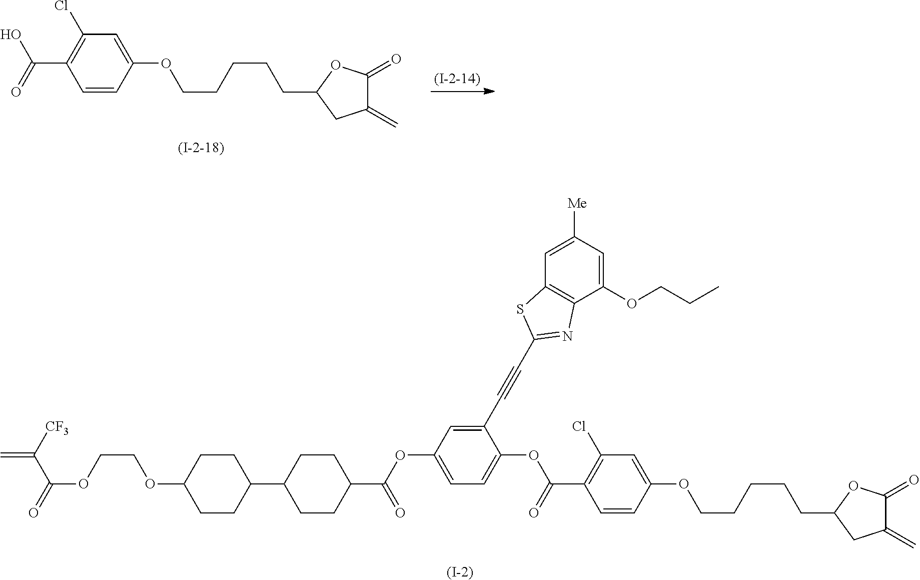 [SMILES: C=C1CC(CCCCCOc2ccc(C(=O)O)c(Cl)c2)OC1=O ; C=C1CC(CCCCCOc2ccc(C(=O)Oc3ccc(OC(=O)C4CCC(C5CCC(OCCOC(=O)C(=C)C(F)(F)F)CC5)CC4)cc3C#Cc3nc4c(OCCC)cc(C)cc4s3)c(Cl)c2)OC1=O ; "(I-2)"]

A compound represented by the formula (I-2-1), potassium thiocyanate, and acetic acid were added to a reactor. Then, bromine was added dropwise and stirred under ice cooling. Then, the precipitate was filtered off and dried. The resultant solid was dissolved in hot water, and an aqueous ammonia solution was added to the resultant-solution, followed by stirring. The solid was filtered off and purification was performed by column chromatography and recrystallization to produce a compound represented by the formula (I-2-2).

The compound represented by the formula (I-2-2), p-toluenesulfonic acid monohydrate, and acetonitrile were added to a reactor. Then, an aqueous sodium nitride solution and an aqueous potassium iodide solution were added dropwise under ice cooling and stirred at room temperature. Then, purification was performed by column chromatography after usual post-treatment, thereby producing a compound represented by the formula (I-2-3).

The compound represented by the formula (I-2-3), trimethylsilyl acetylene, copper(I) iodide, tetrakis(triphenylphosphine) palladium(0), triethylamine, and N,N-dimethylformamide were added to a reactor and heated and stirred. Then, purification was performed by column chromatography after usual post-treatment, thereby producing a compound represented by the formula (I-2-4).

The compound represented by the formula (I-2-4), potassium carbonate, and methanol were added to a reactor and stirred. Then, purification was performed by column chromatography after usual post-treatment, thereby producing a compound represented by the formula (I-2-5).

The compound represented by the formula (I-2-5), a compound represented by the formula (I-2-6), copper(I) iodide, tetrakis(triphenylphosphine) palladium(0), triethylamine, and N,N-dimethylformamide were added to a reactor and heated and stirred. Then, purification was performed by column chromatography after usual post-treatment, thereby producing a compound represented by the formula (I-2-7).

The compound represented by the formula (I-2-7) and dichloromethane were added to a reactor. Then, boron tribromide was added dropwise and stirred at −78° C. The reaction solution was poured into water, and purification was performed by column chromatography after usual post-treatment, thereby producing a compound represented by the formula (I-2-8).

A compound represented by the formula (I-2-9), ethylene glycol, triphenylphosphine, and tetrahydrofuran were added to a reactor. Then, diisopropyl azodicarboxylate was added dropwise and stirred. Then, purification was performed by column chromatography after usual post-treatment, thereby producing a compound represented by the formula (I-2-10).

The compound represented by the formula (I-2-10), rhodium, and diisopropyl alcohol were added to a reactor and heated and stirred under a hydrogen pressure of 5 atm. The catalyst was removed, and then purification was performed by column chromatography, thereby producing a compound represented by the formula (I-2-11).

The compound represented by the formula (I-2-11), 2-(trifluoromethyl) acrylic acid, N,N-dimethylaminopyridine, and dichloromethane were added to a reactor. Then, diisopropyl carbodiimide was added dropwise and stirred. Then, purification was performed by column chromatography after usual post-treatment, thereby producing a compound represented by the formula (I-2-12).

The compound represented by the formula (I-2-12), methanol, and an aqueous sodium hydroxide solution were added to a reactor. Then, the resultant mixture was neutralized with hydrochloric acid, and purification was performed by column chromatography after usual post-treatment, thereby producing a compound represented by the formula (I-2-13).

The compound represented by the formula (I-2-13), the compound represented by the formula (I-2-8), N,N-dimethylaminopyridine, and dichloromethane were added to a reactor. Then, diisopropyl carbodiimide was added dropwise and stirred. Then, purification was performed by column chromatography after usual post-treatment, thereby producing a compound represented by the formula (I-2-14).

A compound represented by the formula (I-2-16) was produced by the method described in Synthesis, 2001, No. 10, pp. 1519-1522. The compound represented by the formula (I-2-15), the compound represented by the formula (I-2-16), triphenylphosphine, and tetrahydrofuran were added to a reactor. Then, diisopropyl azodicarboxylate was added dropwise and stirred. Then, purification was performed by column chromatography after usual post-treatment, thereby producing a compound represented by the formula (I-2-17).

The compound represented by the formula (I-2-17), methanol, and an aqueous sodium hydroxide solution were added to a reactor. Then, the resultant mixture was neutralized with hydrochloric acid, and purification was performed by column chromatography after usual post-treatment, thereby producing a compound represented by the formula (I-2-18).

The compound represented by the formula (I-2-18), the compound represented by the formula (I-2-14), N,N-dimethylaminopyridine, and dichloromethane were added to a reactor. Then, diisopropyl carbodiimide was added dropwise and stirred. Then, purification was performed by column chromatography and recrystallization after usual post-treatment, thereby producing a compound represented by the formula (I-2).

MS (m/z): 1034 [M⁺+1]

### Example 3

### Production of Compound Represented by Formula (I-3)

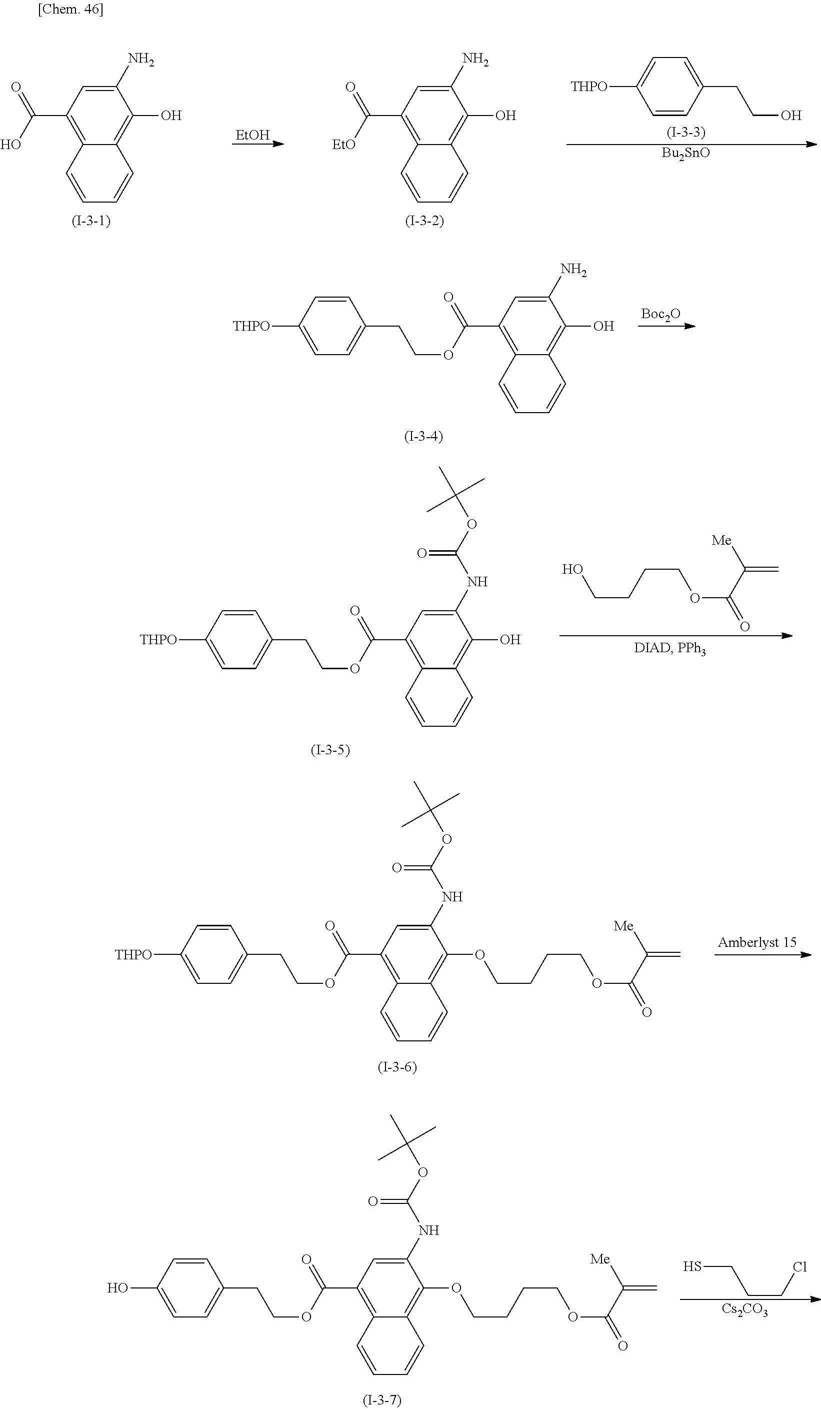

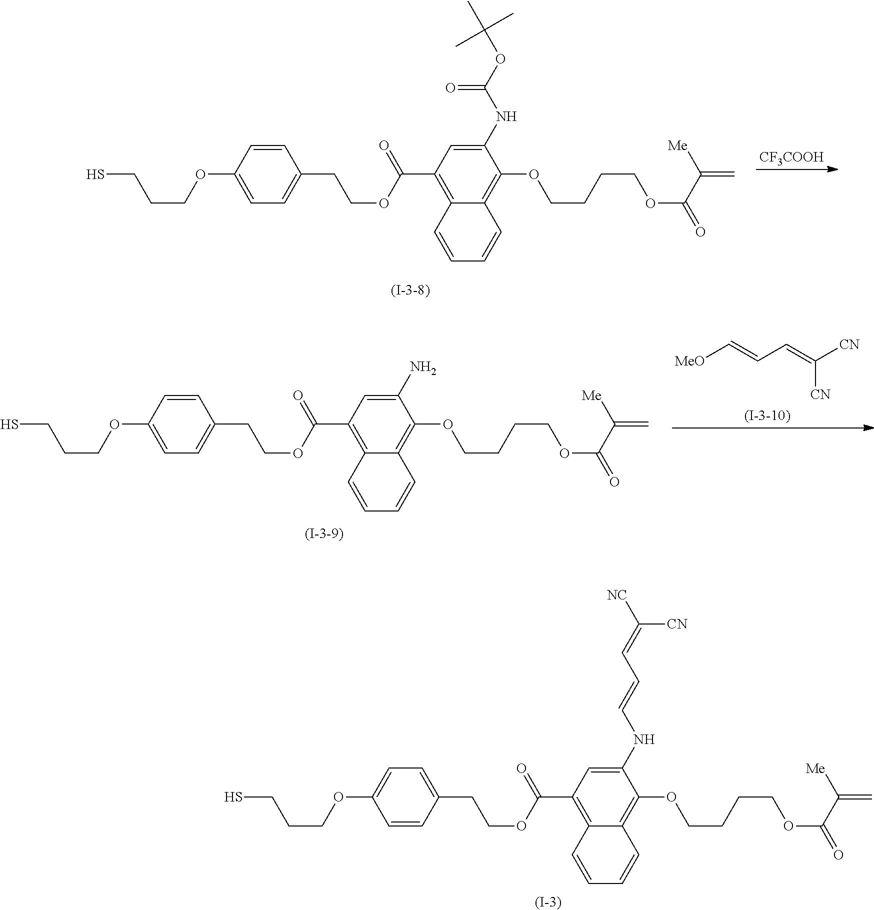 [SMILES: C=C(C)C(=O)OCCCCOc1c(NC(=O)OC(C)(C)C)cc(C(=O)OCCc2ccc(OCCCS)cc2)c2ccccc12 ; "CF3COOH" ; C=C(C)C(=O)OCCCCOc1c(N)cc(C(=O)OCCc2ccc(OCCCS)cc2)c2ccccc12 ; CO/C=C/C=C(C#N)C#N ; C=C(C)C(=O)OCCCCOc1c(N/C=C/C=C(C#N)C#N)cc(C(=O)OCCc2ccc(OCCCS)cc2)c2ccccc12 ; "(I-3)"]

A compound represented by the formula (I-3-1), concentrated sulfuric acid, and ethanol were added to a reactor and heated under reflux. Then, the resultant mixture was diluted with ethyl acetate, and purification was performed by column chromatography after usual post-treatment, thereby producing a compound represented by the formula (I-3-2).

A compound represented by the formula (I-3-3) was produced by the method described in Tetrahedron Letters, 2010, Vol. 51, No. 17, pp. 2323-2325. The compound represented by the formula (I-3-2), the compound represented by the formula (I-3-3), dibutyltin oxide, and toluene were added to a reactor and heated under reflux while the solvent was replaced. Then, purification was performed by column chromatography after usual post-treatment to produce a compound represented by the formula (I-3-4).

The compound represented by the formula (I-3-4), di-tert-butyl dicarbonate, and tetrahydrofuran were added to a reactor and heated under reflux. The solvent was distilled off, and then purification was performed by column chromatography, thereby producing a compound represented by the formula (I-3-5).

The compound represented by the formula (I-3-5), 4-hydroxybutyl methacrylate, triphenylphosphine, and tetrahydrofuran were added to a reactor. Then, diisopropyl azodicarboxylate was added dropwise under ice cooling. Then, purification was performed by column chromatography after usual post-treatment, thereby producing a compound represented by the formula (I-3-6).

The compound represented by the formula (I-3-6), methanol, and Amberlyst 15 were added to a reactor and heated under reflux. The solution was filtered, then the solvent was distilled off, and purification was performed by column chromatography, thereby producing a compound represented by the formula (I-3-7).

The compound represented by the formula (I-3-7), 3-chloro-1-propanethiol, cesium carbonate, and dimethylsulfoxide were added to a reactor and heated and stirred. The resultant mixture was diluted with dichloromethane, and purification was performed by column chromatography after usual post-treatment, thereby producing a compound represented by the formula (I-3-8).

The compound represented by the formula (I-3-8) and dichloromethane were added to a reactor. Then, trifiuoroacetic acid was added dropwise and stirred under ice cooling. Then, purification was performed by column chromatography after usual post-treatment, thereby producing a compound represented by the formula (I-3-9).

The compound represented by the formula (I-3-9), triethylamine, and ethyl acetate were added to a reactor. Then, a compound represented by the formula (I-3-10) was added and heated under reflux. Then, purification was performed by column chromatography to produce a compound represented by the formula (I-3).

MS (m/z): 640 [M⁺+1]

### Example 4

### Production of Compound Represented by Formula (I-4)

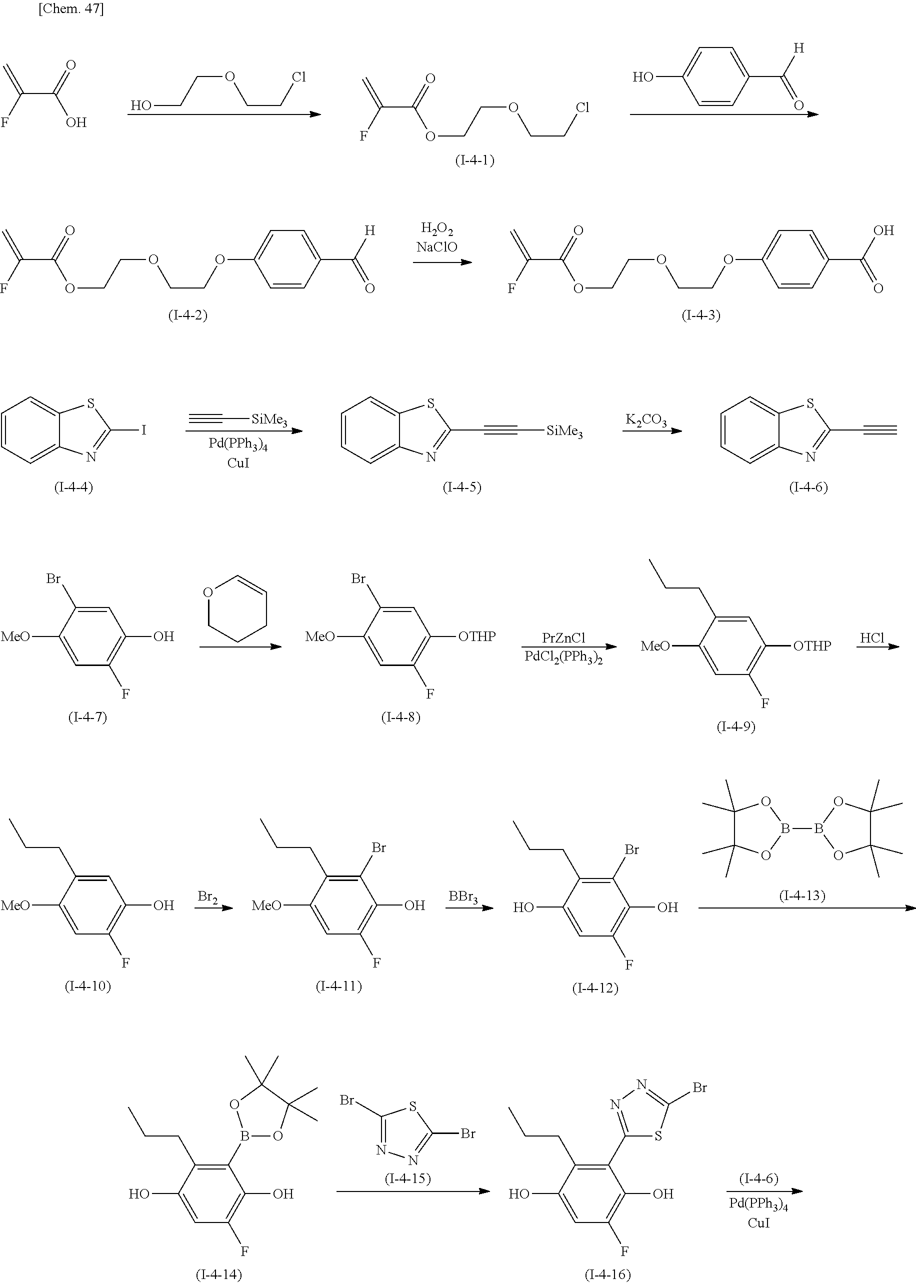

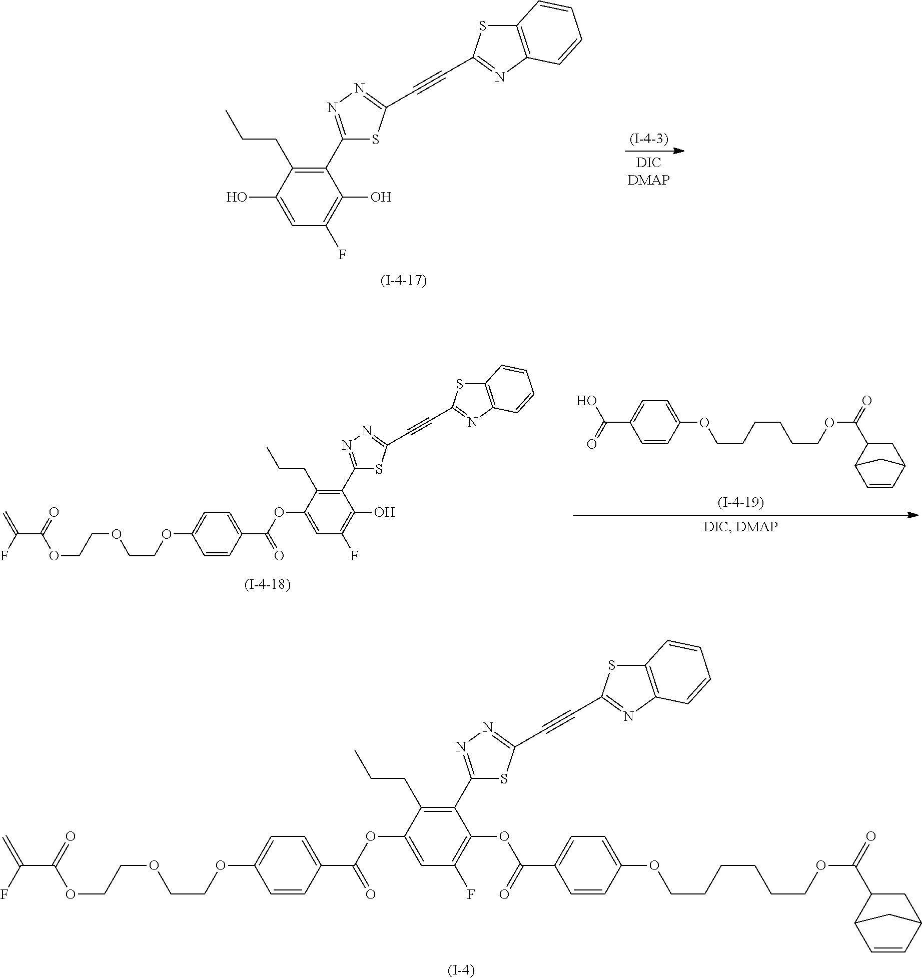 [SMILES: CCCc1c(O)cc(F)c(O)c1-c1nnc(C#Cc2nc3ccccc3s2)s1 ; C=C(F)C(=O)OCCOCCOc1ccc(C(=O)Oc2cc(F)c(O)c(-c3nnc(C#Cc4nc5ccccc5s4)s3)c2CCC)cc1 ; O=C(O)c1ccc(OCCCCCCOC(=O)C2CC3C=CC2C3)cc1 ; C=C(F)C(=O)OCCOCCOc1ccc(C(=O)Oc2cc(F)c(OC(=O)c3ccc(OCCCCCCOC(=O)C4CC5C=CC4C5)cc3)c(-c3nnc(C#Cc4nc5ccccc5s4)s3)c2CCC)cc1 ; "(I-4)"]

First, 2-fluoroacrylic acid, ethylene glycol mono-2-chloroethyl ether, N,N-dimethylaminopyridine, and dichloromethane were added to a reactor. Then, diisopropyl carbodiimide was added dropwise and stirred under ice cooling. Then, purification was performed by column chromatography after usual post-treatment, thereby producing a compound represented by the formula (I-4-1).

The compound represented by the formula (I-4-1), 4-hydroxybenzaldehyde, cesium carbonate, and dimethylsulfoxide were added to a reactor and heated and stirred. Then, purification was performed by column chromatography after usual post-treatment to produce a compound represented by the formula (I-4-2).

The compound represented by the formula (I-4-2), sodium dihydrogen phosphate dihydrate, methanol, water, and an aqueous hydrogen peroxide solution were added to a reactor. Then, an aqueous sodium chlorite solution was added dropwise and heated and stirred. The resultant mixture was cooled by adding water, and the precipitate was filtered off and dried to produce a compound represented by the formula (I-4-4). The compound represented by the formula (I-4-4), trimethylsilyl acetylene, copper(I) iodide, triethylamine, and N,N-dimethylformamide were added to a reactor and heated and stirred. Then, purification was performed by column chromatography after usual post-treatment to produce a compound represented by the formula (I-4-5).

The compound represented by the formula (I-4-5), methanol, and potassium carbonate were added to a reactor and stirred. Then, purification was performed by column chromatography after usual post-treatment to produce a compound represented by the formula (I-4-6).

The compound represented by the formula (I-4-7), pyridinium p-toluenesulfonate, and dichloromethane were added to a reactor. Then, 3,4-dihydro-2H-pyrane was added dropwise and stirred. Then, purification was performed by column chromatography after usual post-treatment to produce a compound represented by the formula (I-4-8).

First, sine chloride and tetrahydrofuran were added to a reactor. Then, a tetrahydrofuran solution of propylmagnesium bromide was added dropwise and stirred. The resultant reaction solution was added dropwise to a reactor in which a compound represented by the formula (I-4-3), tetrahydrofuran, and bis(triphenylphosphine)palladium(II) dichloride had been mixed. The resultant mixture was heated and stirred, and then purification was performed by column chromatography after usual post-treatment to produce a compound represented by the formula (I-4-9).

The compound represented by the formula (I-4-9), methanol, and concentrated hydrochloric acid were added to a reactor. Then, purification was performed by column chromatography after usual post-treatment to produce a compound represented by the formula (I-4-10).

The compound represented by the formula (I-4-10) and dichloromethane were added to a reactor. The resultant mixture was cooled, and bromine was added dropwise and stirred. The reaction solution was poured into water, and then purification was performed by column chromatography after usual post-treatment to produce a compound represented by the formula (I-4-11).

The compound represented by the formula (I-4-11) and dichloromethane were added to a reactor. The resultant mixture was cooled to −78° C., and boron tribromide was added dropwise and stirred. The reaction solution was poured into water, and then purification was performed by column chromatography after usual post-treatment to produce a compound represented by the formula (I-4-12).

The compound represented by the formula (I-4-12), a compound represented by the formula (I-4-13), potassium acetate, bis(triphenylphosphine) palladium(II) dichloride, and dimethylsulfoxide were added to a reactor and heated and stirred. Then, purification was performed by column chromatography after usual post-treatment to produce a compound represented by the formula (I-4-14).

The compound represented by the formula (I-4-14), a compound represented by the formula (I-4-15), potassium carbonate, tetrakis(triphenylphosphine)palladium(0), ethanol, and water were added to a reactor and heated under reflux. Then, purification was performed by column chromatography after usual post-treatment to produce a compound represented by the formula (I-4-16).

The compound represented by the formula (I-4-16), the compound represented by the formula (I-4-6), copper(I) iodide, triethylamine, N,N-dimethylformamide, and tetrakis(triphenylphosphine)palladium(0) were added to a reactor and heated and stirred. Then, purification was performed by column chromatography after usual post-treatment to produce a compound represented by the formula (I-4-17).

The compound represented by the formula (I-4-17), the compound represented by the formula (I-4-3), N,N-dimethylaminopyridine, and dichloromethane were added to a reactor. Then, diisopropyl carbodiimide was added dropwise and stirred under ice cooling. Then, purification was performed by column chromatography after usual post-treatment to produce a compound represented by the formula (I-4-18).

A compound represented by the formula (I-4-19) was produced by the method described in Publication No. WO993770A1. The compound represented by the formula (I-4-18), the compound represented by the formula (I-4-19), N,N-dimethylaminopyridine, and dichloromethane were added to a reactor. Then, diisopropyl carbodiimide was added dropwise and stirred. Then, purification was performed by column chromatography and recrystallization after usual post-treatment to produce a compound represented by the formula (I-4).

MS (m/z): 1032 [M⁺+1]

### Example 5

### Production of Compound Represented by Formula (I-5)

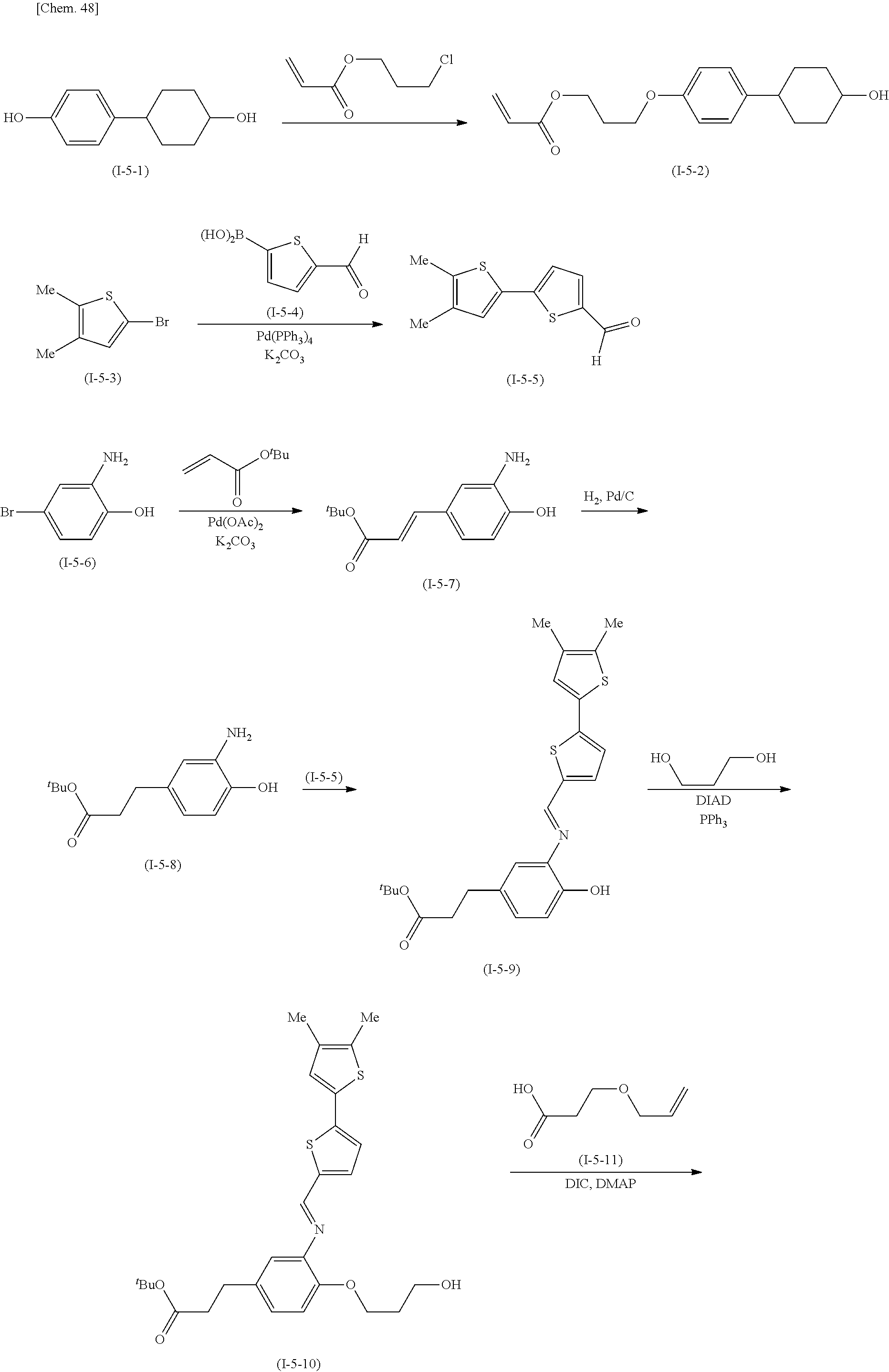

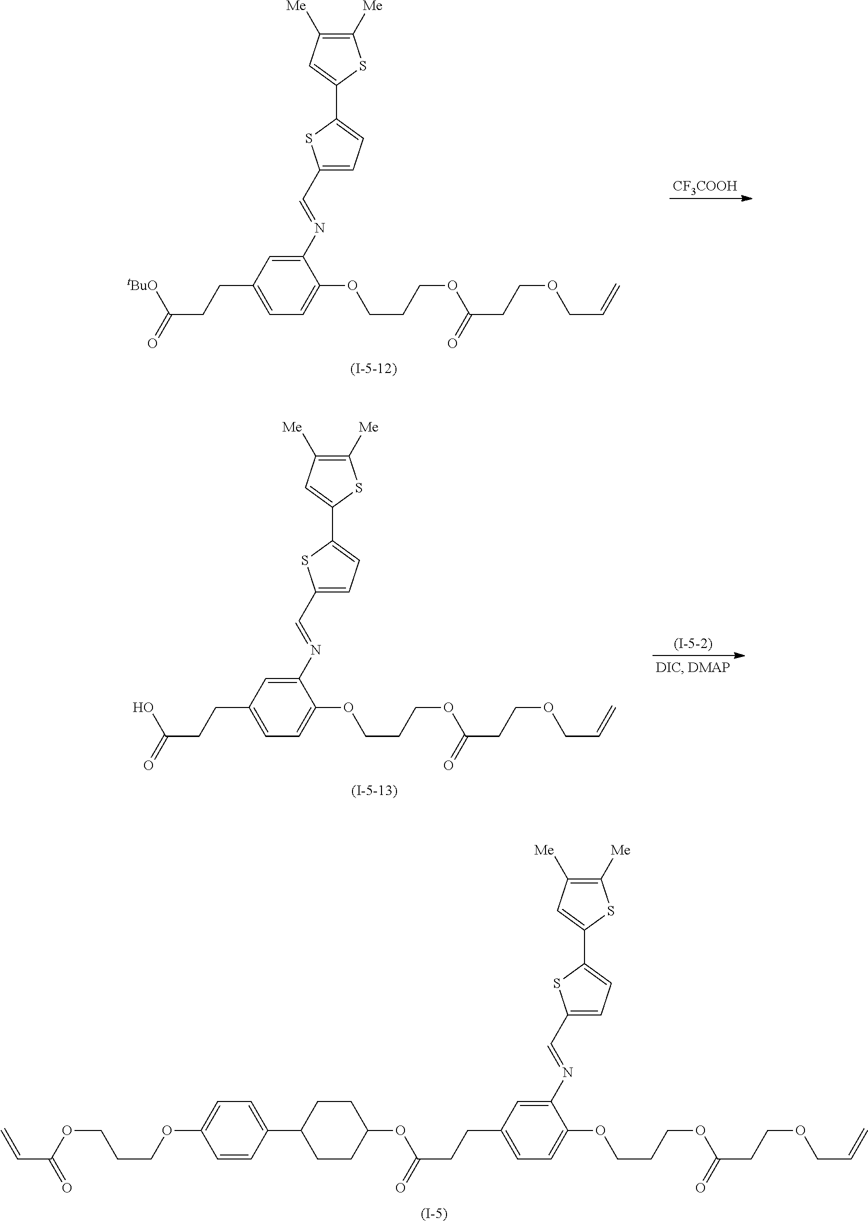

A compound represented by the formula (I-5-1), 3-chloropropyl acrylate, cesium carbonate, and dimethylsuifoxide were added to a reactor and heated and stirred. Then, purification was performed by column chromatography after usual post-treatment to produce a compound represented by the formula (I-5-2).

A compound represented by the formula (I-5-3), a compound represented by the formula (I-5-4), potassium carbonate, ethanol, and tetrakis(triphenylphosphine) palladium(0) were added to a reactor and heated under reflux. Then, purification was performed by column chromatography after usual post-treatment to produce a compound represented by the formula (I-5-5).

A compound represented by the formula (I-5-6), tert-butyl acrylate, potassium carbonate, and N,N-dimethylformamide, palladium(II) acetate were added to a reactor and heated and stirred. Then, purification was performed by column chromatography after usual post-treatment to produce a compound represented by the formula (I-5-7).

The compound represented by the formula (I-5-7), 5% palladium carbon, and tetrahydrofuran were added to a reactor and stirred under a hydrogen pressure of 0.5 MPa. The catalyst was filtered off, and then purification was performed by column chromatography after usual post-treatment to produce a compound represented by the formula (I-5-8).

The compound represented by the formula (I-5-8), the compound represented by the formula (I-5-5), and ethanol were added to a reactor and stirred. Then, purification was performed by column chromatography after usual post-treatment to produce a compound represented by the formula (I-5-9).

The compound represented by the formula (I-5-9), 1,3-propanediol, triphenylphosphine, and tetrahydrofuran were added to a reactor. Then, diisopropyl azodicarboxylate was added dropwise and stirred under ice cooling. Then, purification was performed by column chromatography after usual post-treatment to produce a compound represented by the formula (I-5-10).

The compound represented by the formula (I-5-10), a compound represented by the formula (I-5-11), N,N-dimethylaminopyridine, and dichloromethane were added to a reactor. Then, diisopropyl carbodiimide was added dropwise and stirred. Then, purification was performed by column chromatography after usual post-treatment to produce a compound represented by the formula (I-5-12).

The compound represented by the formula (I-5-12) and dichloromethane were added to a reactor. Then, trifluoroacetic acid was added dropwise and stirred under ice cooling. Then, purification was performed by column chromatography after usual post-treatment to produce a compound represented by the formula (I-5-13).

The compound represented by the formula (I-5-13), the compound represented by the formula (I-5-2), N,N-dimethylaminopyridine, and dichloromethane were added to a reactor. Then, diisopropyl carbodiimide was added dropwise and stirred under ice cooling. Then, purification was performed by column chromatography and recrystallization after usual post-treatment to produce a compound represented by the formula (I-5).

MS (m/z): 842 [M⁺+1]

### Example 6

### Production of Compound Represented by Formula (I-6)

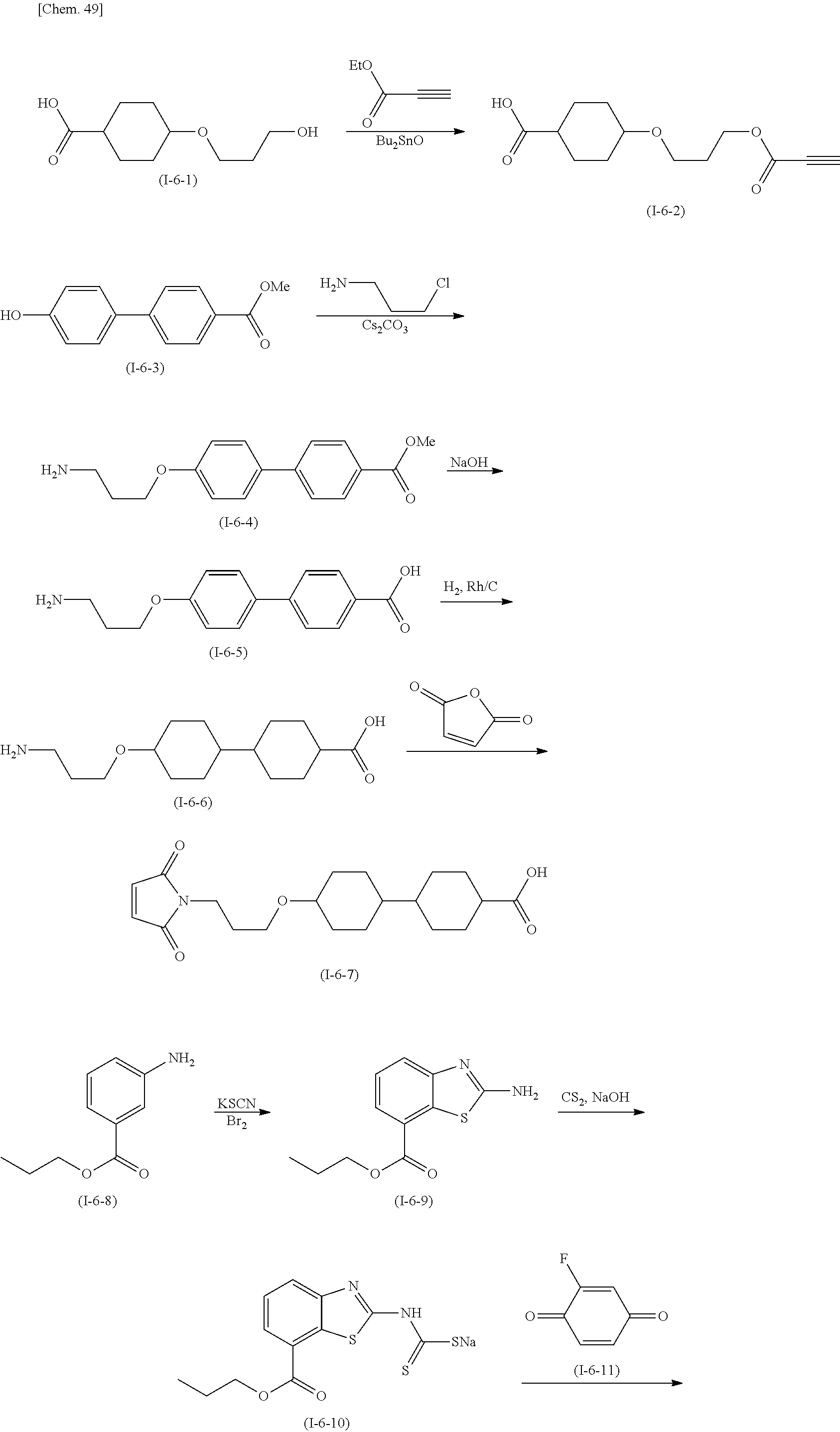

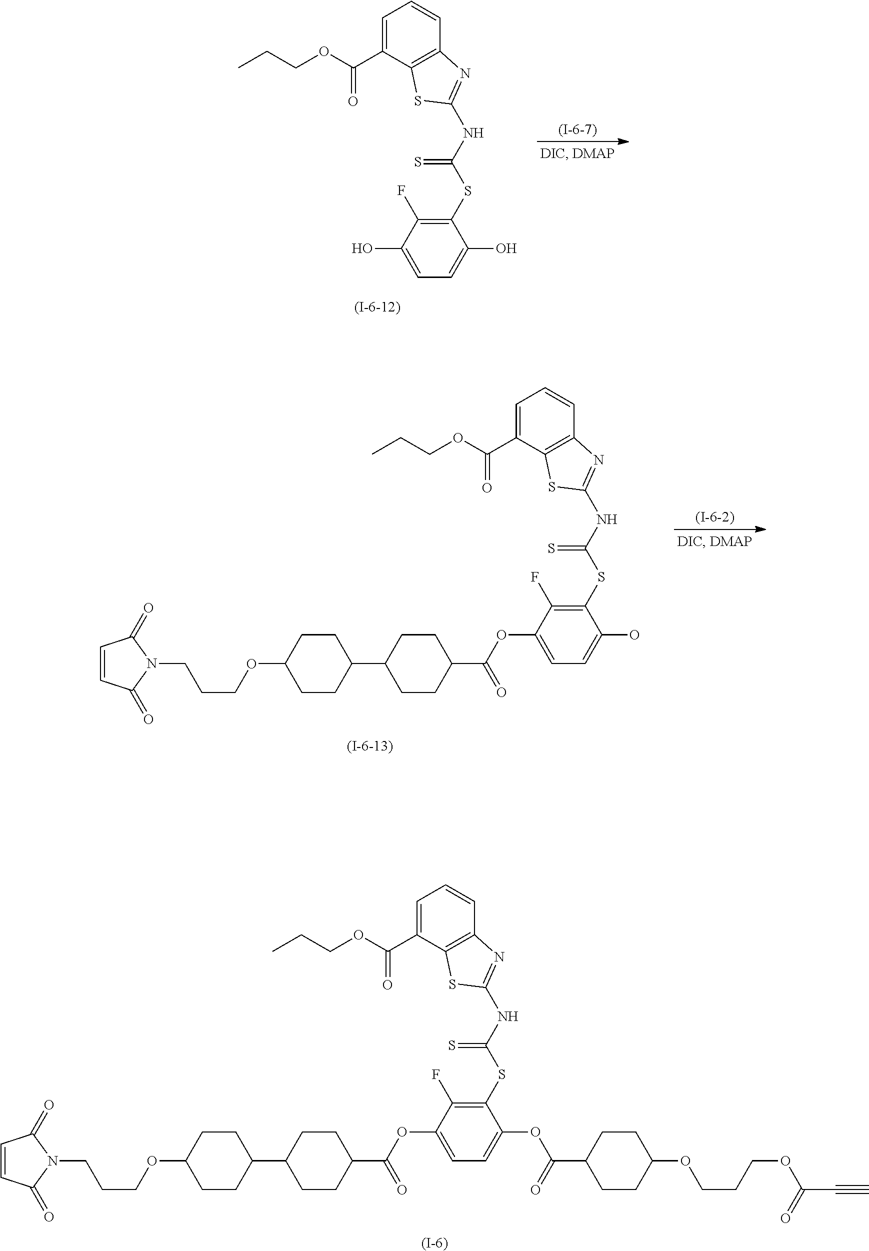 [SMILES: CCCOC(=O)c1cccc2nc(NC(=S)Sc3c(O)ccc(O)c3F)sc12 ; "(I-6-12)" ; CCCOC(=O)c1cccc2nc(NC(=S)Sc3c(O)ccc(OC(=O)C4CCC(C5CCC(OCCCN6C(=O)C=CC6=O)CC5)CC4)c3F)sc12 ; "(I-6-13)" ; C#CC(=O)OCCCOC1CCC(C(=O)Oc2ccc(OC(=O)C3CCC(C4CCC(OCCCN5C(=O)C=CC5=O)CC4)CC3)c(F)c2SC(=S)Nc2nc3cccc(C(=O)OCCC)c3s2)CC1 ; "(I-6)"]

A compound represented by the formula (I-6-1), toluene, ethyl propiolate, and dibutyltin oxide were added to a reactor and heated under reflux. Then, purification was performed by column chromatography after usual post-treatment to produce a compound represented by the formula (I-6-2).

A compound represented by the formula (I-6-3), 3-chloropropylamine, cesium carbonate, and dimethylsulfoxide were added to a reactor and heated and stirred. Then, purification was performed by column chromatography after usual post-treatment to produce a compound represented by the formula (I-6-4).

The compound represented by the formula (I-6-4), methanol, and an aqueous sodium hydroxide solution were added to a reactor and heated and stirred. Then, purification was performed by column chromatography and recrystallization after usual post-treatment to produce a compound represented by the formula (I-6-5).

The compound represented by the formula (I-6-5), acetic acid, and 5% rhodium carbon were added to a reactor and heated and stirred in a hydrogen atmosphere. The catalyst was removed, and then purification was performed by column chromatography and recrystallization after usual post-treatment to produce a compound represented by the formula (I-6-6).

The compound represented by the formula (I-6-6), maleic anhydride, and acetic acid were added to a reactor and heated and stirred. Then, purification was performed by column chromatography after usual post-treatment to produce a compound represented by the formula (I-6-7).

First, potassium thiocyanate and acetic acid were added to a reactor. A solution prepared by dissolving a compound represented by the formula (I-6-8) in acetic acid was added dropwise and stirred. Then, an acetic acid solution of bromine was added dropwise and stirred. Then, purification was performed by column chromatography after usual post-treatment to produce a compound represented by the formula (I-6-9).

The compound represented by the formula (I-6-9), N,N-dimethylformamide, carbon disulfide, and sodium hydroxide were added to a reactor and stirred. Then, chloroform was added to the resultant mixture, and the precipitate was filtered off and dried to produce a compound represented by the formula (I-6-10).

The compound represented by the formula (I-6-10), water, and methanol were added to a reactor. Then, in an inert atmosphere, an acetic acid-methanol solution of a compound represented by the formula (I-6-11) was added dropwise and stirred under cooling. Then, usual post-treatment was performed in an inert atmosphere to produce a compound represented by the formula (I-6-12).

In an inert atmosphere, the compound represented by the formula (I-6-12), the compound represented by the formula (I-6-7), dimethylaminopyridine, and dichloromethane were added to a reactor. Then, diisopropyl carbodiimide was added dropwise and stirred under cooling. Then, purification was performed by column chromatography after usual post-treatment to produce a compound represented by the formula (I-6-13).

The compound represented by the formula (I-6-13), the compound represented by the formula (I-6-2), dimethylaminopyridine, and dichloromethane were added to a reactor. Then, diisopropyl carbodiimide was added dropwise and stirred under cooling. Then, purification was performed by column chromatography and recrystallization after usual post-treatment to produce a compound represented by the formula (I-6).

MS (m/z): 1020 [M⁺+1]

### Example 7

### Production of Compound Represented by Formula (I-7)

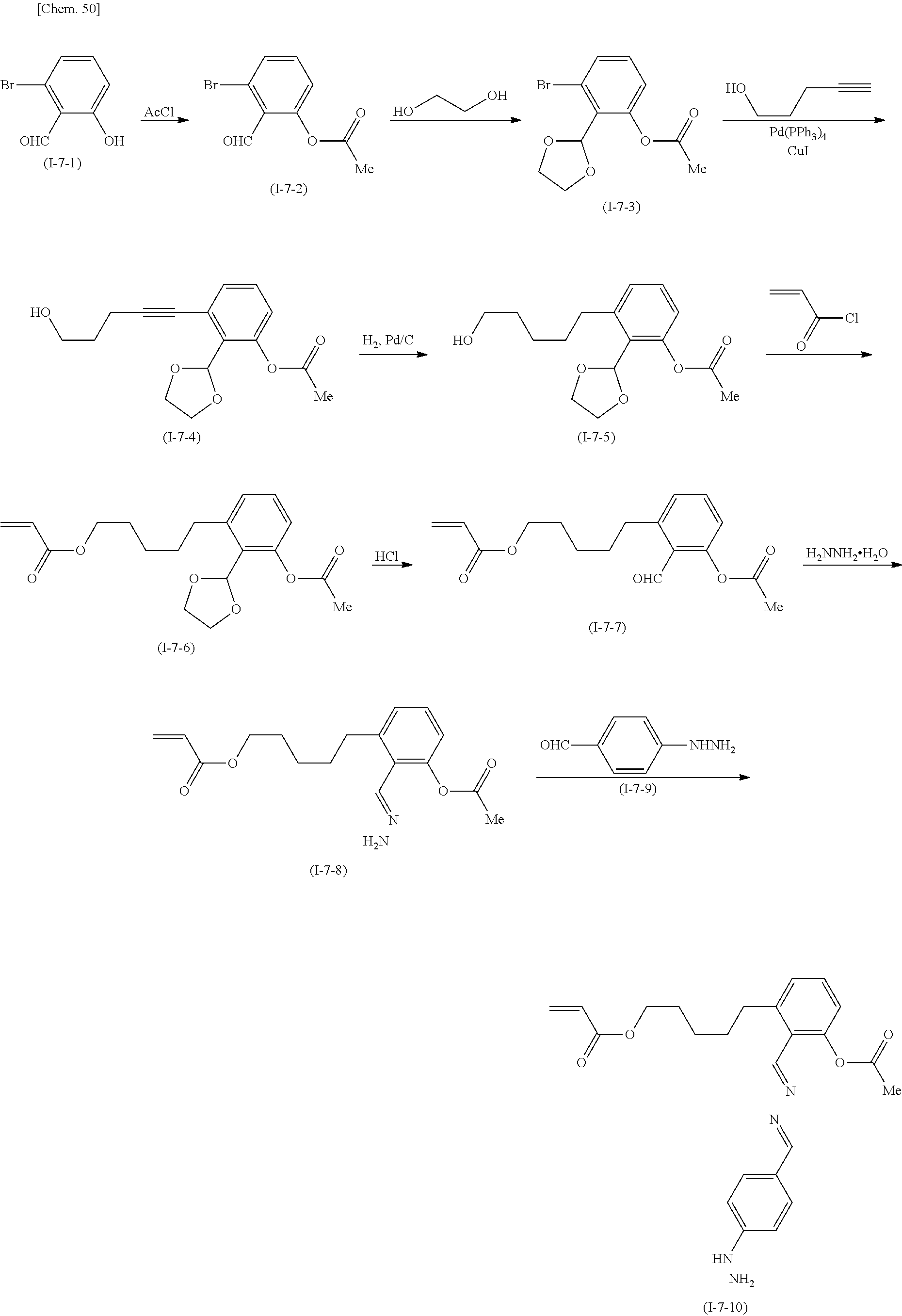

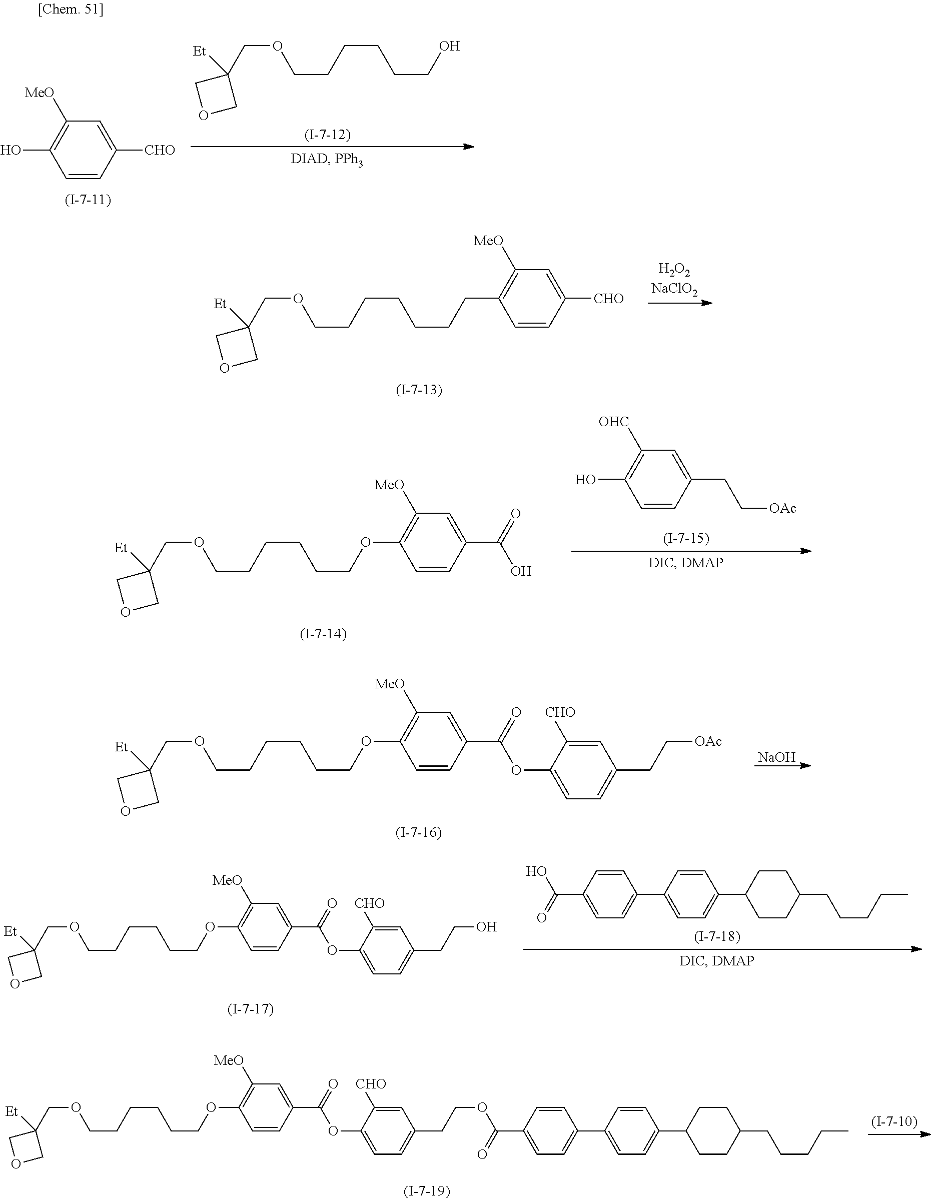

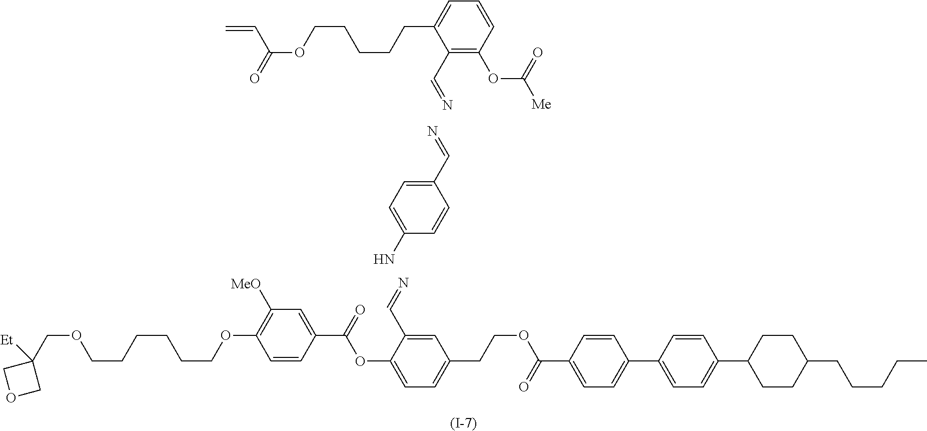

A compound represented by the formula (I-7-1), pyridine, and dichloromethane were added to a reactor. Then, a dichloromethane solution of acetyl chloride was added dropwise and stirred under ice cooling. Then, purification was performed by column chromatography after usual post-treatment to produce a compound represented by the formula (I-7-2).

The compound represented by the formula (i-7-2), ethylene glycol, toluene, and p-toluenesulfonic acid monohydrate were added to a reactor and heated and stirred. Then, purification was performed by column chromatography after usual post-treatment to produce a compound represented by the formula (I-7-3).

In an inert atmosphere, the compound represented by the formula (I-7-3), 4-pentyn-1-ol, copper(I) iodide, tetrakis(triphenylphosphine) palladium(0), N,N-dimethylformamide, and triethylamine were added to a reactor and heated and stirred. Then, purification was performed by column chromatography after usual post-treatment to produce a compound represented by the formula (I-7-4).

The compound represented by the formula (I-7-4), tetrahydrofuran, and 5% palladium carbon were added to a reactor and stirred in a hydrogen atmosphere. The catalyst was removed, and then purification was performed by column chromatography to produce a compound represented by the formula (I-7-5).

The compound represented by the formula (I-7-5), diisopropylethylamine, and dichloromethane were added to a reactor. Then, acryloyl chloride was added dropwise and stirred under ice cooling. Then, purification was performed by column chromatography after usual post-treatment to produce a compound represented by the formula (I-7-6).

The compound represented by the formula (I-7-6), tetrahydrofuran, and concentrated hydrochloric acid were added to a reactor. Then, purification was performed by column chromatography after usual post-treatment to produce a compound represented by the formula (I-7-7).

The compound represented by the formula (i-7-7), ethanol, and hydrazine monohydrate were added to a reactor. The resultant mixture was diluted with dichloromethane, and then purification was performed by column chromatography after usual post-treatment to produce a compound represented by the formula (I-7-8).

The compound represented by the formula (I-7-8), a compound represented by the formula (I-7-9), and ethanol were added to a reactor and stirred. The resultant mixture was diluted with dichloromethane, and then purification was performed by column chromatography after usual post-treatment to produce a compound represented by the formula (I-7-10).

A compound represented by the formula (I-7-12) was produced by the method described in Macromolecular Chemistry and Physics, 2009, Vol. 210, No. 7, pp. 531-541. A compound represented by the formula (I-7-11), the compound represented by the formula (I-7-12), tetrahydrofuran, triphenylphosphine were added to a reactor. Then, diisopropyl azodicarboxylate was added dropwise and stirred under ice cooling. Then, purification was performed by column chromatography after usual post-treatment to produce a compound represented by the formula (I-7-13).

The compound represented by the formula (I-7-13), methanol, water, sodium dihydrogen phosphate dihydrate, sodium chlorite, and hydrogen peroxide were added to a reactor and heated and stirred. Then, water was added to the resultant mixture, and the precipitate was filtered off and dried to produce a compound represented by the formula (I-7-14).

The compound represented by the formula (I-7-14), a compound represented by the formula (I-7-15), N,N-dimethylaminopyridine, and dichloromethane were added to a reactor. Then, diisopropyl carbodiimide was added dropwise and stirred under ice cooling. Then, purification was performed by column chromatography after usual post-treatment to produce a compound represented by the formula (I-7-16).

The compound represented by the formula (I-7-16), methanol, and an aqueous sodium hydroxide solution were added to a reactor and heated and stirred. Then, purification was performed by column chromatography after usual post-treatment to produce a compound represented by the formula (I-7-17).

The compound represented by the formula (I-7-17), a compound represented by the formula (I-7-18), N,N-dimethylaminopyridine, and dichloromethane were added to a reactor. Then, diisopropyl carbodiimide was added dropwise and stirred under ice cooling. Then, purification was performed by column chromatography after usual post-treatment to produce a compound represented by the formula (I-7-19).

The compound represented by the formula (I-7-19), the compound represented by the formula (I-7-10), (±)-10-camphorsulfonic acid, ethanol, and tetrahydrofuran were added to a reactor and stirred. The precipitate was filtered off, and then purification was performed by column chromatography and re-crystallization to produce a compound represented by the formula (I-7).

MS (m/z): 1265 [M⁺+1]

### Example 8

### Production of Compound Represented by Formula (I-8)

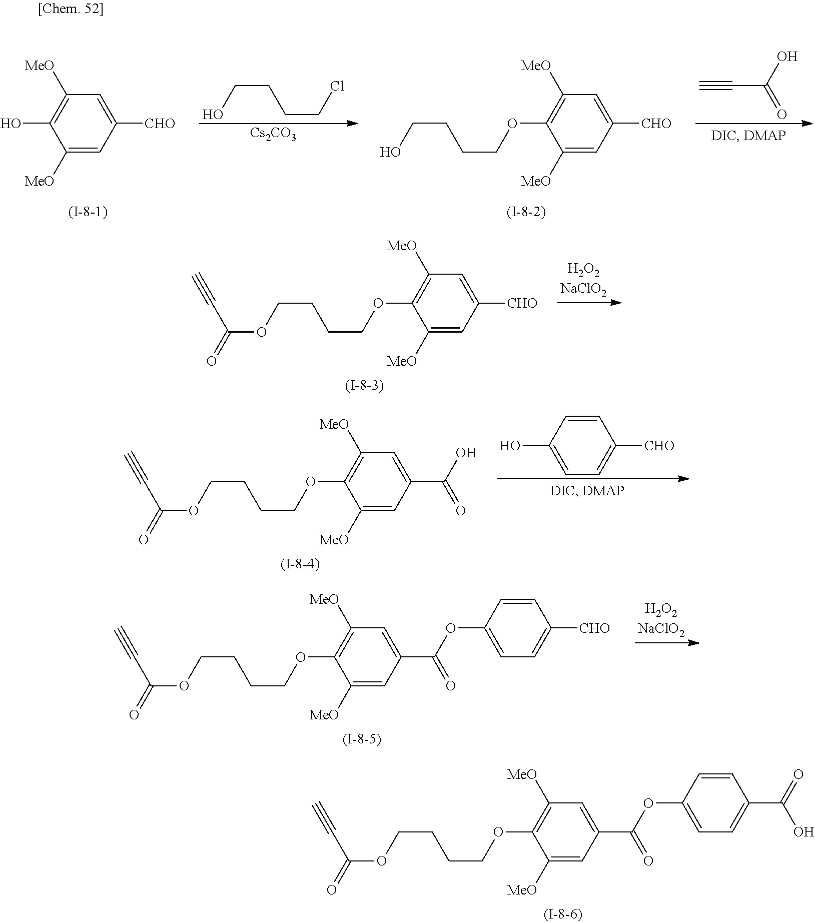

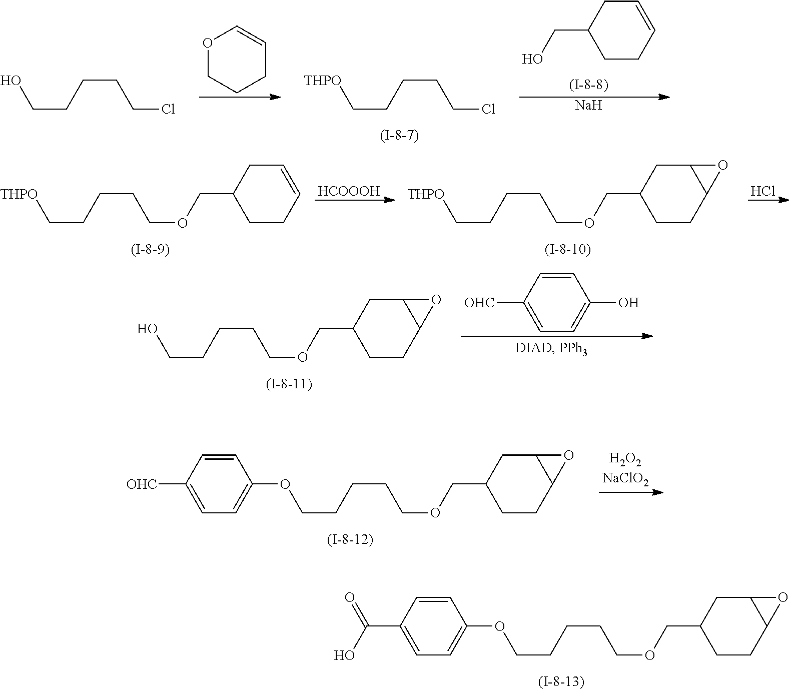

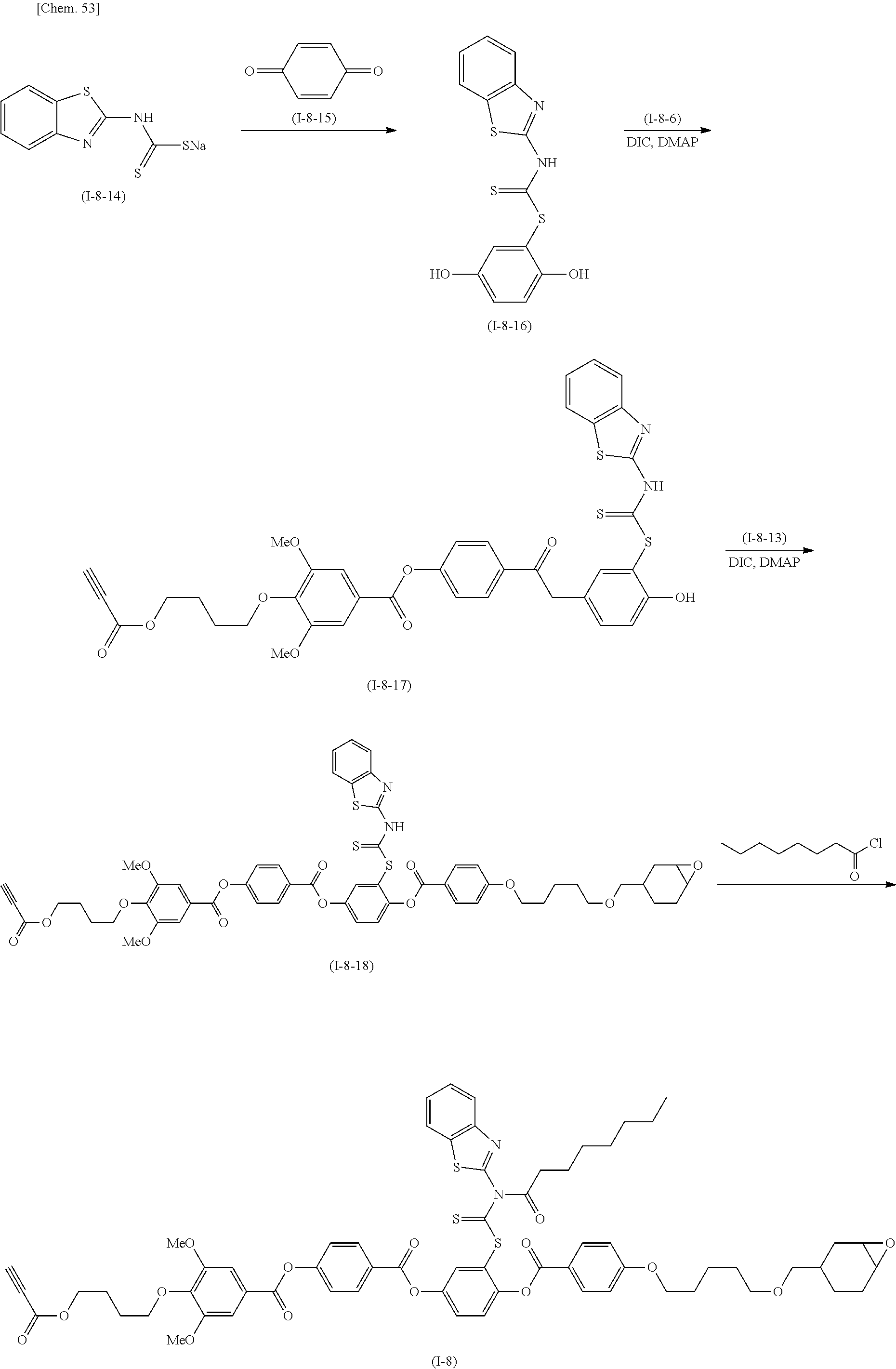

A compound represented by the formula (I-8-1), propiolic acid, 4-chlorobutanol, cesium carbonate, and dimethylsulfoxide were added to a reactor and heated and stirred. Then, purification was performed by column chromatography after usual post-treatment to produce a compound represented by the formula (I-8-2).

The compound represented by the formula (I-8-2), N,N-dimethylaminopyridine, and dichloromethane were added to a reactor. Then, diisopropyl carbodiimide was added dropwise and stirred under ice cooling. Then, purification was performed by column chromatography after usual post-treatment to produce a compound represented by the formula (I-8-3).

The compound represented by the formula (I-8-3), sodium dihydrogen phosphate dihydrate, methanol, water, sodium chlorite, and an aqueous hydrogen peroxide solution were added to a reactor and heated and stirred. The resultant mixture was diluted with ethyl acetate, and then purification was performed by column chromatography after usual post-treatment to produce a compound represented by the formula (I-8-4).

The compound represented by the formula (I-8-4), 4-hydroxybenzaldehyde, N,N-dimethylaminopyridine, and dichloromethane were added to a reactor. Then, diisopropyl carbodiimide was added dropwise and stirred under ice cooling. Then, purification was performed by column chromatography after usual post-treatment to produce a compound represented by the formula (I-8-5).

The compound represented by the formula (I-8-5), sodium dihydrogen phosphate dihydrate, methanol, water, sodium chlorite, and an aqueous hydrogen peroxide solution were added to a reactor and heated and stirred. The resultant mixture was diluted with ethyl acetate, and then purification was performed by column chromatography after usual post-treatment to produce a compound represented by the formula (I-8-6).

First, 5-chloropentanol, pyridinium p-toluenesulfonate, and dichloromethane were added to a reactor. Then, 3,4-dihydro-2H-pyrane was added dropwise and stirred. Then, purification was performed by column chromatography after usual post-treatment to produce a compound represented by the formula (I-8-7).

A compound represented by the formula (I-8-8), tetrahydrofuran, and sodium hydride were added to a reactor and stirred. A tetrahydrofuran solution of the compound represented by the formula (I-8-7) was added dropwise and heated and stirred. Water was added to the mixture, and then purification was performed by column chromatography after usual post-treatment to produce a compound represented by the formula (I-8-9).

First, formic acid and hydrogen peroxide were added to a reactor and stirred. A dichloromethane solution of the compound represented by the formula (I-8-9) was added dropwise and heated and stirred. Then, purification was performed by column chromatography after usual post-treatment to produce a compound represented by the formula (I-8-10).

The compound represented by the formula (I-8-10), methanol, tetrahydrofuran, and concentrated hydrochloric acid were added to a reactor and heated and stirred. Then, purification was performed by column chromatography after usual post-treatment to produce a compound represented by the formula (I-8-11).

The compound represented by the formula (I-8-11), 4-hydroxybenzaldehyde, triphenylphosphine, and tetrahydrofuran were added to a reactor. Then, diisopropyl azodicarboxylate was added dropwise and stirred under ice cooling. Then, purification was performed by column chromatography after usual post-treatment to produce a compound represented by the formula (I-8-12).

The compound represented by the formula (I-8-12), sodium dihydrogen phosphate dihydrate, methanol, water, sodium chlorite, and an aqueous hydrogen peroxide solution were added to a reactor and heated and stirred. The resultant mixture was diluted with ethyl acetate, and then purification was performed by column chromatography after usual post-treatment to produce a compound represented by the formula (I-8-13).

A compound represented by the formula (I-8-14) was produced by the method described in European Journal of Organic Chemistry, 2004, No. 20, pp. 4203-4214. The compound represented by the formula (I-8-14), water, and methanol were added to a reactor. Then, in an inert atmosphere, an acetic acid-methanol solution of a compound represented by the formula (I-8-15) was added dropwise and stirred under ice cooling. Then, in an inert atmosphere, post-treatment was performed to produce a compound represented by the formula (I-8-16).

In an inert atmosphere, the compound represented by the formula (I-8-16), the compound represented by the formula (I-8-6), N,N-dimethylaminopyridine, and dichloromethane were added to a reactor. Then, diisopropyl carbodiimide was added dropwise and stirred under ice cooling. Then, purification was performed by column chromatography and recrystallization after usual post-treatment to produce a compound represented by the formula (I-8-17).

The compound represented by the formula (I-8-17), the compound represented by the formula (I-8-13), N,N-dimethylaminopyridine, and dichloromethane were added to a reactor. Then, diisopropyl carbodiimide was added dropwise and stirred under ice cooling. Then, purification was performed by column chromatography and recrystallization after usual post-treatment to produce a compound represented by the formula (I-8-18).

The compound represented by the formula (I-8-18), dichloromethane, and trimethylamine were added to a reactor. Then, a dichloromethane solution of octanoyl chloride was added dropwise and heated and stirred. Then, purification was performed by column chromatography and recrystallization after usual post-treatment to produce a compound represented by the formula (I-8).

MS (m/z): 1201 [M⁺+1]

### Example 9

### Production of Compound Represented by Formula (I-9)

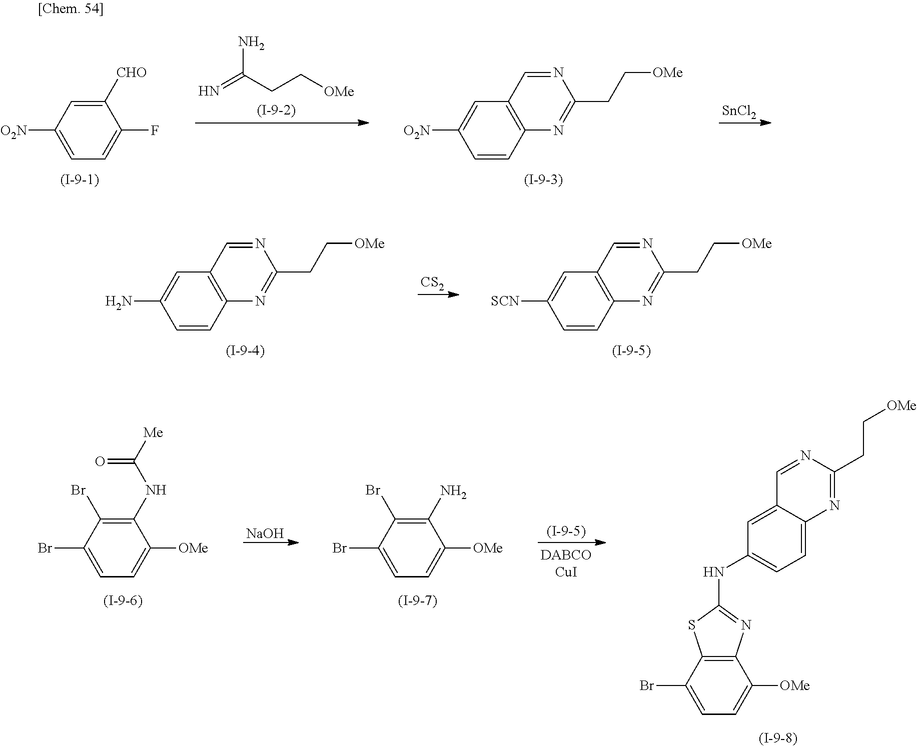

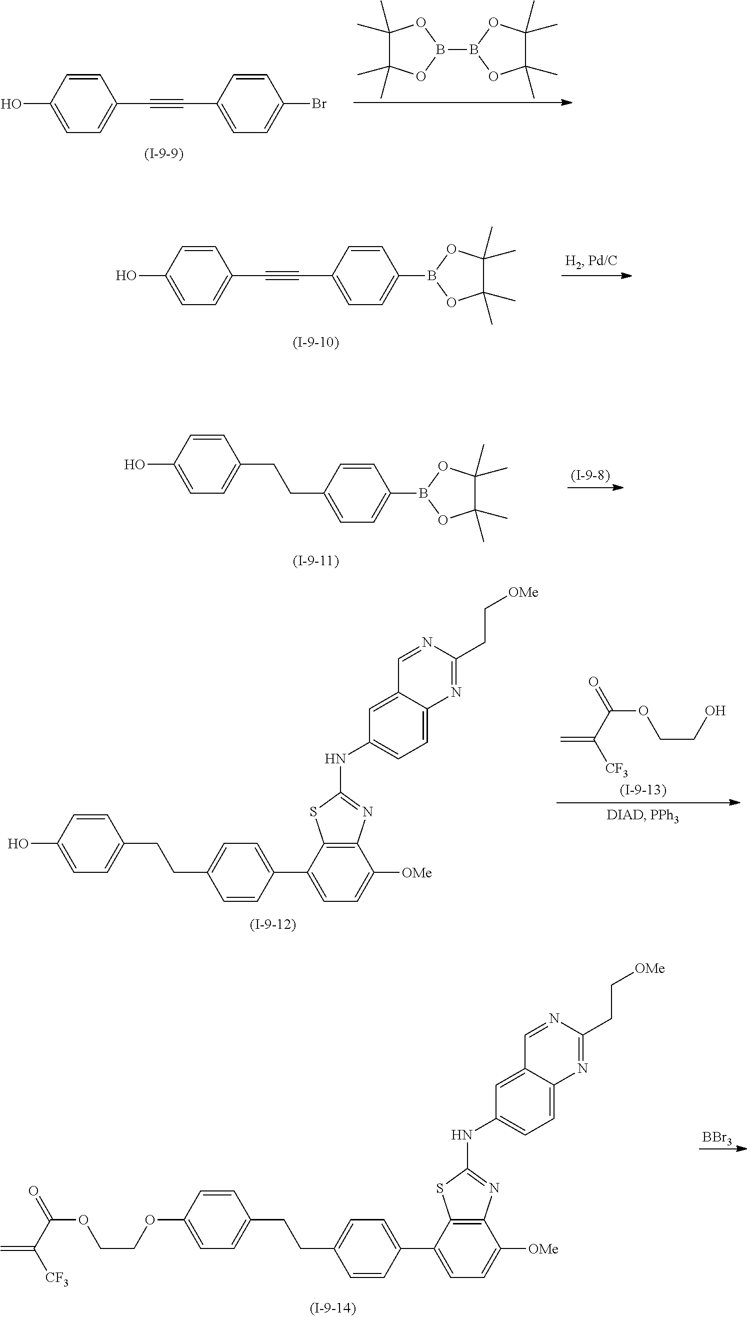

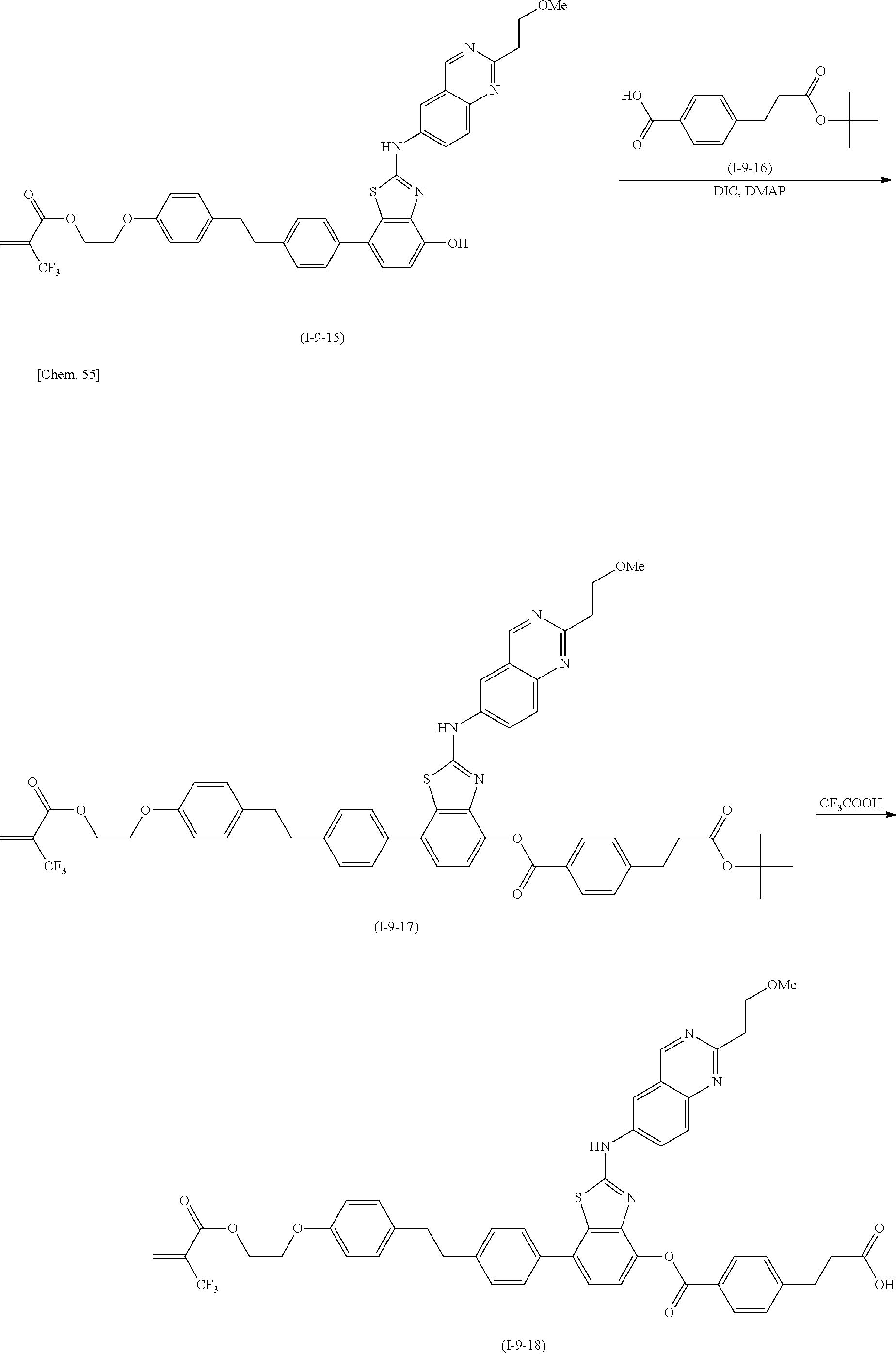

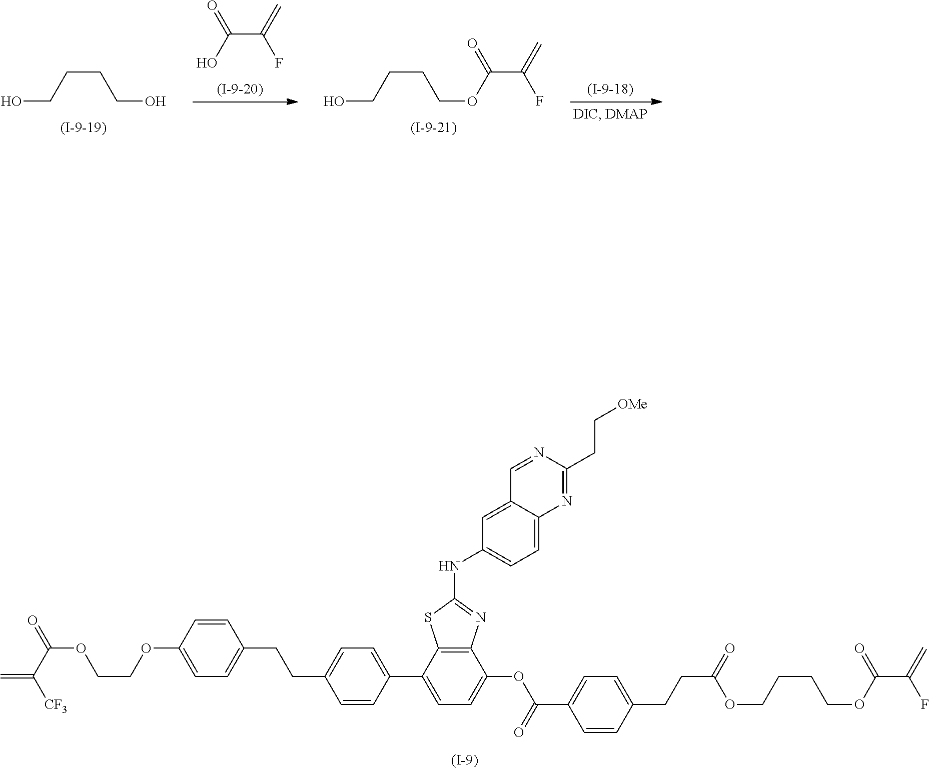

A compound represented by the formula (I-9-2), acetonitrile, potassium carbonate, and a compound represented by the formula (I-9-1) were added to a reactor and heated and stirred. Then, purification was performed by column chromatography after usual post-treatment to produce a compound represented by the formula (I-9-3).

The compound represented by the formula (I-9-3), methanol, tin(II) chloride, and concentrated hydrochloric acid were added to a reactor and stirred. The reaction solution was poured into an aqueous sodium bicarbonate solution, and then purification was performed by column chromatography after usual post-treatment to produce a compound represented by the formula (I-9-4).

The compound represented by the formula (I-9-4), triethylamine, and carbon disulfide were added to a reactor and stirred. Then, an ethanol solution of di-tert-butyl dicarbonate and 1,4-diazabicyclo[2.2.2]octane were added and stirred under ice cooling. The solvent was distilled off to produce a compound represented by the formula (I-9-5).

A compound represented by the formula (I-9-6), methanol, and an aqueous sodium hydroxide solution were added to a reactor and heated and stirred. Then, purification was performed by column chromatography after usual post-treatment to produce a compound represented by the formula (I-9-7).

The compound represented by the formula (I-9-7), 1,4-diazabicyclo[2.2.2]octane, copper iodide, 1,10-phenanthroline, and toluene were added to a reactor. A toluene solution of the compound represented by the formula (I-9-5) was added dropwise and heated and stirred. Then, purification was performed by column chromatography and recrystallization after usual post-treatment to produce a compound represented by the formula (I-9-8).

In an inert atmosphere, a compound represented by the formula (I-9-9), bispinacolatodiboron, potassium acetate, dichlorobis(triphenylphosphine) palladium (II), and dimethylsulfoxide were added to a reactor and heated and stirred. Then, usual post-treatment was performed to produce a compound represented by the formula (I-9-10).

The compound represented by the formula (I-9-10), tetrahydrofuran, 5% palladium carbon were added to a reactor and stirred in a hydrogen atmosphere. The catalyst was removed and then, the solvent was distilled off to produce a compound represented by the formula (I-9-11).

In an inert atmosphere, the compound represented by the formula (I-9-11), the compound represented by the formula (I-9-8), potassium carbonate, ethanol, and tetrakis(triphenylphosphine)palladium(0) were added to a reactor and heated and stirred. Then, purification was performed by column chromatography and recrystallization after usual post-treatment to produce a compound represented by the formula (I-9-12).

The compound represented by the formula (I-9-12), a compound represented by the formula (I-9-13), triphenylphosphine, and tetrahydrofuran were added to a reactor and heated. Then, diisopropyl azodicarboxylate was added and stirred under ice cooling. Then, purification was performed by column chromatography and recrystallization after usual post-treatment to produce a compound represented by the formula (I-9-14).

The compound represented by the formula (I-9-14) and dichloromethane were added to a reactor. The resultant mixture was cooled to −78° C., and boron tribromide was added dropwise and stirred. The reaction solution was poured into water, and then purification was performed by column chromatography and recrystallization after usual post-treatment to produce a compound represented by the formula (I-9-15).

The compound represented by the formula (I-9-15), a compound represented by the formula (I-9-16), N,N-dimethylaminopyridine, and dichloromethane were added to a reactor. Then, diisopropyl carbodiimide was added dropwise and stirred under ice cooling. Then, purification was performed by column chromatography and recrystallization after usual post-treatment to produce a compound represented by the formula (I-9-17).

The compound represented by the formula (I-9-17) and dichloromethane were added to a reactor. Then, trifiuoroacetic acid was added and stirred under ice cooling. The reaction solution was poured into water, and then purification was performed by column chromatography and recrystallization after usual post-treatment to produce a compound represented by the formula (I-9-18).

A compound represented by the formula (I-9-19), a compound represented by the formula (I-9-20), N,N-dimethylaminopyridine, and dichloromethane were added to a reactor. Then, diisopropyl carbodiimide was added dropwise and stirred under ice cooling. Then, purification was performed by column chromatography after usual post-treatment to produce a compound represented by the formula (I-9-21).

The compound represented by the formula (I-9-21), the compound represented by the formula (I-9-18), N,N-dimethylaminopyridine, and dichloromethane were added to a reactor. Then, diisopropyl carbodiimide was added dropwise and stirred under ice cooling. Then, purification was performed by column chromatography and recrystallization after usual post-treatment to produce a compound represented by the formula (I-9).

MS (m/z): 1035 M⁺+1]

### Example 10

### Production of Compound Represented by Formula (I-10)

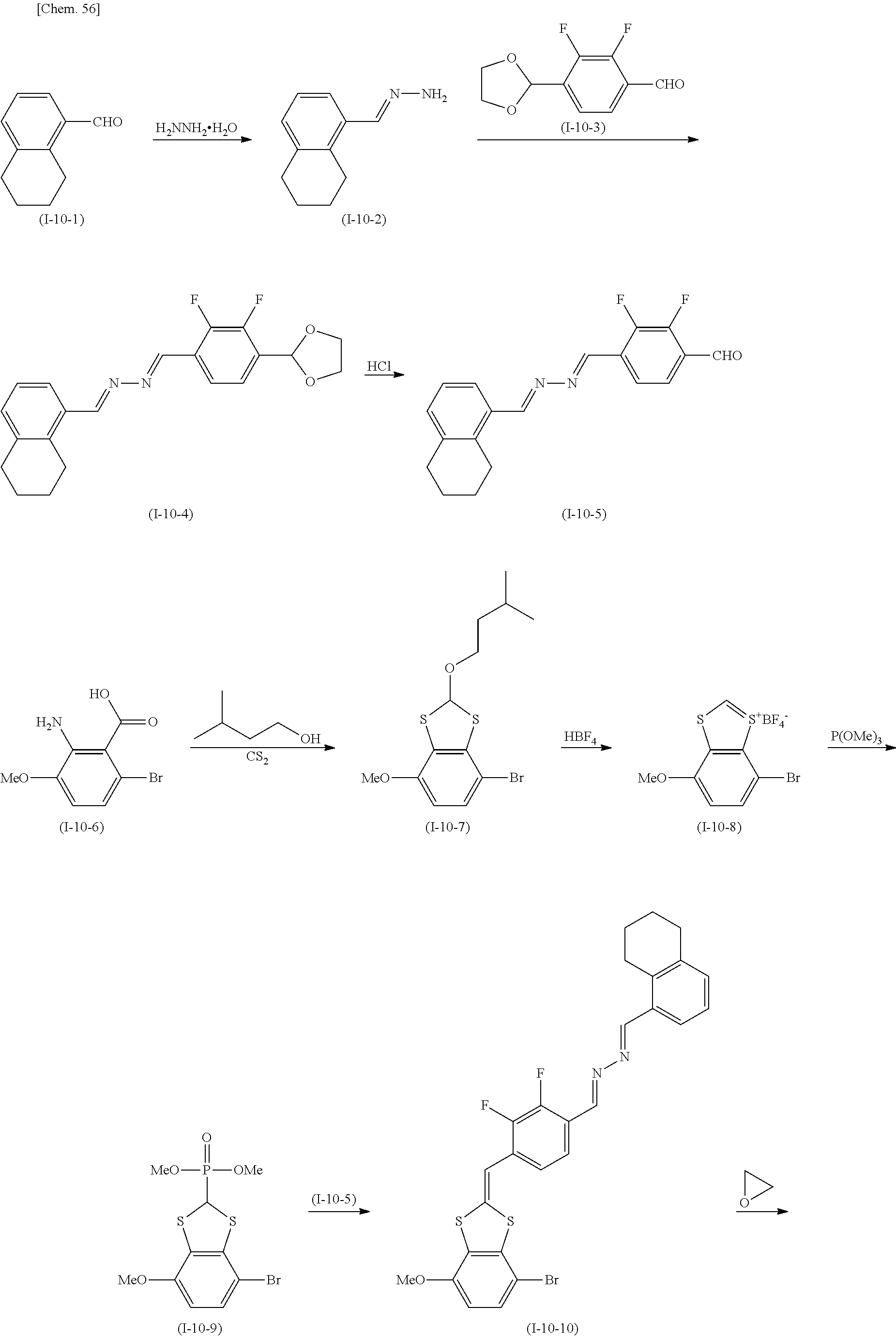

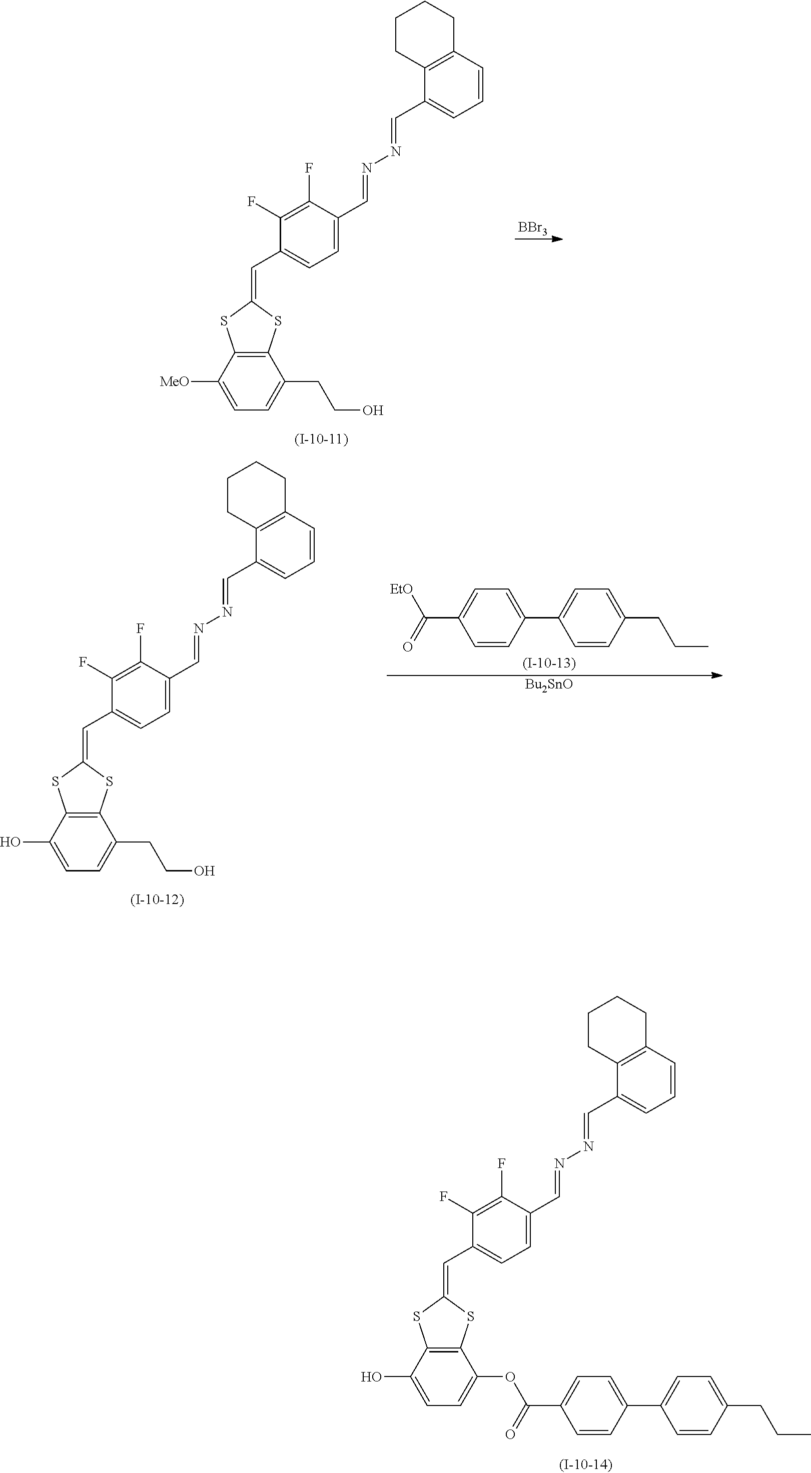 [SMILES: "Bu2SnO" ; COc1ccc(CCO)c2c1S/C(=C/c1ccc(/C=N/N=C/c3cccc4c3CCCC4)c(F)c1F)S2 ; "BBr3" ; OCCc1ccc(O)c2c1S/C(=C\c1ccc(/C=N/N=C/c3cccc4c3CCCC4)c(F)c1F)S2 ; CCCc1ccc(-c2ccc(C(=O)OCC)cc2)cc1 ; CCCc1ccc(-c2ccc(C(=O)Oc3ccc(O)c4c3S/C(=C\c3ccc(/C=N/N=C/c5cccc6c5CCCC6)c(F)c3F)S4)cc2)cc1]

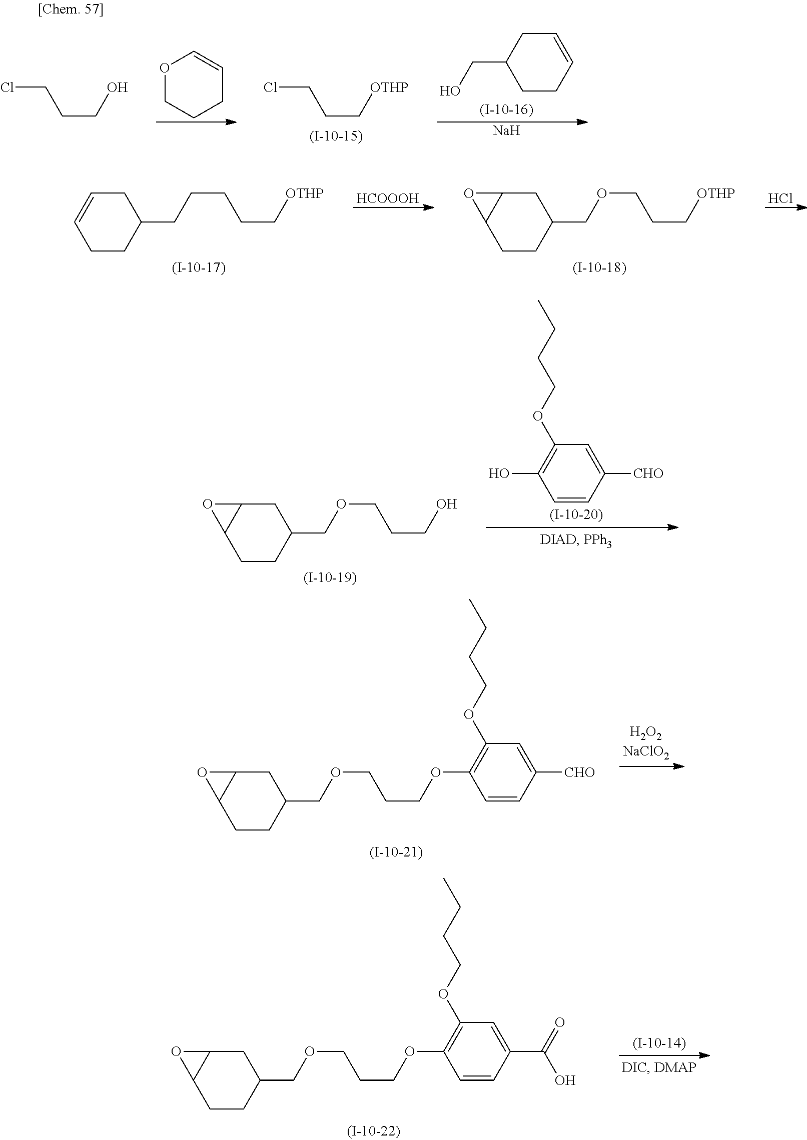

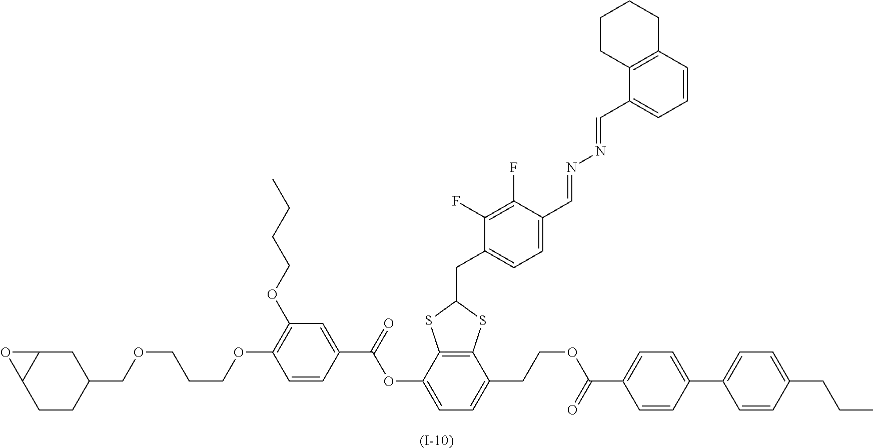

A compound represented by the formula (I-10-1) and ethanol were added to a reactor. Then, hydrazine monohydrate was added dropwise and stirred. Then, purification was performed by column chromatography after usual post-treatment to produce a compound represented by the formula (I-10-2).

A compound represented by the formula (I-10-3) and ethanol were added to a reactor. An ethanol solution of the compound represented by the formula (I-10-2) was added dropwise and stirred. Then, purification was performed by column chromatography after usual post-treatment to produce a compound represented by the formula (I-10-4).

The compound represented by the formula (I-10-4), tetrahydrofuran, and concentrated hydrochloric acid were added to a reactor and heated and stirred. Then, purification was performed by column chromatography after usual post-treatment to produce a compound represented by the formula (I-10-5).

First, isoamyl nitrate, 3-methylbutanol, carbon disulfide, and 1,2-dichlorethane were added to a reactor. A compound represented by the formula (I-10-6) was added and heated and stirred. Then, purification was performed by column chromatography after usual post-treatment to produce a compound represented by the formula (I-10-7).

The compound represented by the formula (I-10-7) and acetic anhydride were added to a reactor. Then, tetrafluoroboric acid was added and stirred under ice cooling. Then, diethyl ether was added and the precipitated solid was filtered off and dried to produce a compound represented by the formula (I-10-8).

The compound represented by the formula (I-10-8) and acetonitrile were added to a reactor. Then, trimethyl phosphite and sodium iodide were added and stirred. The solvent was distilled off, water was added, and then the resultant solid was filtered off and dried to produce a compound represented by the formula (I-10-9).

The compound represented by the formula (I-10-9) and tetrahydrofuran were added to a reactor. The resultant mixture was cooled to −78° C., and a hexane solution of butyl lithium was added dropwise and stirred. Then, a tetrahydrofuran solution of the compound represented by the formula (I-10-5) was added dropwise and stirred. Then, purification was performed by column chromatography and recrystallization after usual post-treatment to produce a compound represented by the formula (I-10-10).

The compound represented by the formula (I-10-10) and tetrahydrofuran were added to a reactor. The resultant mixture was cooled to −78° C., and a hexane solution of butyl lithium was added dropwise and stirred. Then, a tetrahydrofuran solution of ethylene oxide was added dropwise and stirred. Then, purification was performed by column chromatography and recrystallization after usual post-treatment to produce a compound represented by the formula (I-10-11).

The compound represented by the formula (I-10-11) and dichloromethane were added to a reactor. The resultant mixture was cooled to −78° C., and boron tribromide was added dropwise and stirred. The reaction solution was poured into water, and then purification was performed by column chromatography and recrystallization after usual post-treatment to produce a compound represented by the formula (I-10-12).

The compound represented by the formula (I-10-12), a compound represented by the formula (I-10-13), dibutyltin oxide, and toluene were added to a reactor and heated and stirred. Then, purification was performed by column chromatography after usual post-treatment to produce a compound represented by the formula (I-10-14).

First, 3-chloropropanol, pyridinium p-toluenesulfonate, and dichloromethane were added to a reactor. Then, 3,4-dihydro-2H-pyrane was added dropwise and stirred. Then, purification was performed by column chromatography after usual post-treatment to produce a compound represented by the formula (I-10-15).

A compound represented by the formula (I-10-16), tetrahydrofuran, and sodium hydride were added to a reactor and stirred. A tetrahydrofuran solution of the compound represented by the formula (I-10-15) was added dropwise and heated and stirred. Then, water was added dropwise, and purification was performed by column chromatography after usual post-treatment to produce a compound represented by the formula (I-10-17).

First, formic acid and hydrogen peroxide were added to a reactor. A dichloromethane solution of the compound represented by the formula (I-10-17) was added dropwise and heated and stirred. Then, purification was performed by column chromatography after usual post-treatment to produce a compound represented by the formula (I-10-18).

The compound represented by the formula (I-10-18), methanol, tetrahydrofuran, and concentrated hydrochloric acid were added to a reactor and heated and stirred. Then, purification was performed by column chromatography after usual post-treatment to produce a compound represented by the formula (I-10-19).

The compound represented by the formula (I-10-19), a compound represented by the formula (I-10-20), triphenylphosphine, and tetrahydrofuran were added to a reactor. Then, diisopropyl azodicarboxylate was added dropwise and stirred under ice cooling. Then, purification was performed by column chromatography after usual post-treatment to produce a compound represented by the formula (I-10-21).

The compound represented by the formula (I-10-21), sodium dihydrogen phosphate dihydrate, methanol, water, sodium hydrochlorite, and an aqueous hydrogen peroxide solution were added to a reactor and heated and stirred. The reaction solution was diluted with ethyl acetate, and then purification was performed by column chromatography after usual post-treatment to produce a compound represented by the formula (I-10-22).

The compound represented by the formula (I-10-22), the compound represented by the formula (I-10-14), N,N-dimethylaminopyridine, and dichloromethane were added to a reactor. Then, diisopropyl carbodiimide was added dropwise and stirred under ice cooling. Then, purification was performed by column chromatography and recrystallization after usual post-treatment to produce a compound represented by the formula (I-10).

MS (m/z): 1105 M⁺+1]

### Examples 11 to 30 and Comparative Examples 1 to 4

The compounds represented by the formula (I-1) to the formula (I-10) in Example 1 to Example 10, a compound (R-1) described in Patent Literature 1, and a compound (R-2) described in Patent Literature 2 were used as compounds to be evaluated.

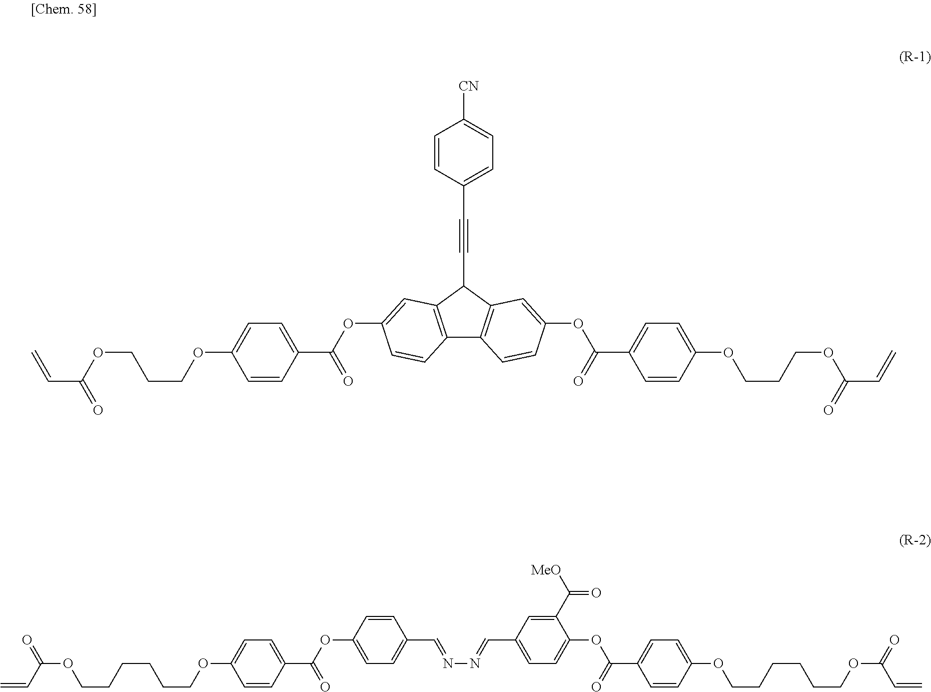

A liquid crystal composition containing 30% of a compound (X-1) described in Japanese Unexamined Patent Application Publication No. 2002-030042, 30% of a compound (X-2) described in Japanese Unexamined Patent Application Publication (Translation of PCT Application) No. 11-513019, and 40% of a compound (X-3) described in Japanese Unexamined Patent Application Publication No. 10-87565 was used as a mother liquid crystal (X).

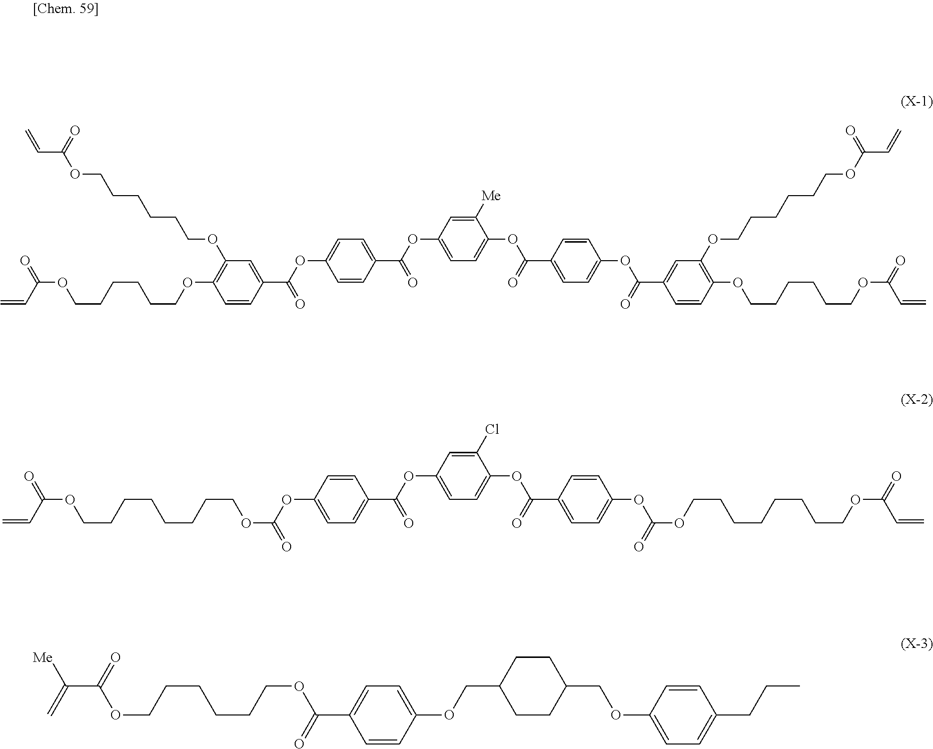

A polyimide solution for an alignment film was applied by a spin coating method on a glass substrate having a thickness of 0.7 mm, dried at 100° C. for 10 minutes, and then fired at 200° C. for 60 minutes to produce a coating film. The resultant coating film was rubbed. Rubbing was performed by using a commercial rubbing apparatus.

A coating solution was prepared by adding 1% of photopolymerization initiator Irgacure 907 (manufactured by BASF Corporation), 0.1% of 4-methoxyphenol, and 80% of chloroform to a composition prepared by adding 30% of each of the compounds to be evaluated to the mother liquid crystal (X). The resultant coating solution was applied to the rubbed glass substrate by a spin coating method. The coating solution was dried at 80° C. for 1 minute and further dried at 120° C. for 1 minute. Then, the substrate was irradiated with ultraviolet light for 25 seconds by using a high-pressure mercury lamp with an intensity of 40 mW/cm², thereby producing a film to be evaluated. Any of the resultant films was horizontally aligned. A correspondence relation between the film to be evaluated and each of the used compounds to be evaluated is shown in a table below.

TABLE 1

| Example | Film to be evaluated | Compound to be evaluated |
|---|---|---|
| Example 11 | Optically anisotropic body (XI-1) | Compound (I-1) of this invention |
| Example 12 | Optically anisotropic body (XI-2) | Compound (I-2) of this inventon |
| Example 13 | Optically anisotropic body (XI-3) | Compound (I-3) of this invention |
| Example 14 | Optically anisotropic body (XI-4) | Compound (I-4) of this invention |
| Example 15 | Optically anisotropic body (XI-5) | Compound (I-5) of this invention |
| Example 16 | Optically anisotropic body (XI-6) | Compound (I-6) of this invention |
| Example 17 | Optically anisotropic body (XI-7) | Compound (I-7) of this invention |
| Example 18 | Optically anisotropic body (XI-8) | Compound (I-8) of this invention |
| Example 19 | Optically anisotropic body (XI-9) | Compound (I-9) of this invention |
| Example 20 | Optically anisotropic body (XI-10) | Compound (I-10) of this invention |
| Comparative | Optically anisotropic body (XI-R-1) | Comparative Compound (R-1) |
| Example 1 | | |
| Comparative | Optically anisotropic body (XI-R-2) | Comparative Compound (R-2) |
| Example 2 | | |

The resultant film to be evaluated was measured with respect to an absorption maximum wavelength λomax in an in-plane direction perpendicular to the alignment direction. Measurement was performed by using a spectrophotometer (V-560 manufactured by JASCO Corporation), and the film to be evaluated was held between two polarization plates and arranged so that the alignment direction of the film to be evaluated was perpendicular to the polarization direction of the polarization plates (refer to the drawing). Also, the film to be evaluated was arranged so that the alignment direction of the film to be evaluated was perpendicular to the polarization direction of the polarization plates, and absorbance Ao in the in-plane direction perpendicular to the alignment direction at the wavelength λomax was measured. Similarly, the film to be evaluated was arranged so that the alignment direction of the film to be evaluated was parallel to the polarization direction of the polarization plates, and absorbance Ae in the direction parallel to the alignment direction at the wavelength λomax was measured. In addition, Ao/Ae was calculated from the obtained Ao and Ae. The results are shown in a table below.

TABLE 2

| | | λomax | |
|---|---|---|---|
| Example | Film to be evaluated | (nm) | Ao/Ae |
| Example 11 | Optically anisotropic body (XI-1) | 345 | 1.13 |
| Example 12 | Optically anisotropic body (XI-2) | 365 | 1.01 |
| Example 13 | Optically anisotropic body (XI-3) | 332 | 1.12 |
| Example 14 | Optically anisotropic body (XI-4) | 378 | 1.11 |
| Example 15 | Optically anisotropic body (XI-5) | 405 | 1.01 |
| Example 16 | Optically anisotropic body (XI-6) | 320 | 1.02 |
| Example 17 | Optically anisotropic body (XI-7) | 420 | 1.01 |
| Example 18 | Optically anisotropic body (XI-8) | 321 | 0.88 |
| Example 19 | Optically anisotropic body (XI-9) | 350 | 1.01 |
| Example 20 | Optically anisotropic body (XI-10) | 418 | 1.05 |
| Comparative | Optically anisotropic body (XI-R-1) | 315 | 1.02 |
| Example 1 | | | |
| Comparative | Optically anisotropic body (XI-R-2) | 310 | 0.76 |
| Example 2 | | | |

Next, heat resistance and light resistance of each of the films to be evaluated were evaluated. A test was performed by light irradiation for 600 hours using an accelerated weathering test machine (light source: xenon lamp, temperature: BPT 65° C., humidity: 50% RH, intensity 200 W/m²). A correspondence relation between the film to be evaluated and each of the used compounds to be evaluated is shown in a table below.

TABLE 3

| Example | Film to be evaluated | Compound to be evaluated |
|---|---|---|
| Example 21 | Optically anisotropic body (XII-1) | Compound (I-1) of this invention |
| Example 22 | Optically anisotropic body (XII-2) | Compound (I-2) of this invention |
| Example 23 | Optically anisotropic body (XII-3) | Compound (I-3) of this invention |
| Example 24 | Optically anisotropic body (XII-4) | Compound (I-4) of this invention |
| Example 25 | Optically anisotropic body (XII-5) | Compound (I-5) of this invention |
| Example 26 | Optically anisotropic body (XII-6) | Compound (I-6) of this invention |
| Example 27 | Optically anisotropic body (XII-7) | Compound (I-7) of this invention |
| Example 28 | Optically anisotropic body (XII-8) | Compound (I-8) of this invention |
| Example 29 | Optically anisotropic body (XII-9) | Compound (I-9) of ths invention |
| Example 30 | Optically anisotropic body (XII-10) | Compound (I-10) of this invention |
| Comparative | Optically anisotropic body (XII-R-1) | Comparative Compound (R-1) |
| Example 3 | | |
| Comparative | Optically anisotropic body (XII-R-2) | Comparative Compound (R-2) |
| Example 4 | | |

Calculated was a retention rate (defined as retardation retention rate (%)=Re (550) (after test)/Re (550) (before test))×100) of retardation Re (550) before and after a heat resistance/light resistance test of each of the films to be evaluated. The retardation was measured by using an inspection apparatus (RETS-100 manufactured by Otsuka Electronics Co., Ltd.). Also, a degree of discoloration (defined as ΔYI=(YI(after test))−(YI(before test)) before and after the test was determined. Yellowness (YI) was measured by using a spectrophotometer (V-560 manufactured by JASCO Corporation), and yellowness (YI) was calculated by using a color diagnosis program. A calculation formula is the following.

YI=100(1.28X−1.06Z)/Y

(in the formula, YI represents yellowness, and X, Y, and Z represent the tri-stimulus values in a XYZ color system (JIS K7373). The results are shown in a table below.

TABLE 4

| | | Retardation | |
|---|---|---|---|
| Example | Film to be evaluated | retention rate | ΔYI |
| Example 21 | Optically anisotropic body (XII-1) | 95% | 0.4 |
| Example 22 | Optically anisotropic body (XII-2) | 96% | 0.3 |
| Example 23 | Optically anisotropic body (XII-3) | 95% | 0.4 |
| Example 24 | Optically anisotropic body (XII-4) | 89% | 0.7 |
| Example 25 | Optically anisotropic body (XII-5) | 91% | 0.6 |
| Example 26 | Optically anisotropic body (XII-6) | 88% | 0.8 |
| Example 27 | Optically anisotropic body (XII-7) | 90% | 0.7 |
| Example 28 | Optically anisotropic body (XII-8) | 84% | 1.2 |
| Example 29 | Optically anisotropic body (XII-9) | 95% | 0.4 |
| Example 30 | Optically anisotropic body (XII-10) | 89% | 0.7 |
| Comparative | Optically anisotropic body (XII-R-1) | 70% | 5.1 |
| Example 3 | | | |
| Comparative | Optically anisotropic body (XII-R-2) | 65% | 5.9 |
| Example 4 | | | |

The results described above indicate that the compounds represented by the formula (I-1) to the formula (I-10) of the present invention described in Example 1 to Example 10 cause little decrease in retardation and little discoloration. Therefore, the compounds of the present invention are useful as a component of a polymerizable composition. Also, an optically anisotropic body using a polymerizable liquid crystal composition containing each of the compounds of the present invention is useful for application to an optical film and the like.

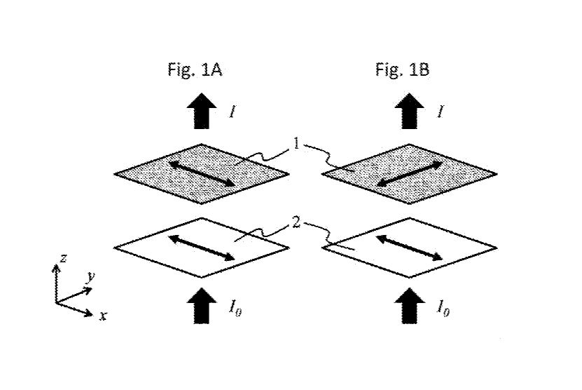

### BRIEF DESCRIPTION OF DRAWINGS

[FIG. 1] an arrangement of a film to be evaluated and a polarization plate, (a): a state in which the alignment direction of a film to be evaluated is parallel to the polarization direction of a polarization plate, (b): a state in which the alignment direction of a film to be evaluated is perpendicular to the polarization direction of a polarization plate, 1: polarization plate (an arrow represents the polarization direction of a polarization plate), 2: a film to be evaluated (an arrow represents the alignment direction), I₀: incident light, I: transmitted light

1. A polymerizable liquid crystal compound, wherein when aligned on a horizontally-aligned substrate, an absorption maximum wavelength λomax in an in-plane direction perpendicular to an alignment direction lies at 320 nm to 420 nm.

2. The compound according to claim 1, wherein when aligned on a horizontally-aligned substrate, at the wavelength λomax, absorbance Ae in the direction parallel to the alignment direction and absorbance Ao in the in-plane direction perpendicular to the alignment direction satisfy a formula (Formula I) below.
Ao/Ae>1 (Formula I)

3. The compound according to claim 1, represented by general formula (I),
P¹—S¹—X¹ₖA¹¹-Z¹¹ₘ₁M¹Z¹²-A¹²ₘ₂R¹ (I)
(in the formula, P¹ represents a polymerizable group; S¹ represents a spacer group or a single bond, and when a plurality of S¹ are present, they may be the same or different; X¹ represents —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH2—, —CH₂—COO—, —CH₂—OCO—, —CH═CH—, —N═N—, —CH═N—N═CH—, —CF═CF—, —C≡C—, or a single bond, and when a plurality of X are present, they may be the same or different (wherein P¹—(S¹—X¹)ₖ— does not contain an —O—O— bond); A¹¹ and A¹² each independently represent a 1,4-phenylene group, a 1,4-cyelohexylene group, a pyridine-2,5-diyl group, a pyrmidine-2,5-diyl group, a naphthalene-2,6-diyl group, a naphthalene-1,4-diyl group, a tetrahydronaphthalene-2,6-diyl group, a decahydronaphthalene-2,6-diyl group, or a 1,3-dioxane-2,5-diyl group, which may be unsubstituted or substituted by one or more L, and when a plurality of A¹¹ and/or A¹² are present, they may be the same or different; Z¹¹ and Z¹² each independently represent —O—, —S—, —OCH₂—, —CH₂O—, —CH₂CH₂—, —CO—, —COO—, —OCO—, —CO—S—, —S—CO—, —O—CO—O—, —CO—NH—, —NH—CO—, —OCO—NH—, —NH—COO—, —NH—CO—NH—, —NH—O—, —O—NH—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —N═N—, —CH═N—, —N═CH—, —CH═N—N═CH—, —CF═CF—, —C≡C—, or a single bond, and when a plurality of Z¹¹ and/or Z¹² are present are present, they may be the same or different; R¹ represents a hydrogen atom, a fluorine atom, a chlorine atom, a bromine atom, an iodine atom, a pentafluorosulfuranyl group, a cyano group, a nitro group, an isocyano group, a thioisocyano group, or a linear or branched alkyl group having 1 to 20 carbon atoms in which one —CH₂— or two or more unadjacent —CH₂— may be each independently substituted by —O—, —S—, —CO—, —COO—, —OCO—, —CO—S—, —S—CO—, —O—CO—O—, —CO—NH—, or —C≡C—, and any hydrogen atom in the alkyl group may be substituted by a fluorine atom, or R¹ may represent a group represented by —(X^{R}—S^{R})_{kR}—P^{R} (wherein P^{R} represents a polymerizable group; S^{R} represents a spacer group or a single bond, and when a plurality of S^{R} are present, they may be the same or different; X^{R} represents —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —N═N—, —CH═N—N═CH—, —CF═CF—, —C≡C—, or a single bond, and when a plurality of X^{R} are present, they may be the same or different (wherein —(X^{R}—S^{R})_{kR}—P^{R} does not contain an —O—O— bond): and kR represents an integer of 0 to 8); M¹ represents a divalent hydrocarbon group containing a conjugated system; L represents a fluorine atom, a chlorine atom, a bromine atom, an iodine atom, a pentafluorosulfuranyl group, a nitro group, a cyano group, an isocyano group, an amino group, a hydroxyl group, a mereapto group, a methylamino group, a dimethylamino group, a diethylamino group, a diisopropylamino group, a trimetliylsilyl group, a dimethylsilyl group, a thioisocyano group, or a linear or branched alkyl group having 1 to 20 carbon atoms in which one —CH₂— or two or more unadjacent —CH₂— may be each independently substituted by —O—, —S—, —CO—, —COO—, —OCO—, —CO—S—, —S—CO—, —O—CO—O—, —CO—NH—, —NH—CO—, —CH═CH—COO—, —CH═CH—OCO—, —COO—CH═CH—, —OCO—CH═CH—, —CH═CH—, —CF═CF—, or —C≡C—, and when a plurality of L are present, they may be the same or different, and any hydrogen atom in the alkyl group may be substituted by a fluorine atom, or L may represent a group represented by —(X^{L}—S^{L})_{kL}—P^{L} (wherein P^{L} represents a polymerizable group; S^{L} represents a spacer group or a single bond, and when a plurality of S are present, they may be the same or different; X^{L} represents —O—, —S—, —OCH₂—, —CH₂O—, —CO—, —COO—, —OCO—, —CO—S—, —S—CO—, —O—CO—O—, —CO—NH—, —NH—CO—, —SCH₂—, —CH₂S—, —CF₂O—, —OCF₂—, —CF₂S—, —SCF₂—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH₂—, —CH₂—COO—, —CH₂—OCO—, —CH═CH—, —N═N—, —CH═N—N═CH—, —CF═CF—, —C≡C—, or a single bond, and when a plurality of X are present, they may be the same or different (wherein —(X^{L}—S^{L})_{kL}—P^{L} does not contain an —O—O— bond); and kL represents an integer of 0 to 8); k represents an integer of 0 to 8; m1 and m2 each independently represent an integer of 0 to 5, and m1+m2 represents an integer of 1 to 5).

4. The compound according to claim 3, wherein in the general formula (I), P¹ represents a group selected from formula (P-1) to formula (P-20) below.

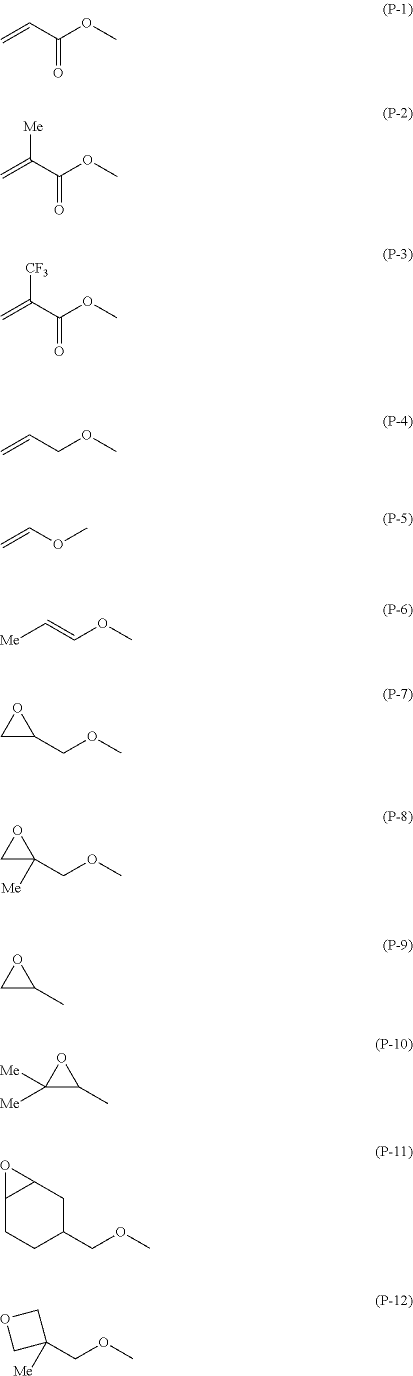

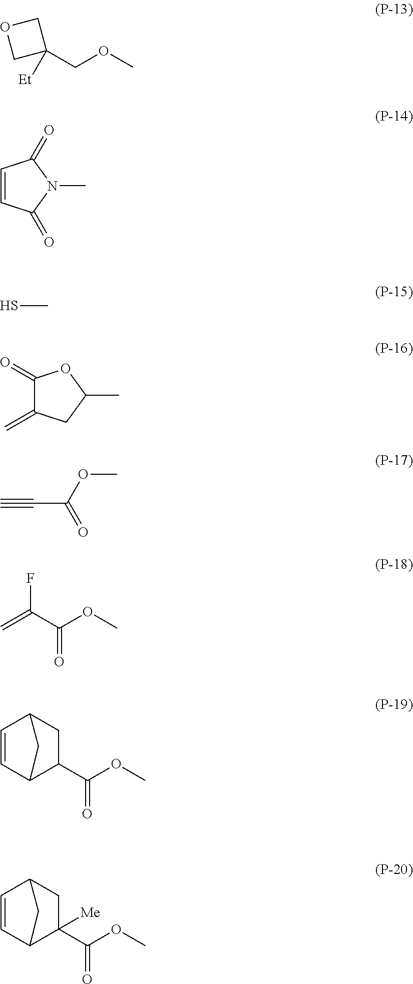

5. The compound according to claim 3, wherein in the general formula (I), S¹ each independently represent an alkylene group having 1 to 20 carbon atoms in which one —CH₂— or two or more unadjacent —CH₂— may be each independently substituted by —O—, —COO—, —OCO—, —OCO—O—, —CO—NH—, —NH—CO—, —CH═CH—, or —C≡C—.

6. The compound according to claim 3, wherein in the general formula (I), the total number of π electrons contained in M¹ is 4 to 50.

7. A composition comprising the compound according to claim 1.

8. A liquid crystal composition comprising the compound according to claim 1.

9. A polymer produced by polymerizing the composition according to claim 7.

10. An optically anisotropic body comprising the polymer according to claim 9.

11. The optically anisotropic body according to claim 10, wherein when aligned on a horizontally-aligned substrate, an absorption maximum wavelength λomax in an in-plane direction perpendicular to an alignment direction lies at 320 nm to 420 nm.

12. The optically anisotropic body according to claim 10, wherein when aligned on a horizontally-aligned substrate, at the wavelength λomax, absorbance Ae in the direction parallel to the alignment direction and absorbance Ao in the in-plane direction perpendicular to the alignment direction satisfy a formula (Formula I) below.
Ao/Ae>1 (Formula I)

13. A resin, a resin additive, an oil, a filter, an adhesive, a pressure-sensitive adhesive, oil and fat, an ink, a medical product, a cosmetic, a detergent, a building material, a packaging material, a liquid crystal material, an organic EL material, an organic semiconductor material, an electronic material, a display element, an electronic device, a communication device, an automobile component, an aircraft component, a machine component, an agricultural chemical or food, or a product using the same, which uses the compound according to claim 1.
